(19) United States
(12) Patent Application Publication
(10) Pub. No.: US 2006/0105445 A1
(43) Pub. Date: May 18, 2006

(54) Medium and method for enriching, purifying or depleting atp binding proteins from a pool of proteins

(76) Inventors: Klaus Godl, Durer-Str.18A (DE); Andrea Missio, Munich (DE); Henrik Daub, Munich (DE); Matthias Stein-Gerlach, Munich (DE); Zoltan Greff, Budapest (HU); Gyorgy Keri, Budapest (HU); Laszlo Orfi, Budapest (HU); Zoltan Varga, Kolesd (HU); Bert Klebl, Gunzelhofen (DE)

Correspondence Address: J.C. Patents, Suite 250, 4 Venture,, Irvine, CA, 92618, US

(21) Appl. No.: 10/523,577

(22) PCT Filed: Jul. 29, 2003
(86) PCT No.: PCT/EP03/08375

§ 371 Date: Aug. 22, 2005

(51) Int. Cl. C12N 9/12; C07K 14/705

(52) U.S. Cl. 435/194; 530/350

(57) ABSTRACT

The present invention relates to a medium and a method for enriching ATP binding proteins, e.g. proteinkinases, from a pool of proteins, like a proteome. The medium of the present invention comprises specific inhibitors, e.g. at least one of the compounds 4-[4-(4-fluoro-phenyl)-5-pyridine-4-yl-1H-imidazole-2-yl]-benzylamine, 2-[4-(2-Amino-ethoxy)-phenylamino]-6-(2,6-dichloro-phenyl)-8-methyl-8H-pyrido[2,3-d]pyrimidine-7-one, 2-[1-(3-aminopropyl)-1H-indole-3-yl)maleinmide, 3-[1-(3-Aminopropyl)-1H-indol-3-yl]-3-(1H-indol 3-yl)-maleinmide, 3-[1-(3-Aminopropyl)-1H-indol-3-yl]4-(1-methyl-1H-indol-3-yl) maleinimide, 3-(8-Aminomethyl-6,7,8,9-tetrahydropyrido-[1,2-a]-indol-10-yl) (1-methyl-1H-indol -3-yl)-maleinmide, [6-(3-Amino-propoxy)-methoxy-quinazolin-4-yl]-(3-chloro-phenyl)-amine, 6-(3-Amino-propoxy)-7-methoxy-quinazolin-4-yl-(3-chloro-4-fluoro-phenyl)-amine, 6-(3Amino-propoxy)-7-methoxy-quinazolin-4-yl]-(3-bromo-phenyl)-amine and 4-[4-(4-Amino-butyl)-piperazin-1-yl-methyl]-N-[4-methyl-3-(4-pyridin-3-yl-pyrimidin-2-ylamino)-phenyl]-benzamide immobilized on a support material. According to the method of the present invention the above-mentioned immobilized compounds are used to selectively bind protein kinases from a pool of heterogeneous proteins.

The present invention refers to a medium and a method for enriching, purifying or depleting ATP binding proteins from a pool of proteins, such as a proteome.

ATP binding proteins play an important role in the metabolism of an organism. E.g., enzymes of the protein kinase family are essential switches of the cellular signal transduction machinery in all eucaryotic cells. They have been implicated with the control of numerous physiological and pathophysiological processes in eucaryotic organisms and therefore represent an important class of drug targets for a variety of indications such as cancer, inflammation and infectious diseases. Biochemical identification of protein kinases relevant for disease progression has been a rather difficult methodological challenge in the past and there is a clear need for novel and innovative techniques which allow rapid and systematic biochemical analysis of all cellular kinase activities. The most efficient established techniques for parallel analysis of cellular proteins are two-dimensional gel electrophoresis in combination with mass spectrometry for identification of separated protein spots. But due to the enormous complexity of the proteome of an individual, this approach has not been successful for identification of protein kinase targets, since most protein kinases are low abundance proteins that are not detectable if unfractionated cellular extracts are used for proteome analysis. Thus, efficient and selective enrichment is a prerequisite for subsequent identification of protein kinase targets by a proteomics approach. As no efficient pre-fractionation techniques have been reported to date, novel experimental approaches are required to accomplish these tasks.

It is therefore the object of the present invention to provide a medium and a method which are capable of enriching, purifying or depleting ATP binding proteins from a pool of proteins, such as a proteome, a cell lysate or a tissue lysate.

This object is solved by the medium according to independent claim 1 and the method according to independent claim 12. Further advantageous features, aspects, and details of the invention are evident from the dependent claims, the description, the examples, and the drawings.

According to one aspect, the present invention relates to a medium for separating at least one ATP binding protein from a pool of proteins, like a proteome of an individual, the medium comprising at least one compound of the general formula I 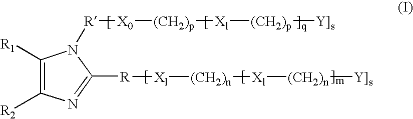
formulas IIa and IIb (compound class B) 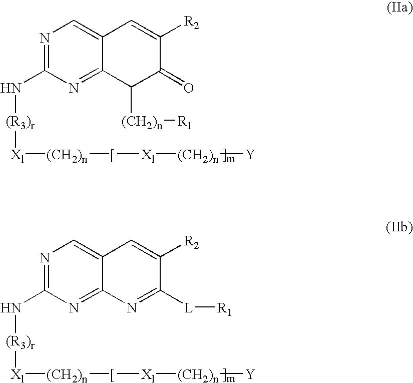
wherein
each L is independently selected from —NH—CO—NH—, —NH—SO₂—, or —NH—CS—NH,
each X is independently selected from —CH₂—, —NH—, —O—, —S—, 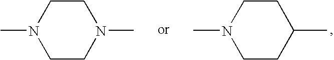
each Y is independently selected from —NH₂, —NHR₁, —OH, —SH or —SO(CH₃),
each l is independently selected to be 0 or 1,
each m is independently selected to be an integer from 0 to 10,
each n is independently selected to be an integer from 0 to 10,
each o is independently selected to be 0 or 1,
each p is independently selected to be an integer from 0 to 10,
each q is independently selected to be an integer from 0 to 10,
each r is independently selected to be an integer from 0 to 2,
R and R′ are independently of each other —H, 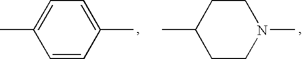
and each s is independently selected to be 0 or 1, with the provisio that s=0 if R or R′ is H,
each R₁ is independently selected from —H, C₁-C₆ alkyl (linear or branched), bicyclo[3.3.1]heptanyl,or an unsubstituted or partially or fully substituted C₃-C₈ cycloalkyl, aryl, pyridinyl or pyrimidinyl, substituted by —F, —Cl, —Br, —I, —CN, —OH, —SH, —NH₂, —NHCHR₂R₂, C₁-C₆-alkoxy, C₁-C₆-alkylthio, C₁-C₆-haloalkyloxy, partially or fully halogenated C₁-C₆ alkyl and/or —X₁—(CH₂)ₙ—Y (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
each R₂ is independently selected from —H, C₁-C₆ alkyl (linear or branched), an unsubstituted or partially or fully substituted aryl,substituted by —F, Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), and
each R₃ is independently selected from X, an unsubstituted or partially or fully substituted aryl, pyridinyl or pyrimidinyl, substituted by —F, —Cl, —Br, —I, —CN, —OH, —SH, —NH₂, —NHCHR₁R₁, C₁-C₆-alkoxy, C₁-C₆-alkylthio, C₁-C₆-haloalkyloxy, and/or partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
formulas IIIa and IIIb (compound class C) 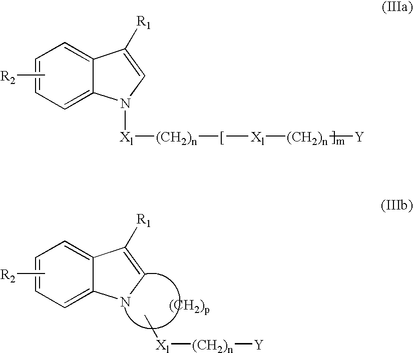
wherein
each R₁ is independently selected from 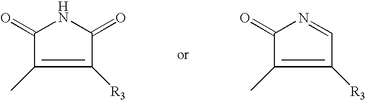
each R₃ is independently selected from -indolyl, N—(C₁-C₆ alkyl) -indolyl (alkyl is linear or branched), —NHR₁′,
—S—R₁′, or —O—R₁′,
R₁′ is —H, C₁-C₆ alkyl (linear or branched) or aryl,
each R₂ is independently selected from —H, —F, —Cl, —Br, —I, —CN, —OH, —SH, —NH₂, C₁-C₆-alkyl (linear or branched), C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
each X is independently selected from —CH₂—, —NH—, —O—, —S—, 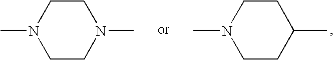
each Y is independently selected from —NH₂, —NHR₁, —OH, —SH or —SO(CH₃), each 1 is independently selected to be 0 or 1,
m is an integer from 0 to 10,
each n is independently selected to be an integer from 0 to 10,
p is an integer from 2 to 6,
formula IV (compound class D) 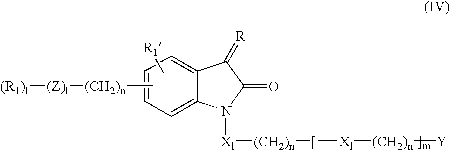
wherein
each X is independently selected from —CH₂—, —NH—, —O—, —S—, 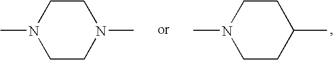
each Y is independently selected from —NH₂, —NHR₁, —OH, —SH or —SO(CH₃), Z is —SO₂—NR₁R, —CO, —O—CO—, —NH—CO, —COO—, —CO—NH, —OCH₂—, —SCH₂—,
each 1 is independently selected to be 0 or 1,
m is an integer from 0 to 10,
each n is independently selected to be an integer from 0 to 10,
R is —CR₁L, —N—NH-L
each R₁ is independently selected from —H, C₁-C₆ alkyl (linear or branched), unsubstituted or partially or fully substituted aryl, pyridinyl, pyriridinyl, C₃-C₈ cycloalkyl substituted by —F, —Cl, —Br, —I, —CN, —OH, —SH, NH₂, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group, C₁-C₆-alkylthio denotes an S-alkyl group, C₁-C₆-haloalkyloxy denotes an halogen-alkyl-O group, C₁-C₆-haloalkyl denotes an halogen-alkyl group wherein the alkyl group is linear or branched), —F, —Cl, —Br, —I, —COOH, —CH₂)ₙ—OH, oxazolyl, thiazolyl, thiophenyl, pyrrolyl, furanyl, imidazolyl, pyrazolyl, pyridinyl, pyrimidinyl, R₁′ is independently selected from H or R₁ and R₁′ may form together the following substituted ringsystem 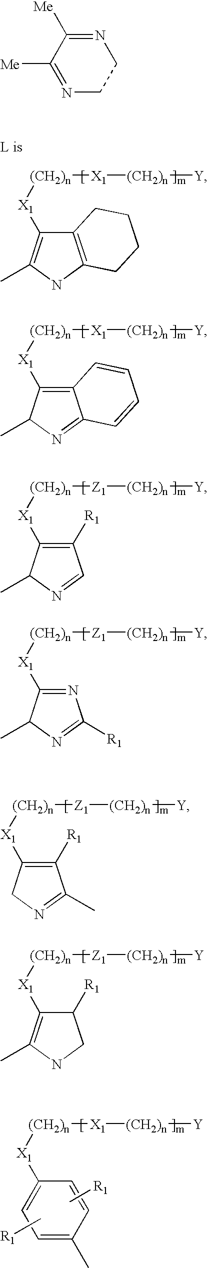
formulas V and VI (compound class E and F) 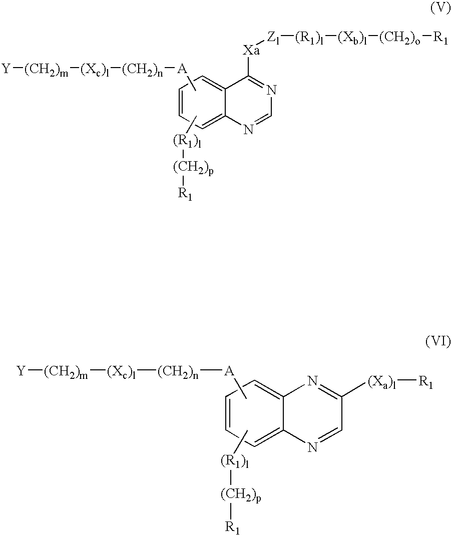
wherein
A, Xₐ, X_{b}, and X_{c}, are independently selected to be Z, —CH—, —NH—, —O—, —S—, 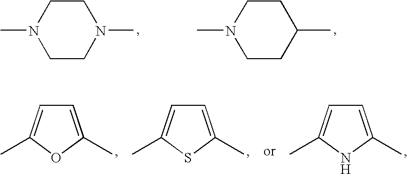
each Y is independently —NH₂, —NHR₁, —OH, —SH or —SO(CH₃),
each Z is independently selected from —SO₂—NR₁—, —CO, —O—CO—, —NH—CO—, —COO—, —CO—NH—, —CS—NH—, —OCH₂—, —SCH₂—, or —NH—CO—NH—,
each 1 is independently selected to be 0 or 1,
each m is independently selected to be an integer from 0 to 10,
each n is independently selected to be an integer from 0 to 10,
each o is independently selected to be an integer from 0 to 10,
each p is independently selected to be an integer from 0 to 10,
each R₁ is independently selected from —H, —O—, C₁-C₆ alkyl linear or branched), C₁-C₆-alkoxy, C₁-C₆-alkylthio, C₁-C₆-haloalkyloxy, C₁-C₆ partially or fully halogenated alkyl, unsubstituted or partially or fully substituted C₃-C₈ cycloalkyl, an unsubstituted or partially or fully substituted aryl, wherein the cycloalkyl and the aryl are optionally substituted by —F, —Cl, —Br, —I, —CN, —OH, —SH , —NH₂, —CONH₂, C₁-C₆ allyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), 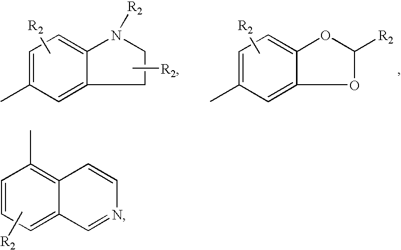
each R₂ is independently selected from —F, —Cl, —Br, —I, —CN, —OH, —SH, NH₂, C₁-C₆ alkyl ear or branched), C₁-C₆-alkoxy, C₁-C₆-alkylthio, C₁-C₆-haloalkyloxy, partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
formula VII (compound class G) 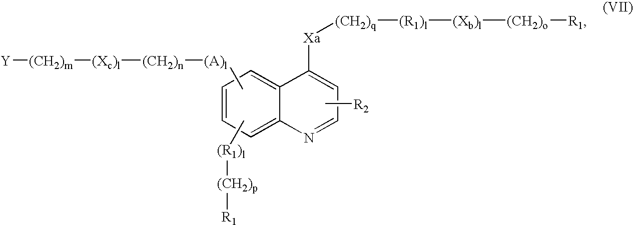
formula VIII (compound class H) 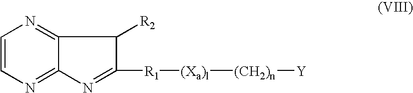
, formula IX (compound class I) 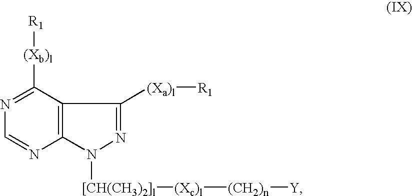
formula X (compound class J) 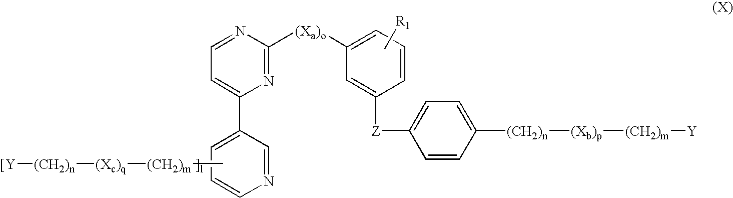
or formula XI (compound class K) 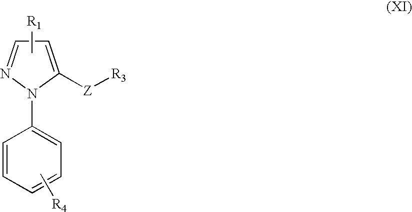
wherein
A, Xₐ, X_{b}, and X_{c} are independently selected from Z, —CH₂—, —NH—, —O—, —S—, 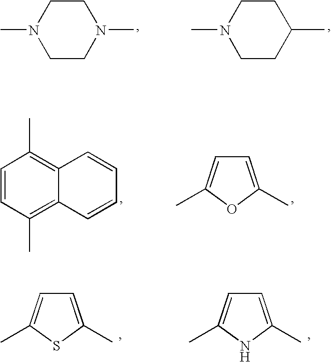
each Y is independently selected from —H, —NH₂, —NHR₁, —OH, —SH or —SO(CH₃),
each Z is independently selected from —SO₂—NR₁—, —CO, —O—CO—, —NH—CO—, —COO—, —CO—NH—, —OCH₂—, —SCH₂—, —NH—CO—NH—,
each 1 is independently selected to be 0 or 1,
each m is independently selected to be an integer from 0 to 10,
each n is an integer independently selected from 0 to 10,
each of o, p, q is an integer independently selected from 0 to 10,
each R₁ is independently selected from —H, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), —F, —Cl, —Br, —I, —COOH, —NH₂, 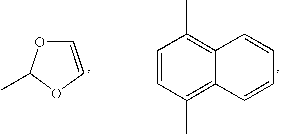
each R₂ is independently selected from —F, —Cl, —Br, —I, —CN, —OH, —SH, NH₂, C₁-C₆ alkyl (linear or branched), C₁-C₆-alkoxy, C₁-C₆-alkylthio, C₁-C₆-haloalkyloxy, partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
R₃ is —H or (R₁)₁—(Xₐ)₁—(CH₂)ₙ—(X_{b})₁—(CH₂)ₙ(Y)₁—R₁,
R₄ is —H or —(R₁)₁—(Z)₁—(CH₂)ₙ—(X_{b})₁—(CH₂)ₙ—(Y)₁—R₁

immobilized on a support material.

It is preferred that the compounds of the compound classes A to K according to the general formulas I to XI are covalently bound to the support material. It is clear that to achieve such a covalent bond one radical, preferably a hydrogen radical must be removed from the respective compound to form such a bond with the support material. It is furthermore preferred that these compounds are bonded to the support material via a group Y.

Furthermore, it is to be understood that in those cases in which the substituents R₁ and R₃ listed for the compounds according to formulas IIa, IIb, V, VI, and VII are bonded to two of the other possible substituents or groups instead of only one, these groups R₁ and R₃ are to be understood as the corresponding diradical groups, e.g. the C₁₋₆ alkyl radical group as a C₁₋₆ alkandiyl-radical group (or C₁₋₆ alkylene group).

In a preferred embodiment, the index r in compounds according to formula IIa and IIb is selected to be 0. In a further preferred embodiment, in the groups —(R₁)₁—(CH₂)ₚ— or Z₁—(R₁)₁—(X_{b})₁— in the compounds according to formulas (V) and (VI) l and p are each selected to be 0.

As used in the definitions of the formulas I to XI above, C₁-C₆ alkyl represents —CH₃, —C₂H₅, —C₃H₇, —CH(CH₃)₂, —C₄H₉, —C(CH₃)₃, —CH(CH₃)—CH₂—CH₃, —CH₂—CH(CH₃) —CH₃, —C₅H₁₁, —(CH₂)₂—CH(CH₃)₂, —CH(CH₃)—(CH₂)₂—CH₃, —CH₂—CH(CH₃)—C₂H₅, —C₆H₁₃, —CH(CH₃)—(CH₂)₃—CH₃, —(CH₂)₃—CH(CH₃)₂, —(CH₂)₃—CH(CH₃)—CH₃, —(CH₂)₂—CH(CH₃)—CH₂—CH₃, or —CH₂—CH(CH₃)—(CH₂)₃—CH₃.

As used in the definitions of formulas I to XI above C₃-C₈ cycloalkyl represents compounds having the following structures:

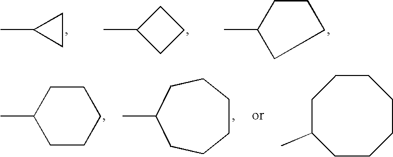

Particularly preferred are from the compound class A 4-[4-(4Fluoro-phenyl)-5-pyridine-4-yl-1H-imidazole-2-yl]-benzylamine (“compound A”), from the compound class B 2-[4-(2-Amino-ethoxy)-phenylamino]-6-(2,6-dichloro-phenyl)-8-methyl-8H-pyrido[2,3-d]pyrimidine-7-one (“compound B”), from the compound class C 3-[1-(3-Aminopropyl)-1H-indole-3-yl]-3-(1H-indole-3-yl)-maleinimide (“compound C”), 3-[1-(3-Aminopropyl)-1H-indole-3-yl]-4-(1-methyl-1H-indole-3-yl)maleinimide (“compound D”) and 3-(8-Aminomethyl-6,7,8,9-tetrahydro-pyrido[1,2-a]-indole-10-yl)-4-(1-methyl-1H-indole-3-yl)-maleinimide (“compound E”), from the compound class E [6-(3-Amino-propoxy)-7-methoxy-quinazoline-4-yl]-(3-chloro-phenyl)-amine (“compound F”), 6-(3-Amino-propoxy)-7-methoxy-quinazoline-4-yl]-(3-chloro-4-fluoro-phenyl)-amine (“compound G”) and 6-(3-Amino-propoxy)-7-methoxy-quinazoline-4-yl]-(3-bromo-phenyl)-amine (“compound H”) and from the compound class J 4-[4-(4-Amino-butyl)-piperazine-1-yl-methyl]-N-[4-methyl-3-(4-pyridine-3-yl-pyrimidine-2-ylamino)-phenyl]-benzamide (“compound I”) immobilized on a support material.

According to a further preferred aspect, the support material comprises or consists of an agarose material, particularly a modified agarose-material like an epoxy-activated Sepharose 6B material (Sepharose is obtainable from Amersham Biosciences). It is especially preferred if the support material for the compound classes A to K is the modified agarose material referred to above.

In a further preferred aspect of this invention, at least on of each of the compounds according to the generic formulas I to XI is immobilized on the support material. In other aspects of the invention a subselection of compounds I to XI is immobilized on the substrate material. This selection can be made according to the specific nature of the ATP binding proteins which are to be enriched, purified or depleted from the pool of proteins used.

In a further preferred embodiment of the present invention, the medium comprises at least one of the above listed compounds IIIb to XI, i.e. IIIb, IV, V, VI, VII, VIII, IX, X and/or to XI immobilized on a support material. In a further preferred embodiment the medium comprises at least one of the above listed compounds IV to XI, i.e. IV, V, VI, VII, VIII, IX, X and/or to XI immobilized on a support material.

According to a still further aspect, the support material comprises or consists of ferro- or ferrimagnetic particles as e.g. known from WO 01171732, incorporated herein by reference as far as properties of ferro- or ferrimagnetic particles are concerned. The ferro- or ferrimagnetic particles may comprise glass or plastic. The ferro- or ferrimagnetic particles that can be used with the present invention may be porous. The ferro- or ferrimagnetic glass particles may comprise about 30 to 50% by weight of Fe₃O₄ and about 50 to 70% by weight of SiO₂. The ferro- or fernmagnetic particles used herein preferably have an average size of about 5 to 25 μm in diameter, more preferably about 6 to 15 μm, and particularly about 7 to 10 μm. The total surface area of the ferro- or ferrimagnetic particles may be 190 g/m² or greater, e.g. in the range of about 190 to 270 g/m² (as determined according the Brunaur Emmet Teller (BET) method).

These magnetic particles facilitate purification, separation and/or assay of biomolecules, like protein kinases. Magnetic particles (or beads) that bind a molecule of interest can be collected or retrieved by applying an external magnetic field to a container comprising the particles. Unbound molecules and supernatant liquid can be separated from the particles or discarded, and the molecules bound to the magnetic particles may be eluted in an enriched state.

Although in the following it is described that compounds of classes A to K (formulas I to XI) were used separately bound to the support material, it is clear that also any combination of the immobilized compounds can be used according to the present invention to enrich, purify or deplete ATP binding proteins from a pool of different proteins, like from a proteome.

According to another aspect, the present invention refers to a method for enriching, purifying or depleting at least one ATP binding protein, e.g. a protein kinase, from a pool of proteins containing at least one such ATP binding protein, the method comprising the following steps (a) immobilizing at least one of the compounds of the compound classes A to K (compounds of the formulas I to XI) as described above on a support material, (b) bringing the pool of proteins containing at least one ATP binding protein into contact with at least one of the immobilized compounds of the compound classes A to K (compounds of the formulas I to XI), and (c) separating the proteins not bound to the at least one compound of the compound classes A to K (compounds of the formulas I to XI) immobilized on the support material from the at least one ATP binding protein bound to the compound of the compound classes A to K (compounds of the formulas I to XI) immobilized on the support material.

According to still further preferred aspect, in step (a) at least one of the compounds 4-[4-(4-fluoro-phenyl)-5-pyridine-4-yl-1H-imidazole-2-yl]-benzylamine, 2-[4-(2-amino-ethoxy)-phenylamino]-6-(2,6-dichloro-phenyl)-8-methyl-8H-pyrido[2,3-d]pyrimidine-7-one, 3-[1-(3-aminopropyl)-1H-indole-3-yl]-3-(1H-indole-3-yl)-maleinimide, 3-[1-(3-aminopropyl)-1H-indole-3-yl]-4-(1-methyl-1H-indole-3-yl)maleinimide, 3-(8-aminomethyl-6,7,8,9-tetrahydropyrido[1,2-a]-indole-10-yl)-4-(1-methyl-1H-indole-3-yl)-maleinimide, [6-(3-amino-propoxy)-7-methoxy-quinazoline-4-yl]-(3-chloro-phenyl)-amine, 6-(3-amino-propoxy)-7-methoxy-quinazoline-4-yl]-(3-chloro-4-fluoro-phenyl)-amine, 6-(3-amino-propoxy)-7-methoxy-quinazoline-4-yl]-(3-bromo-phenyl)-amine, and 4-[4(4-amino-butyl)-piperazin-1-yl-methyl]-N-[4-methyl-3-(4-pyridine-3-yl-pyrimidine-2-ylamino)-phenyl]-benzamide is immobilized on the support material; in step (b) the pool of proteins containing at least one ATP binding protein is brought into contact with at least one of the compounds 4-[4-(4-fluoro-phenyl)-5-pyridine-4-yl-1H-imidazole-2-yl]-benzylamine, 2-[4-(amino-ethoxy)-phenylamino]-6-2,6-dichloro-phenyl)-8-methyl-8H-pyrido[2,3-d]pyrimidine-7-one, 3-[1-(3-aminopropyl)-1H-indole-3-yl]-3-(1H-indole-3-yl)-maleinimide, 3-[1-(3-aminopropyl)-1H-indole-3-yl]-4-(1-methyl-1H-indole-3-yl)maleinimide, 3-(8-aminomethyl-6,7,8,9-tetrahydro-pyrido[1,2-a]-indole-10-yl)-4-(1-methyl-1H-indole-3-yl)-maleinimide, [6-(3-amino-propoxy)-7-methoxy-quinazoline-4-yl]-(3-chloro-phenyl)-amine, 6-(3-amino-propoxy)-7-methoxy-quinazoline-4-yl]-(3-chloro-4-fluoro-phenyl)-amine, 6-(3-amino-propoxy)-7-methoxy-quinazoline4-yl]-(3-bromo-phenyl)-amine and 4-[4-(4-amino-butyl)-piperazine-1-yl-methyl]-N-[4-methyl-3-(4-pyridine-3-yl-pyrimidine-2-ylamino)-phenyl]-benzamide immobilized on the support material; and in step (c) the proteins not bound to the at least one compound 4-[4-4-fluoro-phenyl)-5-pyridine-4-yl-1H-imidazole-2-yl]-benzylamine, 2-[4-(2-amino-ethoxy)-phenylamino]-6-(2,6-dichloro-phenyl)-8-methyl-8H-pyrido[2,3-d]pyrimidine-7-one, 3-[1-(3-aminopropyl)-1H-indole-3-yl]-3-(1H-indole-3-yl)-maleinimide, 3-[1-(3-aminopropyl)-1H-indole-3-yl]-4-(1-methyl-1H-indole-3-yl) maleinimide, 3-(8-aminomethyl-6,7,8,9-tetrahydro-pyrido[1,2-a]-indole-10-yl)-4-(1-methyl-1H-indole-3-yl)-maleinimide, [6-(3-amino-propoxy)-7-methoxy-quinazoline-4-yl]-(3-chloro-phenyl)-amine, 6-(3-amino-propoxy)-7-methoxy-quinazoline-4-yl]-(3-chloro-4-fluoro-phenyl)-amine, 6-(3-amino-propoxy)-7-methoxy-quinazoline-4-yl]-(3-bromo-phenyl)-amine, and 4-[4-(4-amino-butyl)-piperazine-1-yl-methyl]-N-[4-methyl-3-(4-pyridine-3-yl-pyrimidine-2-ylamino)-phenyl]-benzamide immobilized on the support material are separated from the at least one ATP binding protein bound to the compound 4-[4-4-fluoro-phenyl)-5-pyridine-4-yl-1H-imidazole-2-yl]-benzylamine, 2-[4-(2-amino-ethoxy)phenylamino]-6-(2,6-chloro-phenyl)-8-methyl-8H-pyrido[2,3-d]pyrimidine-7-one, 3-[1-(3-aminopropyl)-1H-indole-3-yl]-3-(1H-indole-3-yl)-maleinimide, 3-[1-(3-aminopropyl)-1H-indole-3-yl]-4-(1-methyl-1H-indole-3-yl) maleinimide, 3-(8-aminomethyl-6,7,8,9-tetrahydro-pyrido[1,2a]-indole-10-yl)-4-(1-methyl-1H-indole-3-yl)-maleinimide, [6-(3-amino-propoxy)-7-methoxy-quinazoline-4-yl]-(3-chlorophenyl)-amine, 6-(3-amino-propoxy)-7-methoxy-quinazoline-4-yl]-(3-chloro-4-fluoro-phenyl)-amine, 6-(3-amino-propoxy)-7-methoxy-quinazoline-4-yl]-(3-bromo-phenyl)-amine, and 4-[4-(4-amino-butyl)-piperazine-1-yl-methyl]-N-[4-methyl-3-(4pyridine-3-yl-pyrimidine-2-ylamino)-phenyl]-benzamide immobilized on the support material.

According to a still further preferred embodiment, the method of the present invention comprises a further step (d) releasing the at least one ATP binding protein bound to the at least one compound of the compound classes A to K (formulas I to XI) immobilized on the support material from the at least one of said compounds. This releasing is preferably effected with a buffer containing the respective immobilized compound plus ATP (in this context it is clear that the “immobilized compound” contained in the releasing buffer is not the one fixed to the support material, but of course another amount of the same material).

According to a still further aspect, the method according to the present invention comprises further a step (e) collecting the at least one ATP binding protein released from the immobilized compound(s) of the compound classes A to K.

There were identified eleven structurally unrelated compound classes with primary amine substituents assumed to refer to the ATP binding sites of ATP binding proteins, like protein kinases, which have ideal properties for immobilization on solid support materials via the primary amines. These compound classes are the classes A to K represented by the general formulas I to XI described in detail above. Among those compounds falling under the general formulas I to XI, compound A, i.e. 4-[4-(4-fluoro-phenyl)-5-pyridine-4-yl-1H-imidazol-2-yl]-benzylamine; compound B, i.e. 2-[4-(2-amino-ethoxy)-phenylamino]-6-(2,6-dichlorophenyl)-8-methyl-8H-pyrido[2,3-d]pyrimidine-7-one, compound C, D and B, i.e. 2-[1-(3-aminopropyl)-1H-indole-3-yl]-3-(1H-indole-3-yl) maleinimide, 3-[1-(3-aminopropyl)-1H-indole-3-yl]-4-(1-methyl-1H-indole-3-yl) maleinimide, and 3-8-aminomethyl-6,7,8,9-tetrahydro-pyrido[1,2-a]-indole-10-yl)-4-(1-methyl-1H-indole-3-yl)-maleinimide, compound F, G and H, i.e. [6-(3-amino-propoxy)-7-methoxy-quinazoline-4-yl]-(3-chloro-phenyl)-amine, 6-(3-amino-propoxy)-7-methoxy-quinazoline-4-yl]-(3-chloro-4-fluoro-phenyl)-amine, and 6-(3-amino-propoxy)-7-methoxy-quinazoline-4-yl]-(3-bromo-phenyl)-amine and compound I, i.e. from the compound class J 4-[(4-amino-butyl)-piperazine-1-yl-methyl]-N-[4-methyl-3-(4-pyridine-3-yl-pyrimidine-2-ylamino)-phenyl]-benzamide are particularly preferred.

Each of the compounds falling under the general formulas I to XI can be coupled to a support material, e.g. a modified agarose material (e.g. epoxy-activated Sepharose 6B modified by reaction of the compounds' primary amines with the epoxy group of the 1,4-bis(2,3-epoxypropoxy)-butane spacer of the epoxy-activated Sepharose 6B beads) or the ferro- or ferrimagnetic particles described in more detail above. The coupling of the compounds of the compound classes A to K to the support material according to a preferred embodiment of the invention is covalently. The novel reagents (compounds A to I plus solid support material) are referred to in the following as Kinator I (containing immobilized compound A), Kinator II (containing immobilized compound B) and Kinator III (containing immobilized compound C), Kinator IV (containing immobilized compound D), Kinator V (containing immobilized compound E). Kinator VI (containing immobilized compound F), Kinator VII (containing immobilized compound G), Kinator VIII (containing immobilized compound H) Kinator IX (containing immobilized compound I). Epoxy-activated Sepharose 6B was chosen as a preferred support material since it provides a long hydrophilic 12 atom spacer, thereby minimizing the risk of a sterical clash of a protein kinase bound to the immobilized inhibitor with the resin polymer of the support material.

Based on a variety of newly defined criteria for a novel selection scheme to identify suitable compounds for novel kinase target identification applications, there were identified eleven compound classes suitable for covalent coupling to a solid support material. By reacting the compounds A-I falling under the general formulas I to III, V and X with epoxy-activated Sepharose 6B, nine novel reagents named Kinator I to IX were generated that had not been reported before. The novel reagents have the ability to selectively bind sets of endogenously expressed cellular ATP binding proteins, like protein kinases, thereby efficiently enriching, purifying or depleting ATP binding proteins from total cell extracts. Furthermore, a novel elution procedure for affinity chromatography was developed on matrices containing bound ATP binding protein inhibitors, like protein kinase inhibitors, which depends on concomitant addition of both free compound (inhibitor) and ATP for quantitative ATP binding protein elution from the Kinator chromatography media and related chromatography media. Thus, the present invention relates to the conception and generation of these novel separation matrices and their application for the purpose of affinity purification of ATP binding proteins like protein kinases.

Due to the enormous complexity of the proteome, approaches to identify ATP binding protein targets have not been successful previously, since most of the ATP binding proteins are low abundance proteins that are not detectable if unfractionated cellular extracts are used for proteome analysis. Thus, efficient and selective enrichment and/or purification is a prerequisite for subsequent identification of ATP binding protein targets, like protein kinase targets, by a proteomics approach. With the present invention, it is possible for the first time to selectively enrich or purify ATP binding proteins like protein kinases from a heterogeneous pool of proteins. As will be shown below, the concentration of ATP binding proteins can be increased by e.g. a hundred times using the medium and method according to the present invention. It was shown according to the present invention that the inventive media efficiently bind subsets of endogenously expressed ATP binding proteins by in vitro interaction studies. Furthermore, a novel elution protocol could be established which allowed specific elution of a representative ATP binding protein, such as a specific protein kinase, from the inventive media under non-denaturing conditions.

The buffer used to separate the bound ATP binding proteins from the proteins not bound preferably contains from 5 to 500 mM Hepes/NaOH pH 6.5 to 8.5 and/or 5 to 500 mM Tris-HCl pH 6.8 to 9.0, 0 to 1000 mM NaCl, 0 to 5% Triton X-100, 0 to 500 mM EDTA, and 0 to 200 mM EGTA. If the buffer is used to release the bound ATP binding protein(s), it contains furthermore preferably 1 to 100 mM ATP, 1-200 mM MgCl₂ and 0.1 to 10 mM of at least one of the compounds of the compound classes A to K, particularly e.g. 4-[4-(4-fluoro-phenyl)-5-pyridine-4-yl-1H-imidazole-2-yl]-benzylamine, 2-[4-(amino-ethoxy)-phenylamino]-6-(2,6-dichloro-phenyl)-8-methyl-8H-pyrido (2,3-d]pyrimidine-7-one, and 2-[1-(3-aminopropyl)-1H-indole-3-yl) maleinimide.

Specifically, the ATP binding proteins, e.g. protein kinases, could be enriched from the pool of proteins used as the starting material with the medium or the method according to the present invention at least 100-fold, e.g. 100- to 1000-fold.

According to a particularly preferred embodiment of the present invention, the pool of proteins from which the at least one ATP binding protein is separated contains a high salt concentration. “Hgh salt concentration” means according to the present invention a concentration of 0.5 to 5 M, preferably 0.5 to 3 M, more preferably from 0.75 to 2 M and particularly about 1 M. Every salt may be used which does not occupy the ATP binding site of the ATP binding protein. Some salts of alkaline earth metals, like magnesium chloride (MgCl₂), have a tendency to bind at the ATP binding site of respective protein, so that such salts are not preferred according to the present invention. On the other hand, e.g. alkali metal salts do not compete with the ATP binding site of ATP binding proteins. Consequently, particularly preferred salts are salts of alkali metals, especially sodium chloride (NaCl). The buffer used to separate the ATP binding protein(s) bound to the novel reagents (Kinator I, II III to V, VI to VIII and/or IX) from the proteins not bound also may contain high salt concentrations in the above-mentioned sense.

Using such specific conditions, i.e. high salt concentration, allows enriching of ATP binding proteins at least 10³-fold, preferably at least 10⁴-fold, and more preferably up to 10⁶-fold.

Besides enriching, with the medium and the method according to the present invention it is also possible to purify an ATP binding protein to a high degree, and vice versa, if one intends to deplete a pool of proteins specifically from ATP binding proteins, then this can also be achieved with the medium and the method according to the present invention.

The present invention also refers to a kit comprising at least one the mediums (compound of the classes A to K immobilized on a carrier) described in more detail above. The kit according the present invention may furthermore comprise one or more of the buffers described above.

In a further aspect the present invention refers to a method of making a quinazoline compound of the general formula (O) or a salt thereof:

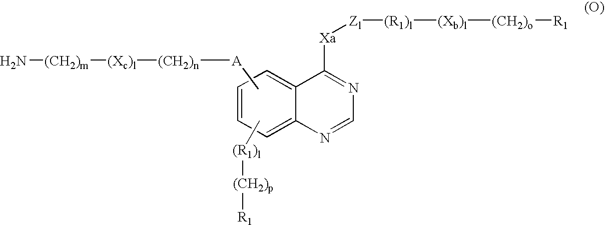

the method comprising the step (A):

reacting a compound with the general formula (Φ)

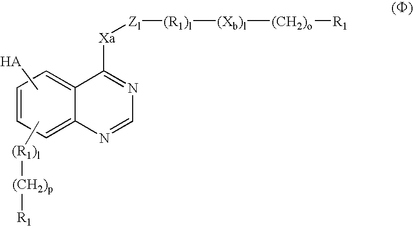

with a compound of the general formula (θ)

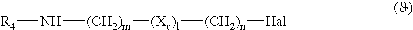

to give compound (K)

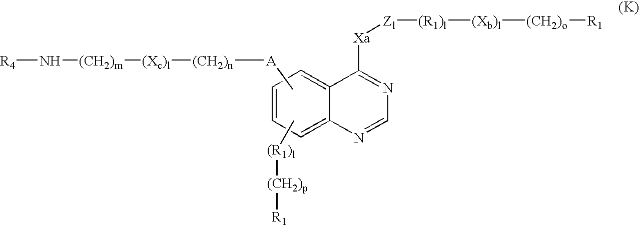

wherein the reaction is carried out in the presence of a base and an inert solvent,

and wherein

A is —O—, —S—, —NH—

Hal is —Cl, —Br, or —I;

Xₐ, X_{b}, and X_{c} are independently selected from Z, —CH₂—, —NH—, —O—, —S—,

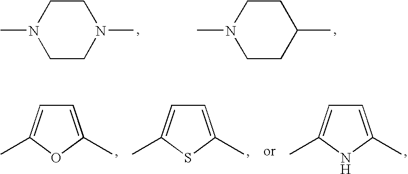

Z is —SO₂—NR₁—, —CO—, —O—CO—, —NH—CO—, —COO—, —CO—NH—, —CS—NH—, —OCH₂—, —SCH₂—, or —NH—CO—NH—,

1 is independently selected for each moiety to be 0 or 1,

each of m, n, o, and p is an integer independently selected for each moiety from 0 to 10 each R₁ is independently selected from —H, —O—, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or partially or fully substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),

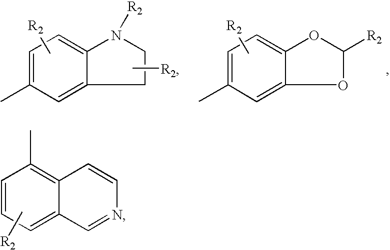

each R₂ is independently selected from —F, —Cl, —Br, —I, —CN, —OH, —SH, NH₂, C₁-C₆ alkyl (linear or branched), C₁-C₆-alkoxy, C₁-C₆-alkylthio, C₁-C₆-haloalkyloxy, partially or fully halogenated

C₁-C₆ alkyl (C₁-C₆-alkoxy denotes an O-alkyl group, C₁-C₆-alkylthio denotes an S-allyl group, C₁-C₆-haloalkyloxy denotes an halogen-alkyl-O group, C₁-C₆-haloalkyl denotes an halogen-alkyl group wherein the alkyl group is linear or branched),

R₄ is a leaving group, selected from the group consisting of t-butyloxycarbonyle (BOC), flourene-9-ylmethoxycarbonyle (Fmoc) or benzyloxycarbonyle and further comprising as step (B):

cleaving off the leaving group R4 to give compound (O) or a salt thereof.

In a preferred embodiment group A in compounds (O) and (Φ) is —NH—.

In a further preferred embodiment the base used in reaction step (A) is K₂CO₃ or Na₂CO₃ and the inert solvent is selected from the group consisting of acetonitrile, acetone, toluene, THF or DMF.

Reaction step (A) is preferably carried out under heating, preferably at a temperature at which the inert solvent refluxes.

It is preferred that in compounds (Φ), (θ) and (K),
1 is 0, o is 0, and p is 0
m is 0
n is an integer selected from 1 to 8, preferably from 2 to 6, and most preferably is 4,
Xa is —NH—,
R₃ is C₁-C₆ alkyl (linear or branched), and
R₁ is an unsubstituted or partially or fully substituted aryl, wherein the aryl is substituted by at least one of the substituents comprised in the group consisting of —F, —Cl, —Br, —I, —CN, —OH, —SH—, C₁-C₆-alkylthio, and benzyloxy-.

In a preferred embodiment of the invention compound ((Φ) is selected from the group consisting of

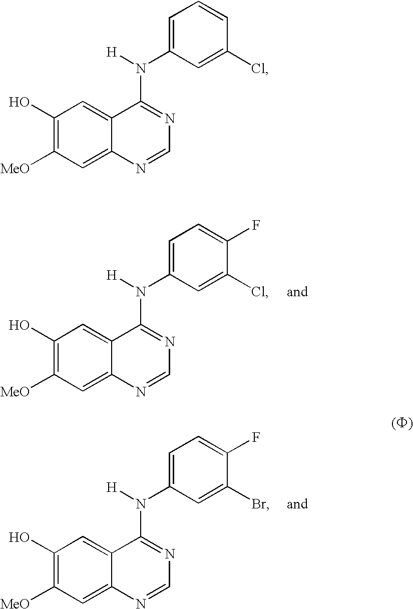

compound (θ) is

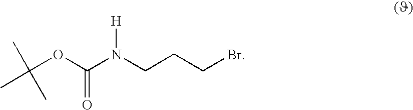

If the leaving group R₄ is the BOC moiety, it is preferred that this group cleaved of by reacting compound (K) with an protic acid, e.g. hydrochloric acid, in order to remove leaving group. Alternatively, compound (K) can be reacted with Me₃SiI is CHCl₃ or CH₃CN, or with AMCl₃ and PhOCH₃ in CH₂Cl₂. If R4 represents the Fmoc-group, this group can be removed by reacting compound (K) with a base selected from the group consisting of piperidine, morpholine or ethanolamine. If R4 represents benzyloxycarbonyle, this group can be removed by hydrogenation or reaction of compound (K) with Et3SiH with catalytic amounts of Et₃N and PdCl₂, Me3SiI in CH₃CN, AlC₃ and PhOCH₃ in CH₂Cl₂ or BBr₃ in CH₂Cl₂.

In a further aspect the invention relates to a method of making a compound with the general formula (Σ)

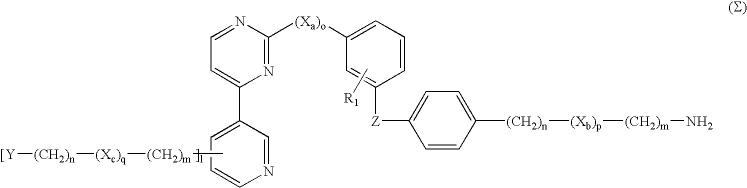

comprising the steps (A):

reacting a compound of the general formula (T)

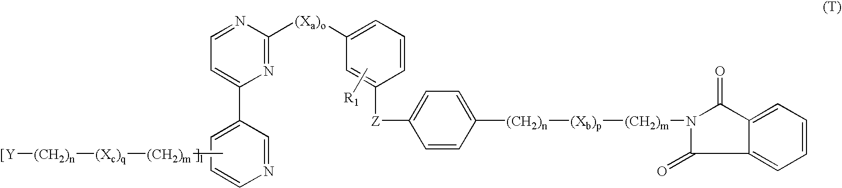

with hydrazine in a protic solvent and subsequently reacting the crude reaction product with an aqueous solution of an protonic acid, wherein in compounds (Σ) and (T)

Xₐ, X_{b} and X_{c} are independently selected from the group consisting of Z, —CH₂—, —NH—, —O—, —S—,

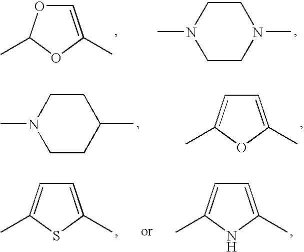

Y is —NH₂, —NHR₁, —OH, —SH or —SO(CH₃),

Z is —SO₂—NR₁, —CO—, —O—CO—, —NH—CO, —COO—, —CO—NH—, —OCH₂—, —SCH₂—,

1 is independently selected to be 0 or 1,

m is an integer independently selected from 0 to 10,

n is an integer independently selected from 0 to 10,

o is an integer independently selected from 0 to 10,

p is an integer independently selected from 0 to 10,

q is an integer independently selected from 0 to 10, and

R₁ is independently selected from —H, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), —F, —Cl, —Br, —I, —COOH, —NH₂,

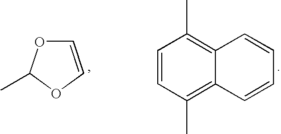

In a preferred embodiment, the protic solvent is selected from the group of alkyl alcohols, preferably from the group consisting of methanol, ethanol, propanol, iso-propanol, n-butanol and and iso-butanol, and most preferably is ethanol.

The protonic acid is preferably selected from hydrochloric acid or hydrobromic acid, and preferably is hydrochloric acid.

In a preferred embodiment, the method further comprises the step of providing the compound (T) by reaction of compound (Λ) or a salt thereof

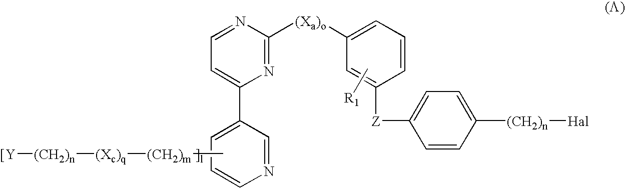

with compound (M)

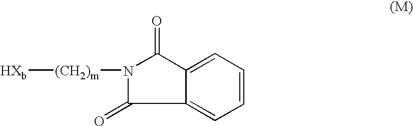

in the presence of a base,

wherein Hal is a halogen selected, preferably selected from the group consisting of Cl—, Br, and I—, and preferably is Br, and

wherein Xₐ, X_{b} and X_{c}, Y, Z, R₁, l, m, n, o, p, and q have the same meaning as in compounds (T) and (Z) as defined above.

The base is preferably selected from the group consisting of ammonia, primary amines, especially primary alkyl amines, secondary amines, especially secondary alkylamines or tertiary amines, especially tertiary alkylamines, and preferably is triethylamine.

The generic concept refers to the design of compounds which are kinase inhibitors, immobilized on a support material. These compounds are appropriate for kinase fishing. The design of these compounds is based on low-molecular weight kinase inhibitors with proven inhibitory potential towards a single, or an array of protein kinases. The molecular topology of the kinase inhibitors needs to rationalized in terms of pharmacophoric elements that facilitate high-affinity binding to the target enzymes. For that purpose, crystallographically determined structures, as well as homology structures of kinase-inhibitor complexes are structurally analysed by means of molecular modelling with the aim to discriminate the essential pharmacophoric groups from surface-accessible epitopes of the small molecule inhibitors. Once the surface accessible regions are identified, functional groups for further derivatization are introduced into the inhibitor structures in silico at various different positions. Docking in combination with molecular simulations assist in the final selection of the most appropriate derivatized novel analogue of a parent kinase inhibitor. Novel synthetic routes towards the derivatized kinase inhibitors are devised and ranked according to chemical feasibility. Once the final compound is successfully synthesized, retained kinase inhibition is checked and compared to the inhibitory profile of the parent compound.

### EXAMPLES

### Kinase Inhibitors Used and Their Immobilization

Protein kinase inhibitors used were compound A: 4-[4-(4-Fluoro-phenyl)-5-pyridine-4-yl-1H-imidazole-2-yl]-benzylamine (prepared as described in Gallagher et al., 1997, Bioorg. Med. Chem, 5, 49-64); compound B: 2-[4-(2-Amino-ethoxy)-phenylamino]-6-(2,6-dichloro-phenyl)-8-methyl-8H-pyrido[2,3-d]pyrimidine-7-one (prepared as described in Klutschko et al., 1998, J. Med. Chem., 41, 3276-3292), compound C: 3-[1-(3-Aminopropyl)-1H-indole-3-yl]-3-(1H-indole-3-yl) maleinimide, compound D: 3-[1-(3-Aminopropyl)-1H-indole-3-yl]-4-(1-methyl-1H-indole-3-yl) maleinimide, Compound E: 3-(8-Aminomethyl-6,7,8,9-tetrahydro-pyrido[1,2-a]-indole-10-yl)-4-(1-methyl-1H-indole-3-yl)-maleinimide (compound C, D and E each purchased from Calbio-chem), compound F: [6-(3-Amino-propoxy)-7-methoxy-quinazoline-4-yl]-(3-chloro-phenyl)-amine, compound G: 6-(3-Amino-propoxy)-7-methoxy-quinazoline-4-yl]-(3-chloro-4-fluoro-phenyl)-amine, compound H: 6-(3-Amino-propoxy)-7-methoxy-quinazoline-4-yl]-(3-bromo-phenyl)-amine, and compound I: 4-[4-(4-Amino-butyl)-piperazine-1-yl-methyl]-N-[4-methyl-3-(4-pyridine-3-yl-pyrimiidine-2-ylamino)-phenyl]-benzamide (compound F-I synthesized as described below).

1 g epoxy-activated Sepharose 6B (Amersham Biosciences) was swollen and washed twice in 50 ml H₂O and equilibrated to 50% DMF/0.1 M Na₂CO₃. Between all the washing steps, the Sepharose beads were spun down for 1 min at 2000 rpm in a desktop centrifuge and the supernatant was discarded. 300 μl drained beads were resuspended in 600 μl 20 mM compound A, C to I dissolved in 50% DMF/0.1 M Na₂CO₃. 1 μl 10 M NaOH was added followed by incubation overnight at 30° C. with continual agitation in the dark. After washing the beads three times in 1 ml 50% DMF/0.1 M Na₂CO₃ 600 μl 1 M ethanolamine were added to the drained beads and incubated for 6 h at 30° C. with permanent shaking in the dark. Finally the following washing steps were carried out in a volume of 1 ml each: First 50% DMF/0.1 M Na₂CO₃, then H₂O, then 0.1 M NaHCO₃ pH 8.0/0.5 M NaCl followed by 0.1 M NaAc pH 4.0/0.1 M NaCl and finally three times in buffer A (20 mM Hepes/NaOH pH 7.5, 150 mM NaCl, 0.25% Triton X-100, 1 mM EDTA, 1 mM EGTA). Compound B was coupled in the presence of 0.1M NaHCO₃ instead of 0.1 M Na₂CO₃ and the addition of 1 μl 19 M NaOH was omitted. The Kinator I to IX beads were stored in the dark at 4° C. as 1:1 suspension in buffer A plus 10 μg/ml aprotinin, 10 μg/ml leupeptin and 1 mM PMSF.

Synthesis of Compounds F-G

General method for the preparation of [3-(4-arylamino-7-methoxy-quinazoline-6-yloxy)-propyl]-carbamic acid tert-butyl ester derivatives:

1.00 mmol 7-Methoxyarylamino-quinazoline-6-ol derivative was refluxed in 20 cm³ acetonitrile with 0.26 g (1.10 mmol) N-BOC-3-propyl-bromide and 0.15 g (1.10 mmol) potassium carbonate for six to eight hours. The reaction mixture was cooled to room temperature, and the solvent was evaporated under reduced pressure. The residue was stirred in the mixture of 15 cm³ water and 15 cm³ ethyl acetate for half an hour, at 0° C. The product was filtered off, washed with 5 cm³ cold ethyl acetate, and air-dried.

General method for the preparation of 3-[4-(3-arylamino)-7-methoxy-quinazolin-6-yloxy]-propyl-ammonium chloride derivatives

0.50 mmol [3-(4-arylamino-7-methoxy-quinazolin-6-yloxy)-propyl]-carbamic acid tert-butyl ester derivative was suspended in 20 cm³ methanol, and 1.0 cm³ ethyl acetate saturated with hydrochloric acid was added into the reaction mixture. After stirring for two hours at room temperature, and half on hour at 0° C. the product was filtered off, was washed with 15 cm³ diethyl ether, and airdried.

### Kinator F

### {3-[4-(3-Chloro-phenylamino)-7-methoxy-quinazoline-6-yloxy)-propyl}-carbamic acid tert-butyl ester

Yield: 0.35 g (76%)

Rt: 2.84 min; Mol. Mass: 459

NMR, δ (ppm): 9.53 (s, 1H), 8.52 (s, 1H), 8.03 (s, 1H), 7.82 (m, 2H), 7.41 (t, 1H, J=8.07 Hz), 7.22 (s, 1H), 7.16 (dd, 1H, J²=7.91 Hz, J²=0.89 Hz), 6.91 (broad s, 1H), 4.18 (t, 2H, J=5.62 Hz), 3.95 (s, 3H), 3.15 (m, 2H), 1.96 (t, 2H, J=6.15 Hz), 1.38 (s, 9H).

### 3-[4-(3-Chloro-phenylamino)-7-methoxy-quinazolin-6-yloxy]-propyl-ammonium chloride

Yield: 0.17 g (85%)

Rt: 0.41, 0.69 min; Mol. Mass: 358

NMR, δ (ppm): 11.76 (s, 1H), 8.86 (s, 1H), 8.63 (s, 1H), 8.08 (broad s, 3H), 7.94 (t, 1H, J=1.87 Hz), 7.78 (d, 1H, J=8.16 Hz), 7.45 (m, 3H), 4.40 (t, 2H, J=6.00 Hz), 3.98 (s, 3H), 3.01 (m, 2H), 2.13 (m, 2H).

### Kinator G

### {3-[4-(3-Chloro-4-fluoro-phenylamino)-7-methoxy-quinazolin-6-yloxy]-propyl}-carbamic acid tert-butyl ester

Yield: 0.32 g (67%)

Rt: 2.87 min; Mol. Mass: 477

NMR, δ (ppm): 9.52 (s, 1H), 8.43 (s, 1H), 8.09 (dd, 1H, J¹=6.65 Hz, J²=2.2 Hz), 7.79 (m, 2H), 7.43 (t, 1H, J=9.12 Hz), 7.19 (s, 1H), 6.89 (broad s, 1H), 4.15 (t, 2H, J×5.68 Hz), 3.93 (s, 3H), 3.13 (m, 2H), 1.94 (t, 2H, J=6.13 Hz), 1.36 (s, 9H).

### 3-[4-(3-Chloro-4-fluoro-phenylamino)-7-methoxy-quinazolin-6-yloxy]-propyl-ammonium chloride

Yield: 0.18 g (88%)

Rt: 0.43, 0.73 min; Mol. Mass: 377

NMR, δ (ppm): 11.79 (s, 1H), 8.85 (s, 1H), 8.62 (s, 1H), 8.07 (m, 4H), 7.82 (m, 1H), 7.51 (t, 1H, J=9.09 Hz), 7.39 (s, 1H), 4.39 (t, 2H, J=6.03 Hz), 3.98 (s, 3H), 3.00 (m, 2H), 2.14 (m, 2H).

### Kinator H

### {3-[4-(3-Brom-phenylamino)-7-methoxy-quinazoline-6-yloxy]-propyl-carbamic acid tert-butyl ester

Yield: 0.41 g (82%)

Rt: 2.86 min; Mol. Mass: 503

NMR, δ (ppm): 9.50 (s, 1H), 8.50 (s, 1H), 8.12 (s, 1H), 7.85 (m, 2H), 7.30 (m, 3H), 6.89 (broad s, 1H), 4.16 (t, 2H, J=5.51 Hz), 3.93 (s, 3H), 3.13 (m, 2H), 1.93 (t, 2H, J=6.09 Hz), 1.36 (s, 9H).

### 3-[4-(3-Bromo-phenylamino)-7-methoxy-quinazolin-6-yloxy]-propyl-ammonium chloride

Yield: 0.11 g (52%)

Rt: 0.41, 0.85 min; Mol. Mass: 403

NMR, δ (ppm): 11.70 (s, 1H), 8.86 (s, 1H), 8.60 (s, 1H), 8.06 (broad s, 4H), 7.82 (d, 1H, J=7.71 Hz), 7.44 (m, 3H), 4.39 (t, 2H, J=5.82 Hz), 3.99 (s, 3H), 2.99 (m, 2H), 2.13 (m, 2H).

### Kinator I

### 4-{4-[4-(1,3-Dioxo-1,3-dihydro-isoindol-2-yl)-butyl]-piperazin-1-ylmethyl}-N-[4-methyl-3-(4-pyridin-3-yl-pyrimidin-2-ylamino)-phenyl]-benzamide

0.43 g (1.00 mmol) 4-Chloromethyl-N-[4-methyl-3-(4-pyridin-3-yl-pyrimidin-2-ylamino)-phenyl]-benzamide was refluxed in 50 cm³ acetonitrile with 0.36 g (1.00 mmol) 2-(4-Piperazin-1-yl-butyl)-isoindole-1,3-dione dihydrochloride and 0.61 g, 0.84 cm³ (6.04 mmol) triethylamine for six hours. The reaction mixture was cooled to room temperature, and the solvent was evaporated under reduced pressure. The residue was stirred in the mixture of 30 cm³ water and 30 cm³ chloroform for half an hour. The separated water phase was extracted with 30 cm³ chloroform, and the combined organic phase was washed with 30 cm³ water. The solvent was evaporated under reduced pressure. The crude product was crystallised from 20 cm³ acetonitrile to give title compound.

Yield: 0.29 g (43%)

Rt: 2.69 min; Mol. Mass: 680

NMR, δ (ppm): 10.15 (s, 1H), 9.27 (d, 1H, J=1.80 Hz), 8.97 (s, 1H), 8.68 (d, 1H, J=3.91 Hz), 8.49 (m, 2H), 8.08 (d, 1H, J=1.20 Hz), 7.85 (m, 6H), 7.45 (m, 5H), 7.20 (d, 1H, J=8.29 Hz), 3.57 (t, 2H), 3.50 (s, 2H), 2.34-2.22 (m, 10H), 1.59 (m, 2H), 1.42 (m, 2H).

### 4-[4-(4-Amino-butyl)-piperazine-1-ylmethyl]-N-[4-methyl-3-(4-pyridine-3-yl-pyrimidine-2-ylamino)-phenyl]-benzamide

0.29 g (0.42 mmol) 4-{4-[4-(1,3-Dioxo-1,3-dihydro-isoindol-2-yl)-butyl]-piperazine-1-ylmethyl}-N-[4-methyl-3-(4-pyridine-3-yl-pyrimidine-2-ylamino)-phenyl]-benzamide was stirred at room temperature in 50 cm³ ethanol with 0.13 g, 0.12 cm³ (2.56 mmol) hydrazine hydrate for two days. The solvent was evaporated under reduced pressure, and 20 cm³ of 1 M hydrochloric acid solution in water was added to the residue and stirred for ten minutes at room temperature. The insoluble material was filtered off, and the filtrate was basified with sodium hydrogen carbonate, and extracted three times with 30 cm³ chloroform. The combined organic phase was washed with 30 cm³ water. The solvent was evaporated under reduced pressure. The crude product was crystalised from 15 cm³ acetonitrile to give the title compound (Kinator I).

Yield: 0.13 g (57%)

Rt: 0.41, 1.00 min; Mol. Mass: 550

NMR, δ (ppm): 10.14 (s, 1H), 9.19 (s, 1H), 8.95 (s, 1H), 8.62 (d, 1H, J=3.50 Hz), 8.45 (m, 2H), 8.05 (s, 1H), 7.84 (d, 2H, J=8.04 Hz), 7.49 (m, 1H), 7.37 (m, 4H), 7.18 (d, 1H, J=8.32 Hz) 3.47 (s, 2H), 2.55, 2.48, 2.34, 2.18 (broad s, 12H), 1.37 (broad s, 4H).

Reagents and plasmids. Cell culture media and Lipofectamine were purchased from Invitrogen. Radiochemicals and epoxy-activated Sepharose 6B were from Amersham Biosciences. SB 203580 and histone H1 were from Merck. Compounds C, D and E were from Merck or Alexis. GST-ATF2 was obtained from Upstate. All other reagents were from Sigma.

A partial cDNA encoding amino acids 24 to 646 of GAK was PCR-amplified from human lung cDNA and inserted into vector pcDNA3 (Invitrogen) modified to attach a C-terminal VSV-G epitope (Kimura et al., 1997, Daub et al., 2002). GAK sequence encoding amino acids 26 to 392 was cloned into pGEX-4T1 for expression of recombinant GST fusion protein in E. coli.

The full length RICK coding sequence fused to a C-terminal hemagglutinin (HA) epitope tag was cloned into pPM7 expression vector (Inohara et al., 1998, Daub et al., 2002). Kinase-inactive K47R and inhibitor-insensitive T95M mutants were generated using a mutagenesis kit (Stratagene). Plasmids pPM7-RICK-dCst and pPM7-RICK-KRdCst express the 353 amino acid residues of wild-type or kinase-inactive RICK fused to a C-terminal streptag epitope. The expression cassette from pPM7-RICK-dCst was inserted into an adenovirus genome by recombination in bacteria as described (Daub et al., 2002).

Full length cDNAs encoding human SLK and adenosine kinase were PCR-amplified from HeLa cell cDNA and cloned into pRK-FLAG and pRK-myc expression plasmids (Sabourin et al., 2000, Spychala et al., 1996, Daub et al., 2002).

Cell culture and transfections. COS-7 and HeLa and HuH-7 cells were cultured in Dulbecco's modified Eagle's medium supplemented with 10% fetal bovine serum (FBS). COS-7 cells were transiently transfected as previously described (Daub et al., 2002). On the second day after transfection, cells were either lysed or phosphate-starved for a further 2 h in phosphate-free medium containing 10% dialysed FBS. Cells were then treated with inhibitor for 15 min and subsequently metabolically labelled with 70 μCi [³²P]orthophosphate for 30 min prior to cell lysis.

Cell lysis and in vitro association experiments. HeLa cells or transfected COS-7 cells were lysed in buffer containing 50 mM HEPES pH 7.5, 150 mM NaCl, 0.5% Triton X-100, 10% glycerol, 1 mM EDTA, 10 mM sodium pyrophosphate, 0.2 mM DTT plus additives (10 mM sodium fluoride, 1 mM orthovanadate, 10 μg/ml aprotinin, 10 μg/ml leupeptin, 1 mM phenylmethylsulfonyl fluoride). HuH-7 cells were lysed in 20 mM HEPES pH 7.5, 150 mM NaCl, 0.25% Triton X-100, 0.1 mM EDTA, 0.2 mM EGTA, 1 mM DTT plus additives. For some experiments, lysis was performed in 50 mM HEPES pH 7.5, 150 mM NaCl, 0.5% Triton X-100, 1 mM EDTA, 1 mM EGTA, 1 mM DTT plus additives. Lysates were pre-cleared by centrifugation and equilibrated to 1 M NaCl for in vitro association experiments. 25 μl drained Kinator I matrix or control matrix was incubated with 250 μl high salt lysate for 3 h at 4° C. Optionally, 2 mM free compound was added to the lysate. After washing with 2×500 μl lysis buffer without additives containing 1 M NaCl (high salt) and with 1×500 μl lysis buffer without additives containing 150 mM NaCl (low salt), the beads were eluted with 1.5×SDS sample buffer. To test different elution conditions for bound p38, beads were incubated in 100 μl low salt lysis buffer supplemented with 1 mM compound A or 10 mM ATP/20 mM MgCl₂ as indicated. HuH-7 cell lysates for testing of PKC binding was left at 150 mM NaCl for in-vitro association experiments. For precipitation of strep-tagged proteins, 250 μl lysate containing 150 mM NaCl were incubated with StrepTactin-MacroPrep beads (IBA) for 3 h at 4° C. Beads were then washed three times with the same buffer without additives. After SDS-PAGE, proteins were transferred to nitrocellulose membrane and immunoblotted with the indicated antibodies. Radioactively labelled RICK-KRdC was visaalised by autoradiography prior to detection with StrepTactin-HRP (IBA).

Affinity chromatography and preparative gel electrophoresis. 2.5×10⁹ frozen HeLa cells (4C Biotech) were lysed in 30 ml buffer containing 20 mM HEPES pH 7.5, 150 mM NaCl, 0.25% Triton X-100, 1 mM EDTA, 1 mM EGTA, 1 mM DTT plus additives (10 mM sodium fluoride, 1 mM orthovanadate, 10 μg/ml aprotin, 10 μg/ml leupeptin, 1 mM phenylmethylsulfonylfluoride, 10% glycerol), cleared by centrifugation and adjusted to 1 M NaCl. The filtrated lysate was loaded with a flow rate of 100 μl/min on a 12.5 mm×5 mm chromatography column containing 600 μl Kinator I or III matrix equilibrated to lysis buffer without additives containing 1 M NaCl. When using the Kinator II matrix, 40 g of pelleted HeLa cells were lysed and the extract was incubated in the presence of Kinator II matrix overnight prior to pouring the whole mixture into the chromatography column. The column was washed with 15 column volumes, equilibrated to lysis buffer without additives containing 150 mM NaCl and bound proteins were eluted in the same buffer containing 1 mM compound A, 10 mM ATP, 20 mM MgCl₂ with a flow rate of 50 μl/min. Proteins from Kinator II columns were eluted by several consecutive steps. The volume of protein-containing elution fractions was reduced to 1/10 in a SpeedVac concentrator prior to precipitation according to Wessel & Flugge (Wessel et al., 1984). Precipitated proteins were dissolved in 16-BAC sample buffer and after reduction/alkylation separated by two-dimensional 16-BAC/SDS-PAGE (Daub et al., 2002). Kinator II-purified proteins were resolved by two-dimensional IEF/SDS-PGE according to the manufacturer's instructions (Amersham). Coomassie stained spots were picked and subjected to analysis by mass spectrometry.

Mass spectrometry. Picked samples were destained in 30% Ethanol/10% acetic acid over night. Destained samples were washed twice in 0.1 M ammonium bicarbonate (NH₄HCO₃) and reduced with 10 mM DTT in 0.1 M NH₄HCO₃ for 30 min at 56° C. Samples were then dehydrated with acetonitril, rehydrated and alkylated with 55 mM Iodoacetamide in 0.1 M NH₄HCO₃ for 30 min in the dark and washed twice with 0.1 M NH₄HCO₃. Dried samples were reswollen in trypsin (Promega) solution containing 50 mM NH₄HCO₃/10% acetonitrile and digested overnight at 37° C. Peptides were washed out once with 50 mM NH₄HCO₃ and twice with 5% formic acid. Guanidination for MALDI mass mapping was performed as described (Beardsley et al., 2002). Sample clean up was performed onto ZipTips using the manufacturer's standard procedures (Millipore).

MALDI spectra were acquired using a Bruker Ultraflex TOF/TOF mass spectrometer with LIFT technology and anchor chip targets. Data analysis was performed using Bruker's Biotools and the Mascot program. Searches were done against the NCBI database.

In vitro kinase assays. Kinase reactions were performed for either 10 min (p38α, JNK1, JNK2, CK1δ) or 30 min (RICK, GAK) at 30° C. in a total volume of 50 μl. All kinases were assayed in 50 mM Tris-HCl pH 7.5, 10 mM MgCl₂, 1 mM DTT, 0.1 mM EGTA, 100 μM ATP and [γ-³²P]ATP in the presence of indicated SB 203580 concentrations. When compound B was tested, 50 μM ATP were included In addition, JNK1 and JNK2 assays were performed in the presence of 2 μM ATP. Kinase substrate proteins included were 0.4 mg/ml myelin basic protein (p38α, RICK), 0.4 mg/ml casein (CK1δ), 0.2 mg/ml histone H1 (GAK) and 0.1 mg/ml GST-ATF2 (JNK1, JNK2). Abl kinase assays were performed for 30 min in 10 mM Tris-HCl pH 7.5, 25 mM MgCl₂, 5 mM MnCl₂, 0.51 mM DTT, 0.5 mM EGTA, 0.05 mM orthovanadate, 50 μM ATP and [γ-³²P]ATP in the presence of indicated compound B concentrations. N-terminally FLAG-tagged SLK was transiently expressed in COS-7 cells and immunoprecipitated with 3 μg M2-FLAG for 3 h at 4° C. After binding, the beads were washed 3 times with 500 μl 1× Triton-Lysis buffer and 1 time with 500 μl 1× kinase buffer (20 mM Hepes pH 7.5, 15 mM MgCl₂, 80 mM KCl, 1 mM Na₂VO₄ and 0.1 M DTT). The linase assay was performed in a total volume of 60 μl. To 15 μl drained protein-G-Sepharose beads 34 μl Kinasebuffer and 1 μl of 50% DMSO or compound C(50% DMSO, several concentrations) were added and incubated for 10 min at 4° C. The reaction was started by adding 10 μl containing 100 μM ATP, 1 μCi [γ-³²P] ATP and 20 μg MBP and stopped with 25 μl 3× Laemmli-SDS-buffer after 10 min. Measurement of Cdk2 activity was assayed according to the supplier's protocol (Upstate). Reactions were stopped by addition of 3× SDS sample buffer. After gel electrophoresis, phosphorylated substrate proteins were visualised by autoradiography and quantified by phosphorimaging. Determination of IC₅₀ [0-100%] values was performed using GraFit (Erithacus).

Kᵢ determination of compound C for human oxidoreductase: Enzyme activity and inhibition were determined spectrophotometrically (Spectramax Plus384, Molecular Devices) by measuring the reduction of 3-(4,5-dimethylthizaol-2-yl)-2,5-diphenyltetrazolin (MTT) at 610 nm and 30° C. In this assay we used NADH (Roche) as electron donor for the menadion reduction and MTT for the continuous reoxidation of menadiol. The reactions (200 μl) were performed in 96 well plates, containing 50 mM KₓHₓPO₄ pH 7.5, 1 μl NQO2 (XY units), 40 μM Menadion, 200 μM MTT and increasing concentration of NADH (0-1000 μM) in the presence of compound C (0, 1, 30, 60 μM). Using a Lineweaver-Burk application we determined the apparent Kₘ values for the different compound C concentrations. By plotting the different K_{m, app} against its corresponding compound C concentrations we calculated the Kᵢ.

Results

The invention relates to the generation of several new chromatography media for the purpose of affinity purification of cellular kinases. These chromatography media are referred to as “Kinator matrices”. In the following section, various lines of evidence for the functionality of the Kinator matrices are provided. Those include efficient purification of known and previously unknown targets including both linase and non kinase targets of the immobilized compounds, their identification by mass spectrometry analysis, the validation of specific interaction with Kinator beads by immunoblot analysis and both in vitro and in vivo enzyme activity assays for verification of their sensitivity to inhibition by the respective immobilized compounds or structurally similar compounds.

The mitogen-activated protein kinase p38 was originally identified as the major cellular target of anti-inflammatory drugs such as SB 203580, which belong to the pyridinyl imidazole class of compounds (Cuenda et al., 1995, Lee et al. 1994) (FIG. 1a). The crystal structure of p38 in complex with SB 203580 shows exposure of the inhibitor's sulfoxide moiety at the protein surface, suggesting a suitable site for the attachment of linkers extending from solid support material (Tong et al., 1997). For the purpose of immobilisation, a closely related derivative of SB 203580 was selected, possessing a primary methylamine function instead of the sulfoxide moiety at the accessible position. This inhibitor is referred to as compound A (FIG. 1a) (Gallagher et al., 1997). Compound A was pegylated to yield compound Apeg, resulting in a structure similar to compound A covalently coupled to epoxy-activated Sepharose (FIG. 1a). SB 203580, compound A and compound Apeg were then tested in kinase assays using recombinant p38α as enzyme and myelin basic protein as substrate (FIG. 1b). In agreement with published data, SB 203580 and compound A inhibited p38 activity with IC₅₀ values of about 40 nM and 4 nM, respectively (Gallagher et al., 1997, Davies et al., 2000). Importantly, extension of compound A by pegylation reduced its inhibitory effect on p38 by one order of magnitude, but even so, compound Apeg was as effective in inhibiting p38 as SB203580 (FIG. 1b). It is therefore concluded that the interaction of compound A with p38 should be retained after immobilisation of compound A on epoxy-activated Sepharose to generate the Kinator I matrix (FIG. 1a). This assumption could be confirmed by the in vitro association experiment shown in FIG. 1c, in which p38 from HeLa total cell lysate specifically bound to the Kinator I matrix, but not to control beads devoid of covalently coupled compound A. However, when the batch purification was performed at a physiological ionic strength of 150 mM NaCl, parallel silver staining showed a variety of bound proteins, indicating only moderate p38 enrichment. Testing of different buffer compositions led to the identification of high salt concentrations of 1 M NaCl as critical for efficient retention and separation of p38 from the vast majority of cellular proteins (FIG. 1c). Furthermore, elution under non-denaturing conditions turned out to be far more efficient when both compound A and ATP were included in the elution buffer rather than either component alone (FIG. 1d). Employing these optimised adsorption and elution conditions, an affinity chromatography method on Kinator I matrix-containing columns using total cell extracts from 2.5×10⁹ HeLa cells as starting material was established. As shown by immunoblotting, the MAP kinase p38 was nearly quantitatively bound to the Kinator I matrix, while the general protein pattern detectable in the initial extract reappeared in the flow-through. After extensive washing, p38 was efficiently eluted in the presence of both ATP and compound A (FIG. 2a, lower panel). Remarkably, at this level of sensitivity no protein bands could be visualised in the elution fraction (FIG. 2a, upper panel), demonstrating a substantial enrichment, which was at least 10000-fold as shown by comparison of sample and flow-through with increasing aliquots of the fraction containing several retained proteins that had been specifically eluted from the column (FIG. 2b). According to enhanced specificity a larger amount of cell lysate used as the starting material could be loaded onto the Kinator affinity chromatography column, resulting in the ability to detect also kinases of lower abundance. Proteins binding with high affinity to the Kinator matrix could be enriched by this method up to 10⁶-fold. Silver staining revealed several bands, indicating the purification of various other proteins in addition to p38 on the Kinator I affinity matrix. To identify these proteins, they were first resolved by preparative gel electrophoresis in the presence of the cationic detergent 16-benzyldimethyl-n-hexadecylammonium chloride (16-BAC) followed by standard SDS-PAGE in the second dimension (Daub et al., 2002). Coomassie staining of 16-BAC/SDS gels visualised about 30 protein spots, which were excised and analysed by mass spectrometry (FIG. 2c). In addition to p38, which was represented by one of the most prominent spots, a variety of additional protein kinases could be identified. These included c-jun N-terminal kinase (JNK) isoforms and glycogen synthase kinase 3β (GSK3β), kinases known to be affected by micromolar concentrations of SB 203580 (Whitmarsh et al., 1997, Davies et al., 2000). In addition to these known interactors of SB 203580, various other protein kinases were identified. These included RICK (Rip-like interacting CLARP kinase/Rip2/CARDLAK), different CK1 isoforms, cyclin G-associated kinase (GAK), protein kinase Nβ (PKNβ) and janus linase 1 (JAK1). These results demonstrate the efficient purification of various cellular protein kinases employing the Kinator I matrix. To confirm the mass spectrometry results, total lysates from HeLa cells or COS-7 cells expressing an epitope-tagged GAK(24-646) fragment were subjected to in vitro association with either control beads or Kinator I beads with or without free compound A present in the lysate. Both unbound proteins from the supernatants and proteins retained on the beads were immunoblotted with specific antibodies for JNK, GSK3, RICK, CK1α, CK1ε or for the epitope tag of GAK(24-646). All endogenous kinases from HeLa cells and the truncated GAK specifically interacted with the Kinator I matrix, thus verifying the identifications by mass spectrometry (FIG. 2d). Interestingly, GSK3 binding was specific for the β isoform, with no significant interaction observed for GSK3α.

Association with the Kinator I matrix did not provide a quantitative measure for the potency of pyridinyl imidazoles such as SB 203580 towards the specifically bound protein kinases. To test how binding might translate into inhibitor sensitivity, the effect of SB 203580, a widely used kinase inhibitor structurally similar to compound A, on the in vitro kinase activities of p38α, RICK, GAK and CK1δ in the presence of 100 μM cold ATP was analysed. SB 203580 inhibited recombinant p38α in the tested assays with an IC₅₀ value of 38 nM, in good agreement with published data (FIG. 3a) (Davies et al., 2000). RICK was even more potently inhibited by SB 203580 than p38α with an IC₅₀ value of only 16 nM. Moreover, the inhibitor concentrations required to inhibit CK1δ and GAK kinase activities by 50% were only about three-fold higher than that determined for p38α (FIG. 3a). These data demonstrate that low concentrations of SB 203580 inhibit several protein kinases. JNK1 was also efficiently retained by the Kinator I matrix, although this kinase is not inhibited by 10 μM SB 203580 in vitro (Davies et al., 2000). To resolve this issue, kinase assays with both JNK1 and JNK2 at either 100 μM ATP, the standard concentration used, or at only 2 μM ATP, which resembles the conditions during affinity purification under which cell-derived Mg²⁺-ATP cannot compete for binding due to the presence of EDTA as a chelating reagent were performed. As shown in FIG. 3b, the SB 203580 concentrations required for half maximal kinase inhibition significantly dropped from more than 100 μM to 13 μM for JNK1 and from 11 μM to 0.7 μM for JNK2 upon reduction of the ATP concentration to 2 μM, thereby demonstrating that the Kinator I matrix can be employed for purification of low affinity targets of pyridinyl imidazole inhibitors. Finally, the highly SB 203580-sensitive serine(threonine kinase RICK was chosen to analyse how the enzymatic activity of one of the new inhibitor targets is affected upon SB 203580 treatment in intact cells. As a cellular linase substrate, a kinase-dead fragment of RICK (RICK-KRdC) was expressed, which was heavily phosphorylated upon co-transfection of the catalytically active, full-length kinase (FIG. 3c). RICK-mediated substrate phosphorylation was inhibited by SB 203580 in a dose-dependent manner, with 0.3 μM of compound already conferring about 50% inhibition. Interesgly, RICK possesses a conserved threonine residue equivalent to Thr-106 of p38, which is critical for inhibitor binding and was shown to render p38 resistant to SB 203580 when mutated to a larger amino acid (Tong et al., 1997, Eyers et al., 1998). This structural determinant is equally important for RICK, as mutation of the corresponding Thr-95 to methionine rendered RICK resistant to SB 203580 at all concentrations tested, further demonstrating that the inhibitor directly affected RICK kinase activity in intact cells (FIG. 3c). With the notable exception of CK1, the critical threonine residue is conserved in all kinases found to be potently inhibited by SB 203580, although about 75% of all protein kinases possess a larger hydrophobic residue in this position (FIG. 3d).

The pyrido[2,3-d]pyrimidine derivative compound B (2-[4-(2-amino-ethoxy)-phenylamino]-6-(2,6-dichloro-phenyl)-8-methyl-8H-pyrido[2,3-d] pyrimidine-7-one) was covalently coupled to epoxy-activated Sepharose to generate the Kinator II matrix (FIG. 4). Compound B has been described as potent inhibitor of Src and fibroblast growth factor receptor 1 (FGFR1) tyrosine kinases with IC₅₀ values of 55 nM and 17 nM (Klutschko et al., 1998). In addition, structurally related compounds such as PD180970 potently inhibit BCR-ABL kinase activity (Wisniewski et al., 1998). Abl kinase activity was also inhibited by compound B with an IC₅₀ value of 1.4 nM. To verify the functionality of the Kinator II matrix, lysate from FGFR1-overexpressing COS-7 cells or HeLa cell lysates were subjected to in-vitro association experiments with either control beads or Kinator II beads with or without free compound B present in the lysate. Both unbound proteins from the supernatants and proteins retained on the beads were immunoblotted with specific antibodies against FGFR1, Abl or Src. As shown in FIGS. 5A-B, all three kinases specifically associated with Kinator II beads in the absence of added compound B but not when it was included in the lysate. 40 g of pelleted HeLa cells were then lysed and the extract in the presence of Kinator II matrix was incubated overnight prior to pouring the whole mixture into a chromatography column. The flow-through was collected and precipitated with 55% ammonium sulfate. After washing of the Kinator II column, bound proteins were eluted by 4 consecutive elution steps. For the first elution step, 5 mM ATP/20 mM MgCl₂ were included in the running buffer. For the second elution step, 1 mM compound B was added. For the third elution step, both 5 mM ATP/20 mM MgCl₂ and 1 mM compound B were added. The fourth elution was performed in the presence of 7 M urea/2 M thiourea. The precipitated initial flow-through was dialysed against running buffer and then reapplied onto the Kinator II column. Bound proteins were first eluted with 5 mM ATP/20 mM MgCl₂ and subsequently eluted with 7 M urea/2 M thiourea. Proteins in all six eluate fractions were precipitatated and then resolved by two-dimensional gel electrophoresis (FIGS. 6A-F). Stainable protein spots were then excised and analysed by mass spectrometry. Identified protein spots are indicated on the 2D gels (FIGS. 6A-F). In parallel, Kinator II-purified proteins were resolved by one-dimensional SDS-PAGE prior to excision of protein bands and mass spectrometry. The following novel protein kinase targets of Kinator II were identified: p38α, RICK (Riplike interacting CLARP kinase/Rip2/CARDIAK), GAK (cyclin G-associated kinase), CK1α, δ and ε, GSK3α, GSK3β, Weel, EphB4, Yes, Csk, Aurora A, AMPKβ, JNK1, JNK2, JNK3, ERK1, ERK2, MEK1, MEX2, TAK1, ACK, RSK1, RSK2, MST4, NEK2, ZAK and STK24. As shown in FIG. 7 for a selection of the identified protein kinase targets, specific association with Kinator II beads was detected in the absence of added compound B but not when it was included in the lysate. To test how binding might translate into inhibitor sensitivity, the effect of compound B on the in vitro kinase activities of p38α, RICK, GAK in the presence of 50 μM cold ATP, a concentration which allows comparisons of these results with previously published data (Klutschko et al., 1998) was analysed. Compound B inhibited recombinant p38α, RICK and GAK with IC₅₀ values of 5.8 nM, 0.16 nM and 2.7 nM, respectively (FIG. 8). These results demonstrate the utility of the Kinator II matrix for identification of cellular protein kinases highly sensitive to pyrido[2,3-d]pyrimidine inhibitors as compound B. These results further show the identification and characterization of serine/threonine protein kinases as potently inhibited cellular targets of a compound previously assumed to be specific for protein tyrosine kinases.

In comparison, the inhibitor concentrations required to inhibit CK1δ, JNK1 and JNK2 kinase activities by 50% were 7.2 μM, 2.4 μM and 3.8 μM, about three to four orders of magnitude higher than for p38α, RICK and GAK. Thus, the Kinator II matrix is suitable for isolation of cellular protein kinases with either high affinities for compound B as shown for p38α, RICK and GAK or significantly lower affinities as determined for CK1δ, JNK1 and JNK2. The presence of a small side chain such as threonine in the conserved positions corresponding to Thr-106 of p38α closely correlated with sensitivity of the tested protein kinases for inhibition by compound B, in agreement with GAK and RICK also possessing a threonine and CK1δ, JNK1 and JNK2 possessing a more space-filling methionine residue at this site. To demonstrate that Kinator II-isolated protein kinases are targeted by compound B in vivo, we performed cellular assays for p38 and RICK activity. When HeLa cells were pretreated with the indicated concentrations of compound B for 15 min prior to 30 min stimulation with 10 μg/ml anisomycin to activate p38, p38-mediated MAPKAP kinase-2 phosphorylation was inhibited in a dose-dependent manner with a cellular IC₅₀ in the low nanomolar range (FIG. 9A, upper panel). As a control, EGF-stimulated Rsk1 phosphorylation was not significantly affected upon treatment of intact cells with compound B (FIG. 9B, lower panel). Furthermore, phosphorylation of kinase-dead fragment of RICK (RICK-KRdC) upon co-transfection of the catalytically active, full-length kinase was strongly inhibited by 100 nM and abrogated by 1000 nM compound B in intact cells. Thus, these data demonstrate the utility of the Kinator II matrix employing immobilized compound B for identifying novel targets linked to previously unrecognized potential therapeutic applications such as inflammation for p38 and RICK protein kinases identified here.

Protein kinase inhibitors belonging to the bisindolylmaleinimide class of compounds were originally characterized as potent PKC (protein kinase C) blockers. More recent evaluations of their specificities revealed additional kinase targets of this compound class such as Rsk1 and GSK (Davies et al., 2000). The bisindolylmaleinimide compounds C, D and E were immobilized on Sepharose beads to generate the novel Kinator III, IV and V matrices, respectively, for the purpose of affinity purification of cellular target enzymes.

To test whether immobilized compound C retained its ability to interact with PKCα after immobilisation, total cell lysates were either incubated with control beads or Kinator III beads in the absence or presence PKC-specific cofactors (100 μg/ml phosphatidylserine, 20 μg/ml diacylglycerol and 300 μM CaCl₂). Interestingly, only when PKCα had been transferred into an active state due to co-factor addition, a strong and specific interaction with the Kinator III beads correlating with PKCα depletion from the respective supernatant fraction was observed, indicating that the Kinator III matrix can discern between active and inactive protein kinases as found for PKCα (FIG. 10A). Similar observations were made for Rsk1, whose activation upon EGF stimulation of intact cells correlated with induced binding to Kinator III beads, as revealed by the time course experiment shown in FIG. 10B. Affinity chromatography on Kinator III matrix-containing columns was then performed using total cell extracts from 2.5×10⁹ HeLa cells as starting material. After sample loading and extensive washing, specifically bound proteins were eluted with running buffer containing both free compound C and 10 mM ATP and then precipitated prior to separation by 16-BAC/SDS-PAGE (FIG. 11). Coomassie-stainable spots were excised and analyzed by mass spectrometry. Besides known interaction partners of compound C, Ste20-like kinase (SLK) was identified as a novel protein kinase target. For characterization of SLK, full length human enzyme containing an N-terminal FLAG epitope was expressed in COS-7 cells and isolated by immunoprecipitation. In vitro kinase assays revealed potent inhibition of SLK activity by compound C with an IC₅₀ value of around 150 nM (FIG. 12A). Interestingly, two of the most prominent spots isolated with Kinator III beads were identified as adenosine linase and quinone reductase 2 (HQO2), two enzymes not belonging to the protein kinase family. As shown in FIG. 12B, transiently expressed adenosine kinase specifically associated with Kinator III beads. Furthermore, adenosine linase activity was reduced by about 50% in the presence of 1 μM compound C. For quinone reductase 2, in vitro measurements of enzymatic activity indicated a dose dependent inhibition by compound C with an IC₅₀ value of about 16 μM (FIG. 12C). Next, the cellular protein binding to the related Kinator III, IV and V matrices was compared. As seen on the silver-stained gel in FIG. 13, some protein bands were found to be specifically associated with all three affinity matrices, whereas other proteins bound to only one or two of them. Immunoblot analysis of some kinases revealed PKCα, PKCε and GSK3 as common interaction partners, whereas Rsk1 and Cdk2 were preferentially recruited to Kinator V beads. Stronger binding of Cdk2 to Kinator V beads could further be correlated with a more potent inhibition of its in vitro kinase activity by free compound E compared to compounds C and D. (FIG. 14). Thus, increased binding to Kinator beads was found to translate into higher sensitivity for the respective compound that was immobilized on the respective Kinator matrix.

The three quinazoline compounds F, G and H and the phenylamino pyrimidine compound I were synthesized and immobilized on epoxy-activated Sepharose to generate the Kinator matrices VI, VII and VIII and IX, respectively. As shown by immunoblot analysis, all four Kinator materials specifically bound the epidermal growth factor receptor (EGFR) and the serine/threonine kinase RICK (FIG. 15, lower panel). Moreover, each of these materials bound various cellular proteins not found to be associated with control beads devoid of immobilized compound, thereby demonstrating the functionality of these Kinator matrices. In summary, a variety of novel affinity reagents were developed according to structural criteria of protein kinase-kinase inhibitor complex formation and established their functionality for the purpose of efficient protein kinase enrichment Different Kinator matrices were useful for the purification of different, partially overlapping sets of cellular protein kinases. Various examples were given to demonstrate that kinase binding to Kinator matrices correlated with inhibition of kinase activity by the free, corresponding inhibitor in vitro and in intact cells.

In Table 1 below, the compounds used to isolate protein kinases from a pool of proteins are given with their names and structural formulas.

TABLE 1
Compounds A to I (Kinators I to IX)

| Compound | Name | Structure |
|---|---|---|
| A | 4-[4-(4-Fluoro- phenyl)-5-pyridine- 4-yl-1H-imidazole- 2-y1]-benzylamine | 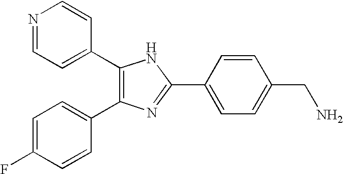 |
| B | 2-[4-(2-Amino- ethoxy)- phenylamino]-6- (2,6-dichloro- phenyl)-8-methyl- 8H-pyido[2,3-d]pyrimidine-7-one | 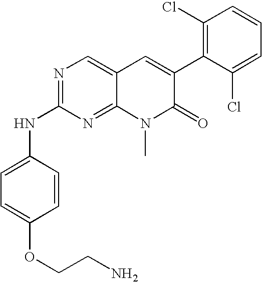 |
| C | 3-[1-(3- Aminopropyl)-1H- indole-3-yl]-4-(1H- indole-3-yl) maleinimide | 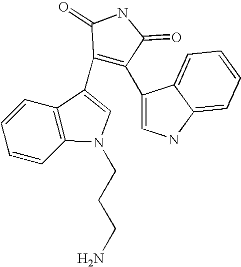 |
| D | 3-[1-(3- Aminopropyl)-1H- indole-3-yl]-4-(1- methyl-1H-indole -3-yl)maleinimide | 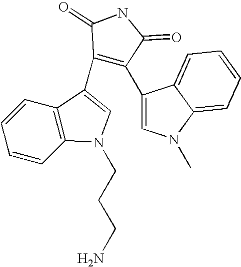 |
| E | 3-(8-Aminomethyl- 6,7,8,9-tetrahydro- pyrido[1,2-a]- indole-10-yl)-4-(1- methyl-1H-indole-3 -yl)-maleinimide | 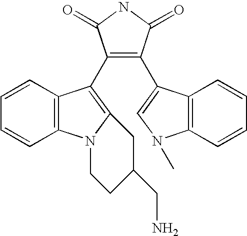 |
| F | [6-(3-Amino- propoxy)-7- methoxy- quinazoline-4-yl](3-chloro-phenyl)- amine | 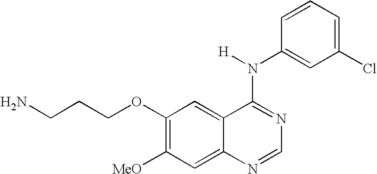 |
| G | 6-(3-Amino- propoxy)-7- methoxy- quinazoline-4-yl]- (3-chloro-4-fluoro- phenyl)-amine | 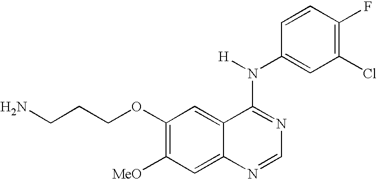 |
| H | 6-(3-Amino- propoxy)-7- methoxy- quinazoline-4-yl]- (3-bromo-phenyl)- amine | 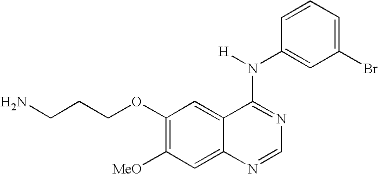 |
| I | 4-[4-(4-Amino- butyl)-piperazine-1- yl-methyl[-N-[4- methyl-3-(4- pyridine-3-yl- pyrimidine-2- ylamino)-phenyl]- benzamide | 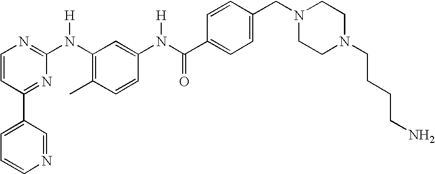 |

### REFERENCES

Beardsley, R. L. & Reilly, J. P. Optimization of guanidination procedures for MALDI mass mapping. Anal. Chem. 74, 1884-1890 (2002).
Cuenda, A. et al. SB 203580 is a specific inhibitor of a MAP kinase homologue which is stimulated by cellular stresses and interleukin-1. FEBS Lett. 364, 229-233 (1995).
Daub, H. et al. Identification of SRPK1 and SRPK2 as the major cellular kinases phosphorylating hepatitis B virus core protein. J. Virol. 76, 8124-8137 (2002).
Davies, S. P., Reddy, H., Caivano, M. & Cohen, P. Specificity and mechanism of action of some commonly used protein kinase inhibitors. Biochem. J. 351, 95-105 (2000).
Eyers, P. A., Craxton, M., Morrice, N., Cohen, P. & Goedert, M. Conversion of SB 203580-insensitive MAP linase family members to drug-sensitive forms by a single amino-acid substitution. Chem. Biol. 5, 321-328 (1998).
Gallagher, T. F. et al. Regulation of stress-induced cytokine production by pyridinylimidazole; Inhibition of CSBP kinase. Bioorg. Med. Chem. 5, 49-64 (1997).
Inohara, N., del Peso, L., Koseki, T., Chen, S. & Nunez, G. RICK, a novel protein kinase containing a caspase recruitment domain, interacts with CLARP and regulates CD95-mediated apoptosis. J. Biol. Chem. 273, 12296-12300 (1998).
Kimura, S. H., Tsuruga, H., Yabuta, N., Endo, Y. & Nojima, H. Structure, expression, and chromosomal localization of human GAK. Genomics 44, 179-187 (1997).
Klutschko, S. R. et al. 3-substituted Aminopyrido[2,3-d]pyrimidin-7(8H)-ones. Structure-activity relationships against selected tyrosine kinases and in vitro and in vivo anticancer activity J. Med. Chem. 41, 3276-3292 (1998).
Lee, J. C. et al. A protein kinase involved in the regulation of inflammatory cytokine synthesis. Nature 372, 739-746 (1994).
Sabourin, L. A., Tamai, K., Seale, P., Wagner, J. & Rudnicki, M. A. Caspase 3 cleavage of the Ste20-related kinase SLK releases and activates an apoptosis-inducing kinase domain and an actin-disassembling region. Mol. Cell. Biol. 20, 684-696 (2000).
Spychala, J., Datta, N. S., Takabayashi, K., Datta, M., Fox, I. H., Gribbin, T. & Mitchell, B. S. Cloning of human adenosine kinase cDNA: sequence similarity to microbial ribokinases and fructokinases. Proc. Natl. Acad. Sci. U.S.A. 93, 1232-1237 (1996).
Tong, L. et al. A highly specific inhibitor of human p38 MAP linase binds in the ATP pocket. Nat. Struct. Biol. 4, 311-316 (1997).
Wessel, D. & Flügge, U. I. A method for quantitative recovery of protein in dilute solutions in the presence of detergents and lipids. Anal. Biochem. 138, 141-143 (1984).
Whitmarsh, A. J., Yang, S.-H., Su, M. S., Sharrocks, A. D. & Davis, R. J. Role of p38 and JNK mitogen-activated protein kinases in the activation of ternary complex factors. Mol. Cell. Biol. 17, 2360-2371 (1997).
Wisniewski, D. et al. Characterisation of potent inhibitors of the Bcr-Abl and the c-kit receptor tyrosine kinases. Cancer Res. 62, 4244-4255 (2002).

### DESCRIPTION OF FIGURES

FIG. 1. Generation of a functional Kinator I matrix. (A) Chemical structure of SB 203580 compared with compound A in its free, pegylated and immobilised form. (B) In vitro kinase reactions were performed with recombinant p38α enzyme using MBP as a substrate. The pyridinylimidazoles SB 203580, compound A and compound Apeg were added at the indicated concentrations. After SDS-PAGE, MBP phosphorylation was detected by autoradiography and quantified by phosphorimaging. Kinase activitiy in the absence of inhibitor was set to 100% and remaining activities at different inhibitor concentrations are expressed relative to this value. (C) Total lysates from HeLa cells were subjected to in vitro associations with either control or Kinator I matrix. Incubations were performed in the presence of either low or high NaCl concentrations of 150 mM or 1 M, respectively. Relative to the total cell lysate, 100× aliquots of the bound protein fractions were resolved by gel electrophoresis for subsequent silver staining (upper panel) and in parallel 5× aliquots were analysed by immunoblotting with p38-specific antibody (lower panel). (D) Cellular p38 bound to Kinator I beads was eluted with lysis buffer supplemented with 1 mM compound A or 10 mM ATP/20 mM MgCl₂ as indicated. For comparison, quantitative p38 elution was performed with SDS sample buffer. After gel electrophoresis, eluted p38 was detected by immunoblotting.

FIG. 2: Efficient afnity purification of protein kinases specifically targeted by immobilised p38 inhibitor. HeLa whole cell lysate was subjected to Kinator I affinity chromatography and the bound proteins were eluted with a combination of ATP and free compound A. (A) After gel electrophoresis and transfer onto nitrocellulose membrane, cell lysate, flow-through, wash and elution fractions were analysed by Ponceau S staing and subsequent immunoblotting using specific anti-p38 antibody. (B) Increasing aliquots of pooled fractions containing protein specifically eluted from the Kinator I column were visualised in comparison to cell lysate, flow-through and wash fractions. The silver stained SDS-polyacrylamide gel demonstrates at least 5000-fold enrichment of retained proteins. (C) The large remainder of the proteins purified by affinity chromatography on the Kinator I matrix was separated by 16-BAC/SDS-PAGE and stained with Coomassie blue. The indicated spots (arrows) were analysed by mass spectrometry. Spots containing identified protein kinases are marked by their names. (D) Results from mass spectrometry analysis were confirmed by in vitro association of total lysates from HeLa cells or COS-7 cells expressing truncated GAK(24-646) protein fused to a C-terminal vesicular stomatitis virus G protein (VSV-G) epitope with either control matrix or Kinator I matrix. Free compound A was present in the lysate where indicated. Both non bound proteins from the supernatants and bound proteins eluted from the matrix were immunoblotted with specific antibodies for GSK3α/β, JNK, RICK, CK1α, CK1ε and for the VSV-tag of GAK(24-646)-VSV-G.

FIG. 3. Characterisation of protein kinases inhibited by SB 203580. (A) The concentration-dependent inhibition of p38α, RICK, CK1δ and GAK by SB 203580 was determined. Kinase activities in the absence of inhibitor were set to 100% and remaining activities at different SB 203580 concentrations are expressed relative to this value. (B) Effect of different SB 203580 concentrations on the activities of JNK1 and JNK2 in the presence of either 2 μM or 100 μM ATP. (C) COS-7 cells were transiently transfected with pPM7-RICK-KRdCst expression plasmid (1.3 μg/well) plus either control vector or pPM7 plasmids encoding RICK or RICK-T95M (0.2 μg/well). After metabolic labelling with [³²P]-orthophosphate and precipitation using StrepTactin beads, radioactively labelled RICK-KRdC was detected by autoradiography (panels 1, 3, and 5) prior to immunodetection with StrepTactin-HRP (panels 2, 4, and 6). (D) Amino acid residues surrounding Thr-106 in p38 were aligned with the corresponding sequences of various protein kinases binding to the PI 51 matrix. With the exception of CK1δ, all kinases sharing a bulky amino acid residue at the indicated position (grey background) show significantly higher IC₅₀ values than those having a threonine residue at this position.

FIG. 4. Generation of a functional Kinator II matrix. (A) Chemical structure of the BCR-ABL kinase inhibitor PD180970 compared with compound B in its free and immobilized form.

FIG. 5. In vitro association of total lysates from COS-7 cells expressing FGFR1 (A) or HeLa cells (B) with either control matrix or Kinator II matrix. Free compound B was present in the lysate where indicated. Both non bound proteins from the supernatants and bound proteins eluted from the matrix were immunoblotted with specific commercially available antibodies for FGFR, Src and Abl.

FIG. 6. HeLa cell lysates were subjected to affinity chromatography on a Kinator II column and different elution fractions were subjected to two-dimensional IEF/SDS-PAGE. Consecutive elution steps were performed with 5 mM ATP/20 mM MgCl₂ (A), 1 mM compound B (B), both 5 mM ATP/20 mM MgCl₂ and 1 mM compound B (C) and 7M urea/2M thiourea (D). The initial flow-through was dialyzed against running buffer and then reapplied onto the Kinator II column. Bound proteins were first eluted with 5 mM ATP/20 mM MgCl₂ (E) and subsequently eluted with 7M ureal 2M thiourea (F). Positions if protein kinase identified by mass spectrometry are indicated on the gels.

FIG. 7. Results from mass spectrometry analysis were confirmed by in vitro association of total lysates from HeLa cells or COS-7 cells expressing truncated GAK(24-646) protein fused to a C-terminal vesicular stomatitis virus G protein (VSV-G) epitope with either control matrix or Kinator II matrix. Free compound B was present in the lysate where indicated. Both non bound proteins from the supernatants and bound proteins eluted from the matrix were immunoblotted with specific commercially available antibodies for Weel, Rsk1, Yes, GSK3α/β, JNK, RICK, CK1α, and for the VSV-tag of GAK(24-646)-VSV-G (A) or Ack, NEK2, MEK2, Aurora A, p38, CSK, and AMPK (B).

FIG. 8. The concentration-dependent inhibition of p38a, RICK and GAK by compound B was determined. Kinase activities in the absence of inhibitor were set to 100% and remaining activities at different compound B concentrations are expressed relative to this value.

FIG. 9. Cellular inhibition of p38 and RICK by compound B. (A) HeLa cells were pretreated with the indicated concentrations of compound B for 15 min prior to either a 30 min stimulation with 10 μg/ml anisomycin or a 5 min stimulation with 50 ng/ml EGF. After gel electrophoresis, samples from anisomycin-treated cells were immunoblotted with antibody specific for phosphorylated MAPKAP kinase-2 (upper panel) or antibody specific for phosphorylated Rsk1 (lower panel). (B) COS-7 cells were transiently transfected with either control plasmid, pPM7-RICK-KRdCst expression plasmid or cotransfected with pPM7 plasmids encoding RICK and RICK-KRdCst After metabolic labelling with [³²P]-orthophosphate and precipitation using StrepTactin beads, radioactively labelled RICK-KRdC was detected by autoradiography (upper panel) prior to immunodetection with StrepTactin-HRP (lower panel).

FIG. 10. Activation-dependent protein kinase binding to Kinator III. (A) Total cell lysates from HuH-7 cells were incubated with either control beads or Kinator III beads in the absence or presence of PKC-specific cofactors (100 μg/ml phosphatidylserine, 20 μg/ml diacylglycerol and 0.45 mM CaCl₂). Both non bound proteins from the supernatants and bound proteins eluted from the matrix were immunoblotted with specific commercially available antibodies for PKCα. (B) Total cell lysates from HeLa cells stimulated for the indicated times with 100 ng/ml EGF before lysis and in-vitro association with Kinator III beads. Upon gel electrophosresis, immunoblotting of bound proteins was performed with antibodies specific for Rsk1 (upper panel). In parallel, total cell lysates were immunoblotted with the same antibody (lower panel).

FIG. 11. HeLa cell proteins purified by affinity chromatography on the Kinator III matrix were separated by 16-BAC/SDS-PAGE and stained with Coomassie blue. The indicated spots (arrows) were analysed by mass spectrometry. Spots containing identified protein kinases, quinone reductase 2 (HQO2), adenosine kinase and pyruvate kinase are marked by their names.

FIG. 12. In vitro characterization of identified proteins. (A) The concentration-dependent inhibition of full length SLK by compound C was determined. Kinase activity in the absence of inhibitor was set to 100% and remaining activities at different compound C concentrations are expressed relative to this value. (B) Lysates from COS-7 cells transfected with myc-tagged adenosine kinase were incubated with either control beads or Kinator III beads prior to immunoblotting with myc-epitope specific antibody. (C) Competitive in vitro inhibition of human quinone reductase NQO2 by compound C with a Kᵢ value of 16.5 μM.

FIG. 13. HeLa cell lysates were subjected to in vitro association with control, Kinator III, Kinator IV or Kinator V beads. Both non bound proteins from the supernatants and bound proteins eluted from the different matrices were resolved by gel electrophoresis and then analysed in parallel by either silverstaining or immunoblotting with commercially available antibodies specific for Rsk1. PKCα, PKCε, GSK3α/β, and Cdk2.

FIG. 14. The concentration-dependent inhibition of Cdk2 by compounds C, D and E was determined. Kinase activity in the absence of inhibitors was set to 100% and remaining activities at different compound C, D and E concentrations are expressed relative to this value.

FIG. 15. HeLa cell lysates were subjected to in vitro association with control, Kinator III, Kinator IV or Kinator V beads followed by gel electrophoresis and immunoblotting with EGFR-specific antibodies.

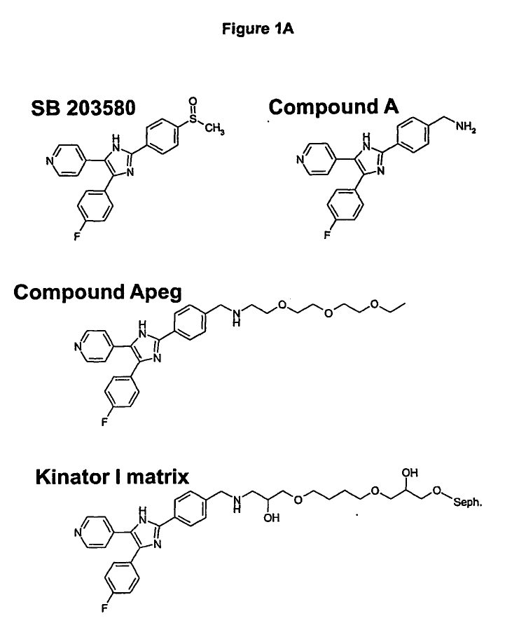

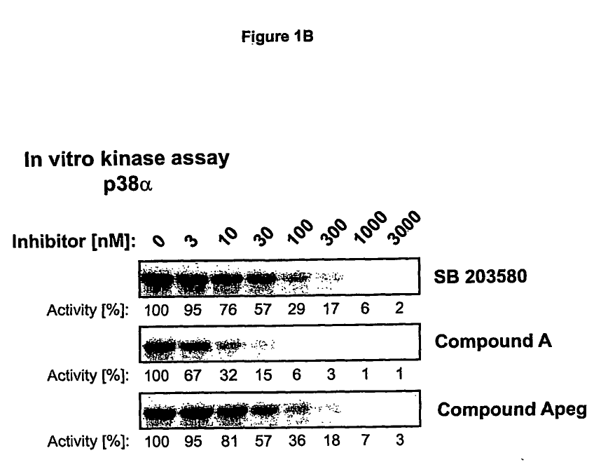

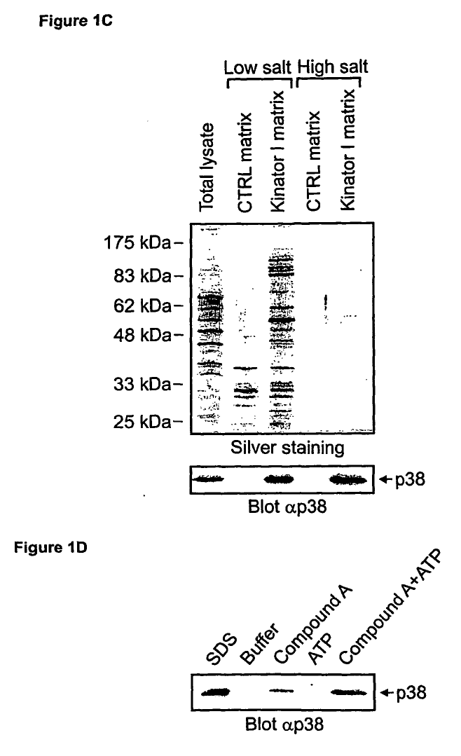

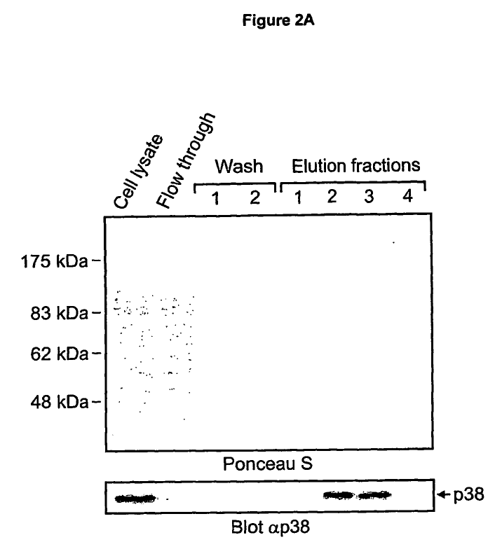

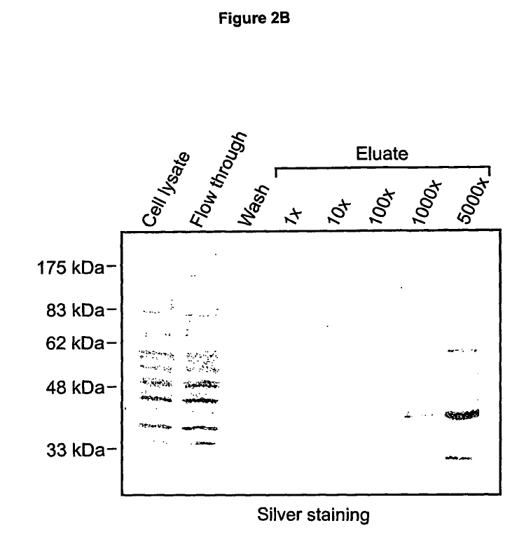

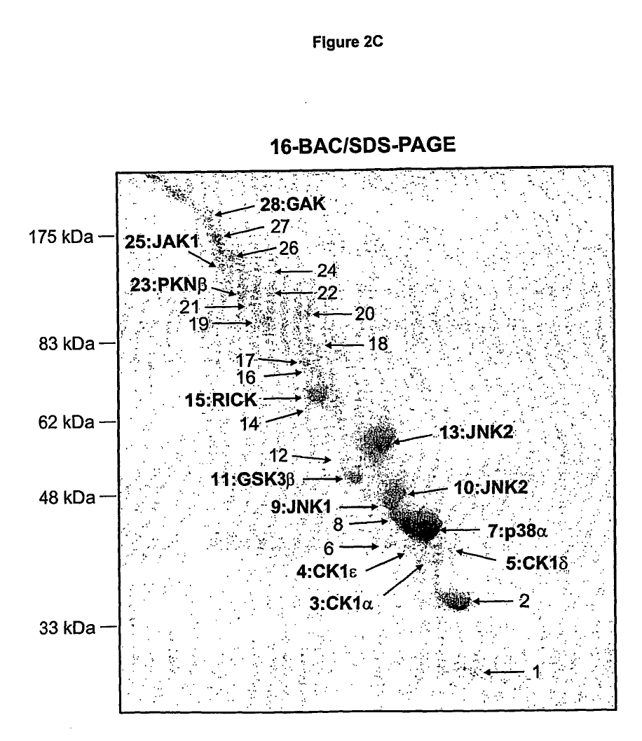

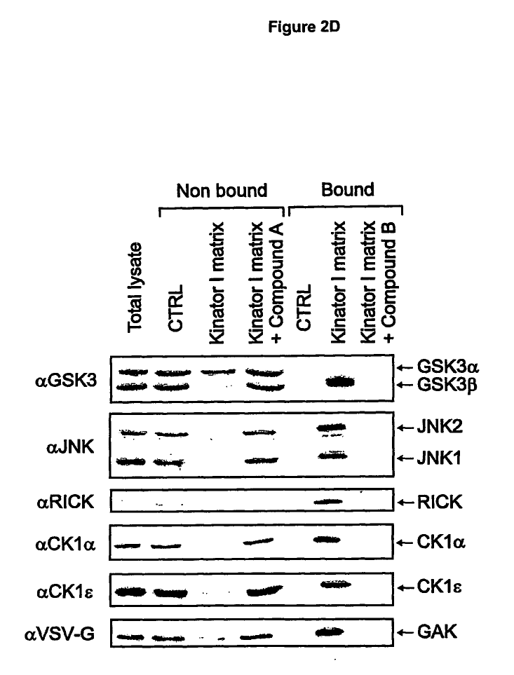

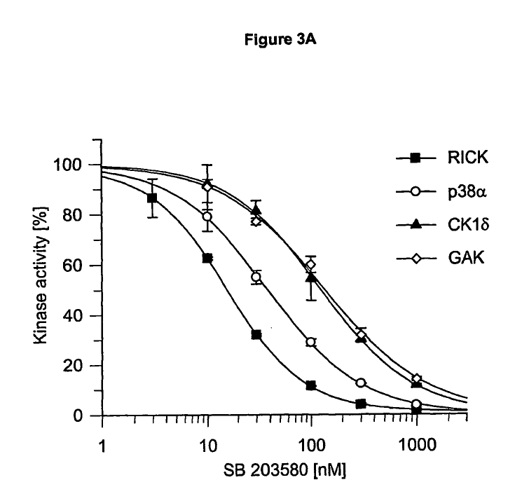

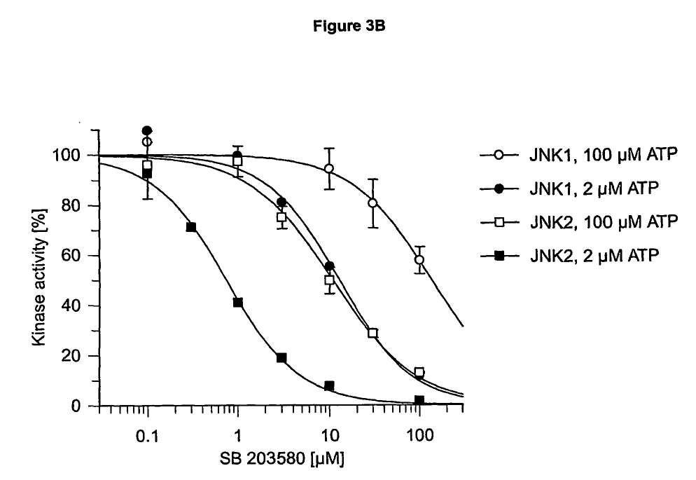

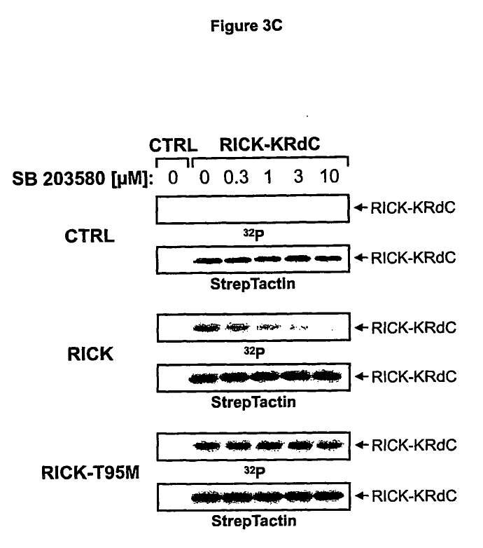

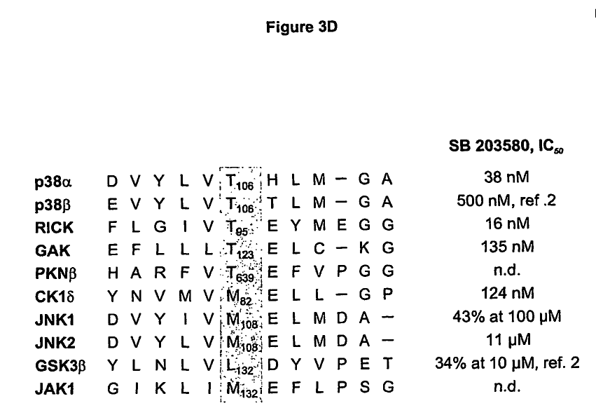

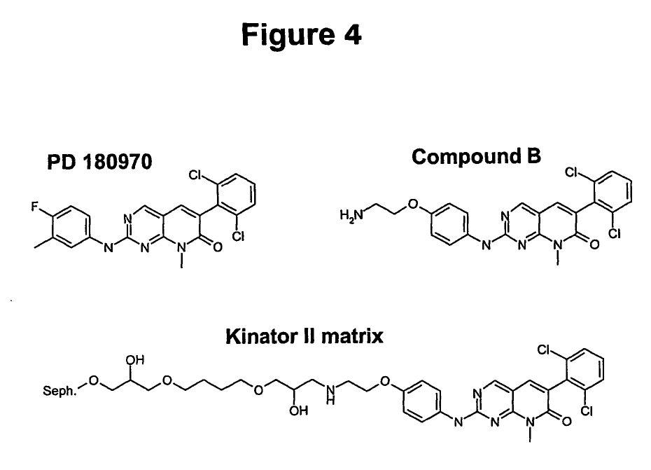

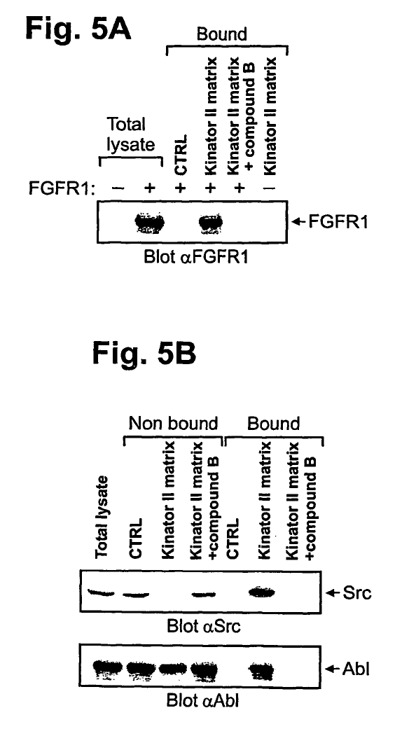

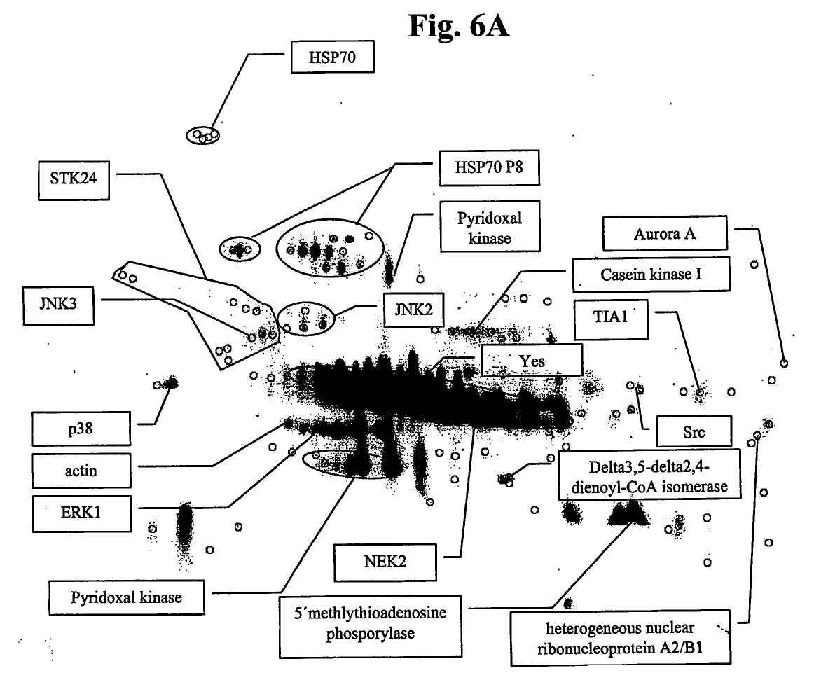

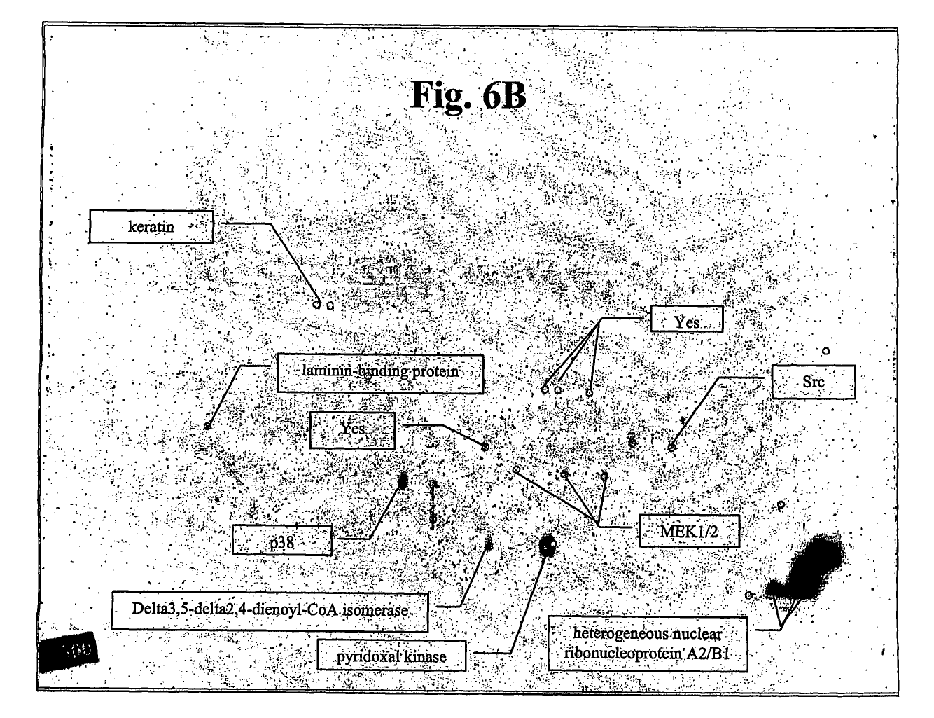

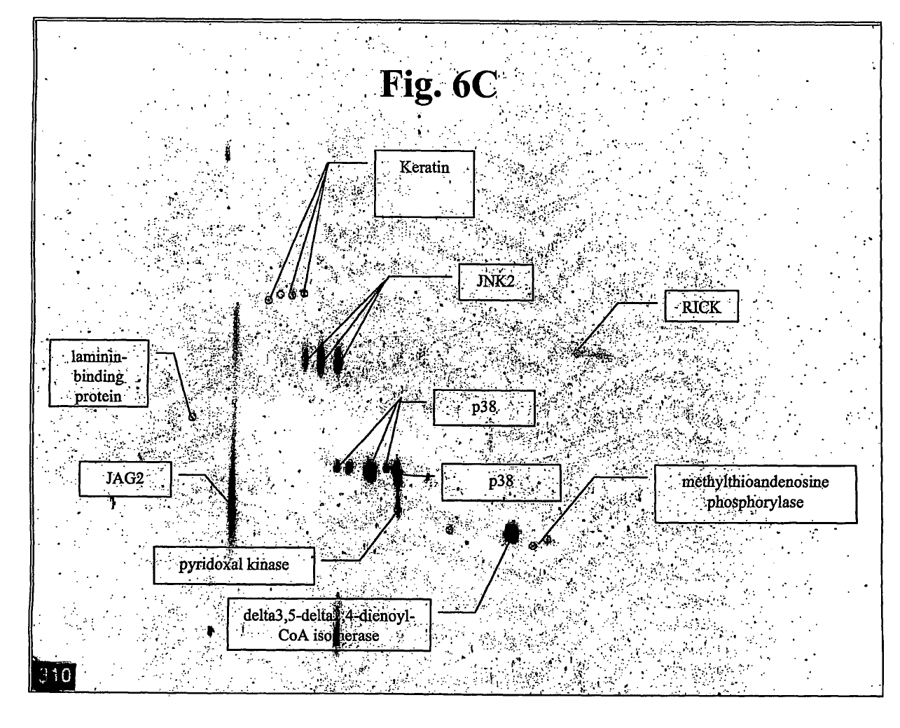

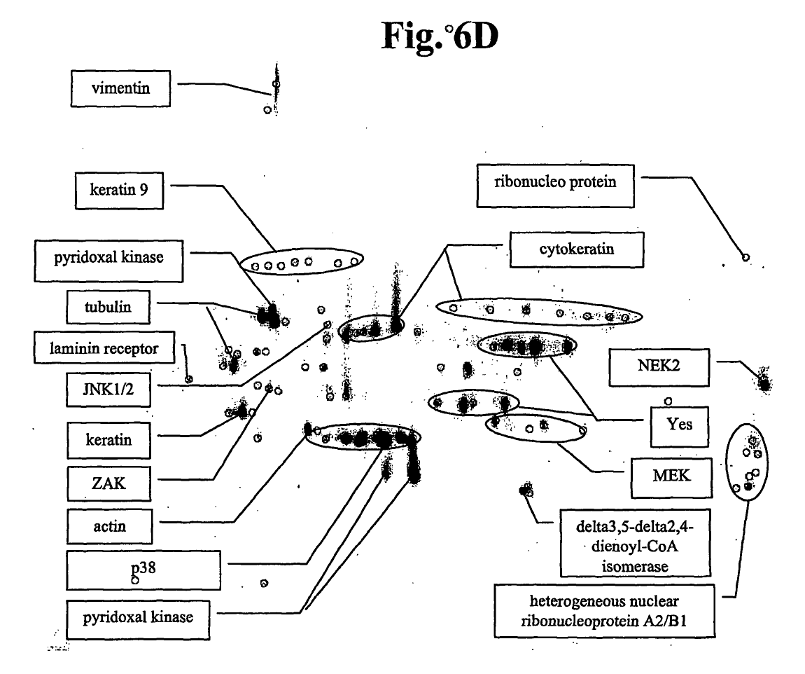

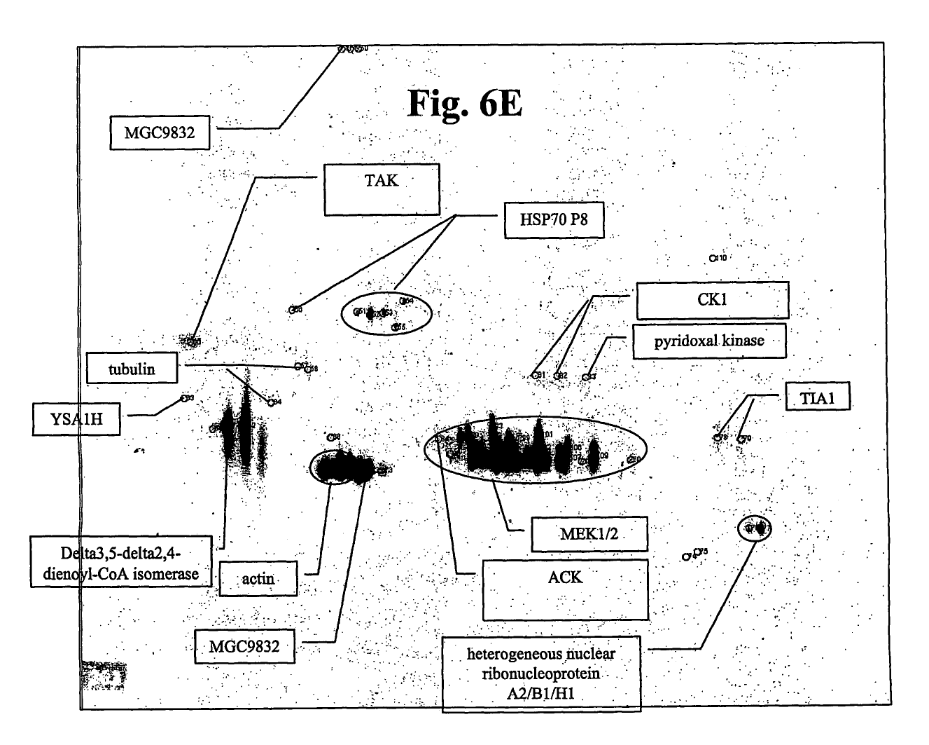

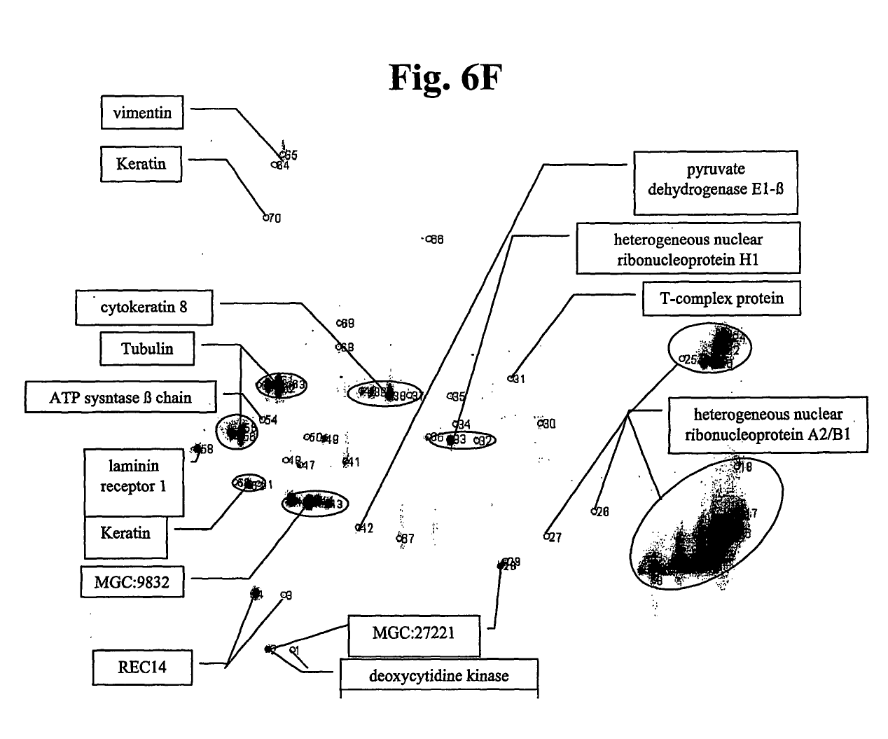

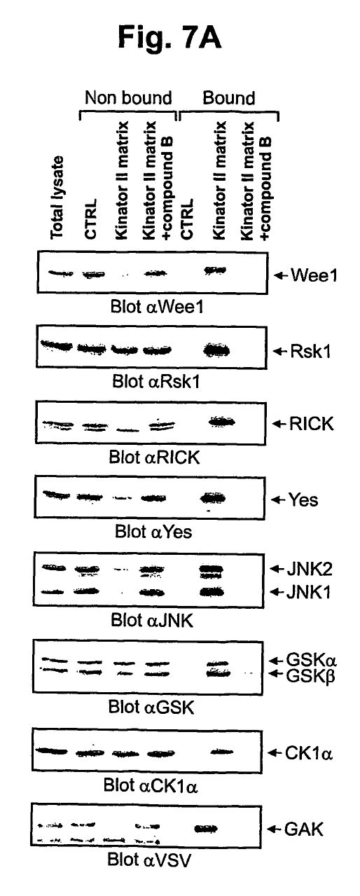

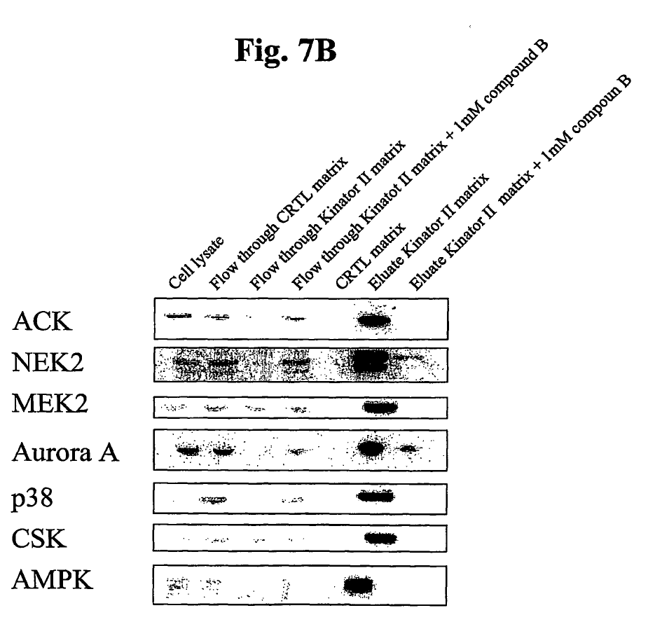

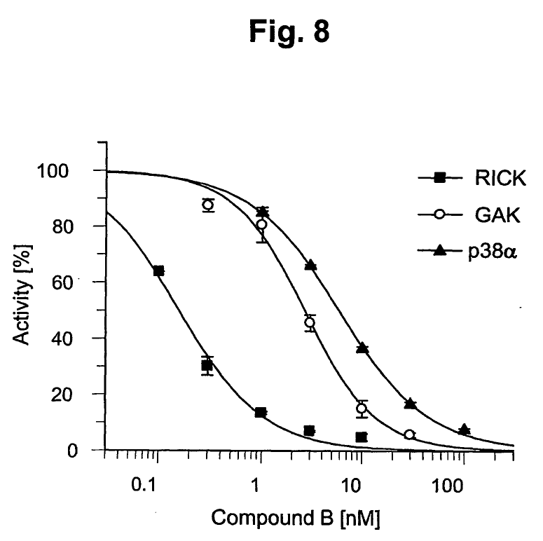

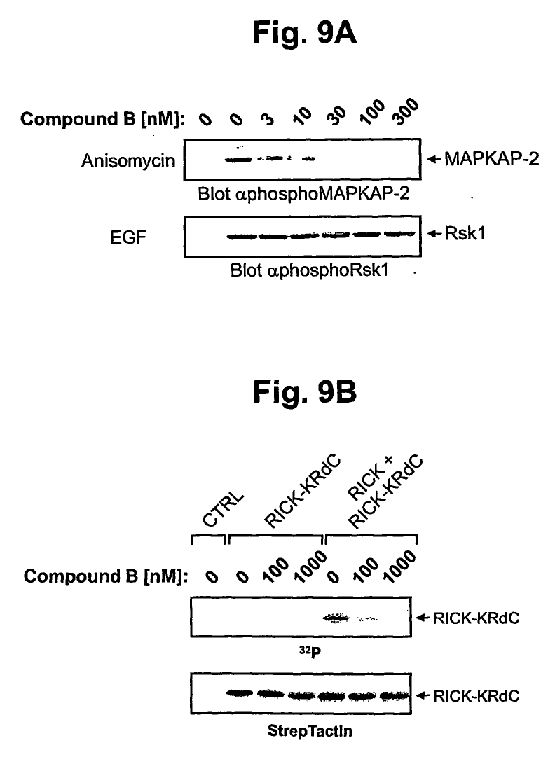

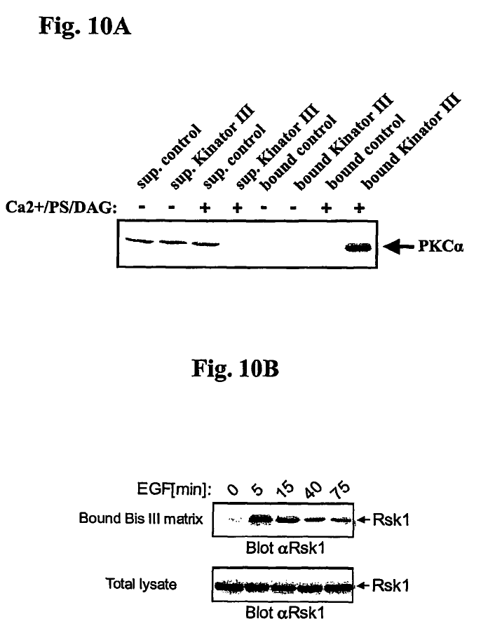

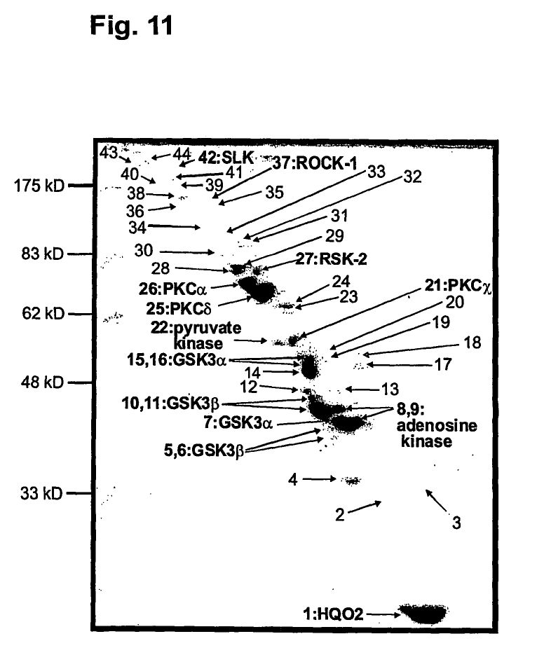

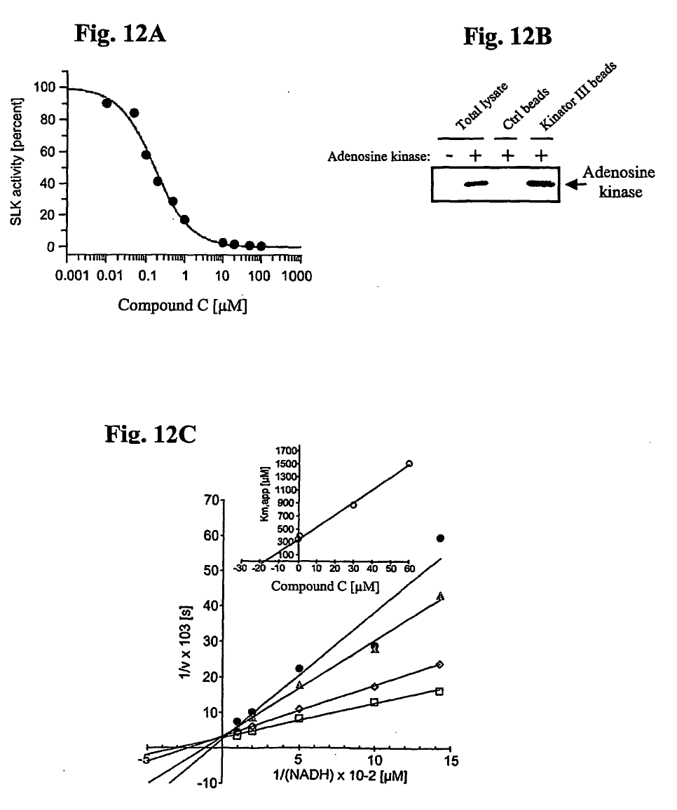

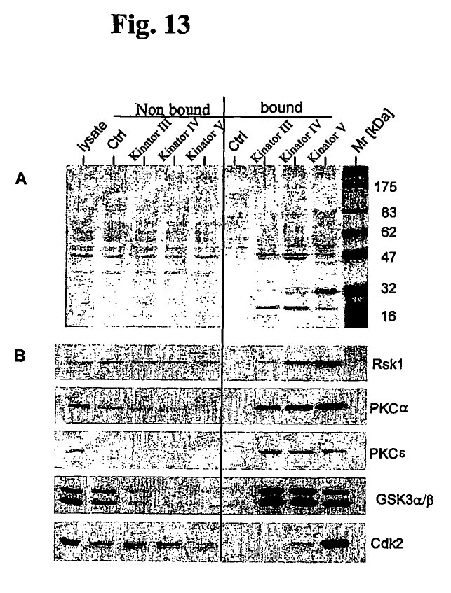

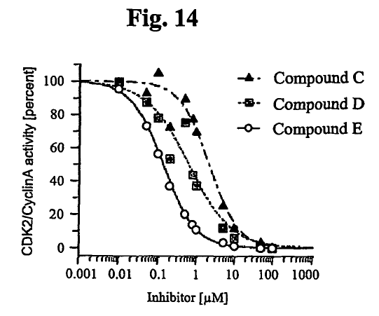

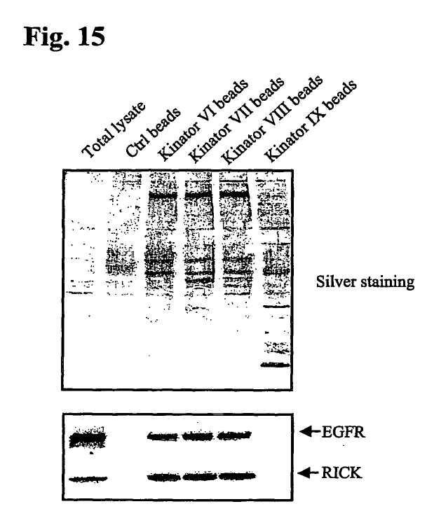

Based on analysis of kinase-inhibitor structures we have further generated a set of novel protein kinase inhibitor derivatives, which retain the ability to interact with their cellular targets upon covalent immobilization on solid support material. These structures belong to different classes of compounds. For each scaffold, several examples are given with linkers for covalent immobilization:

Quinoxalines:

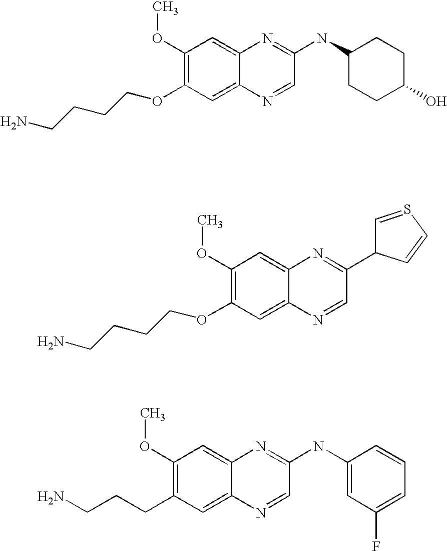

Quinolines:

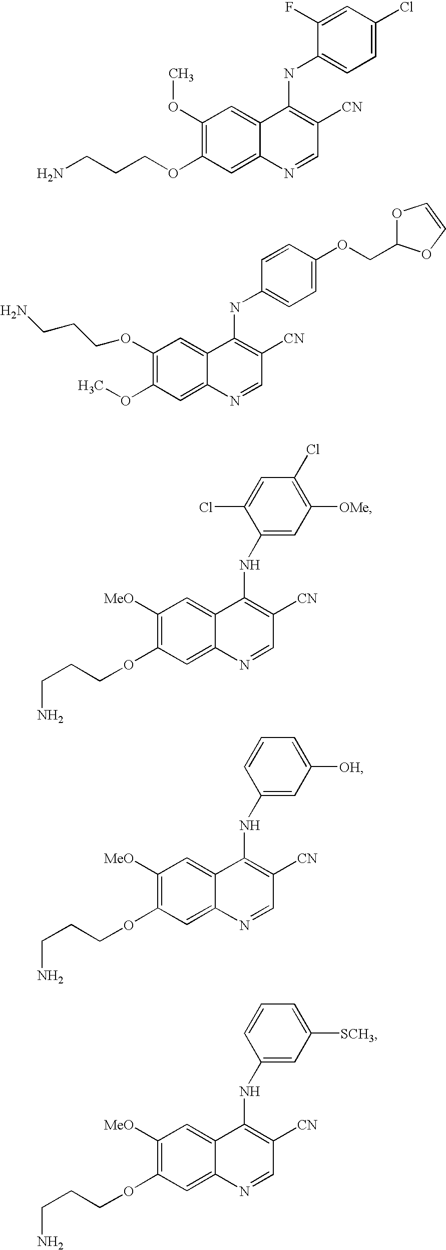

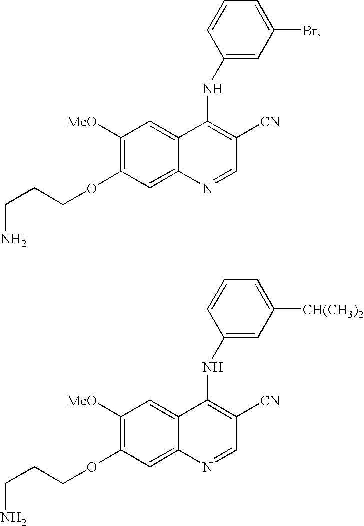

Quinazolines:

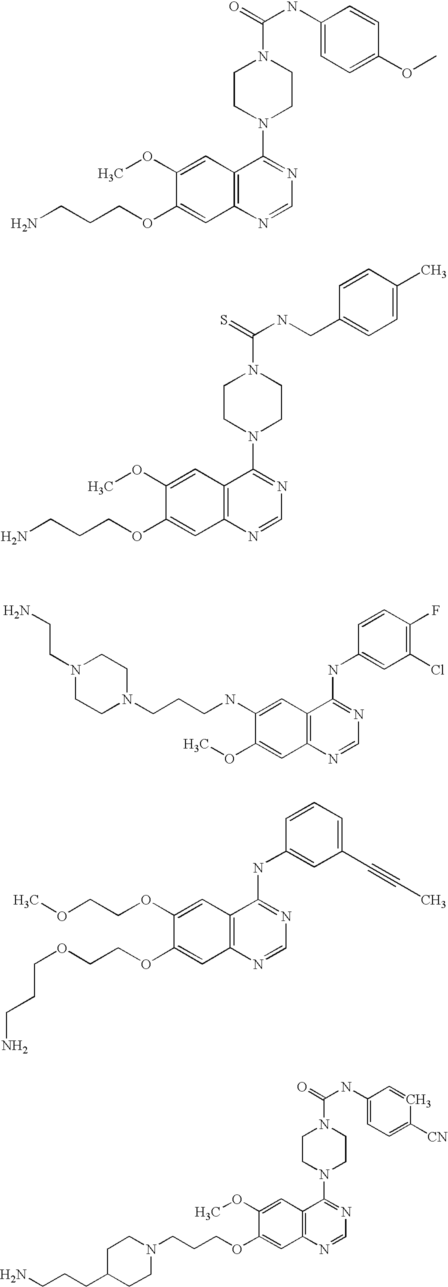

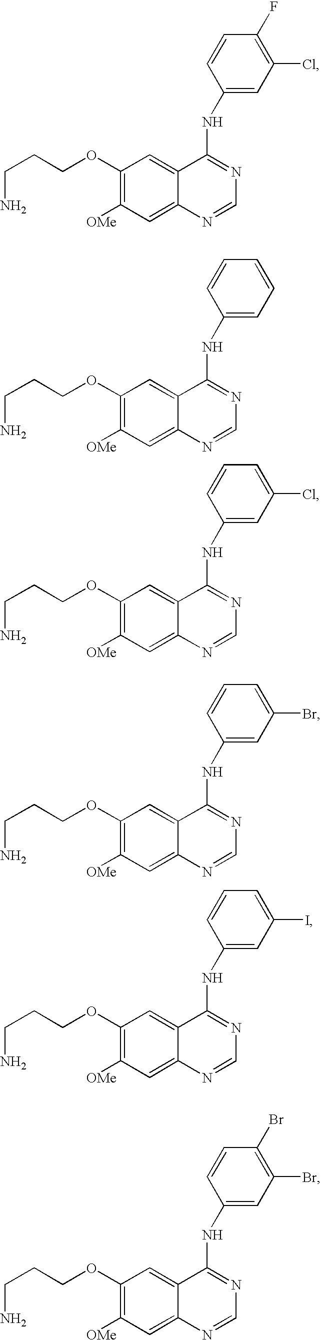

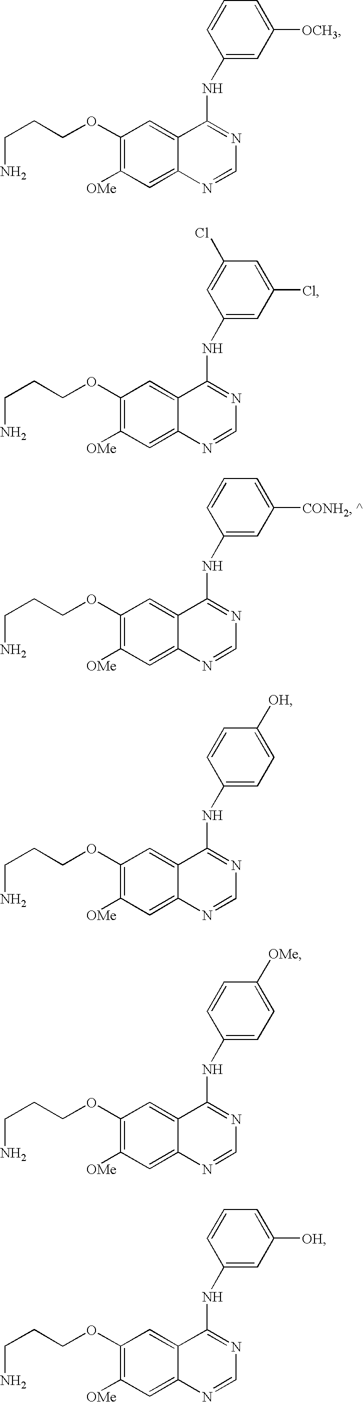

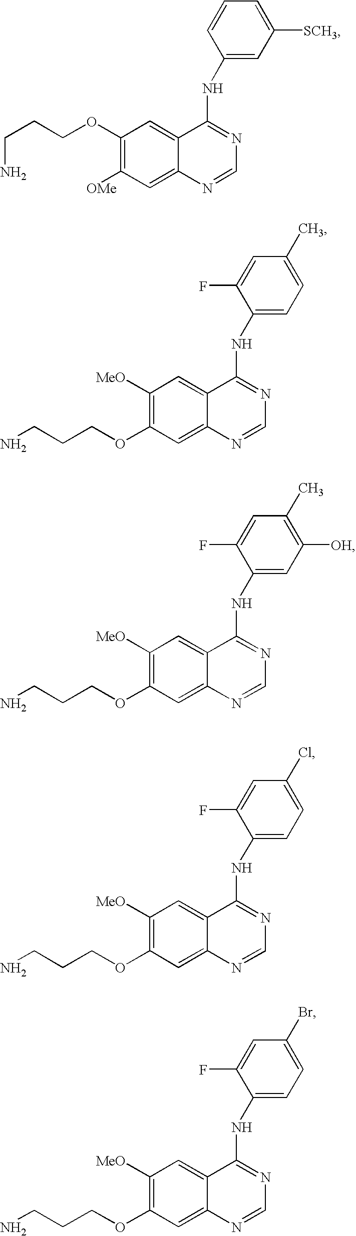

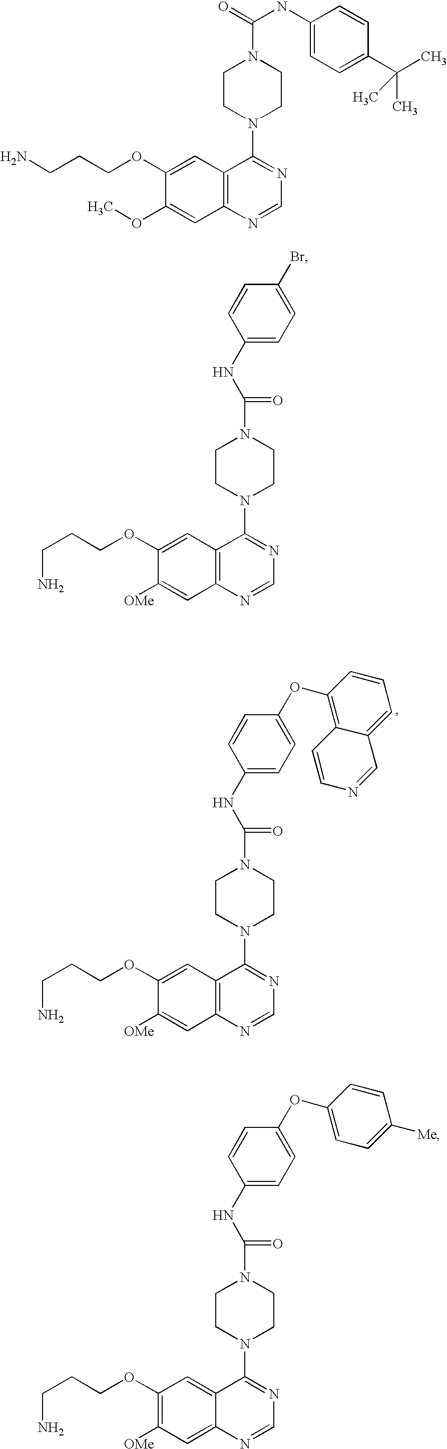

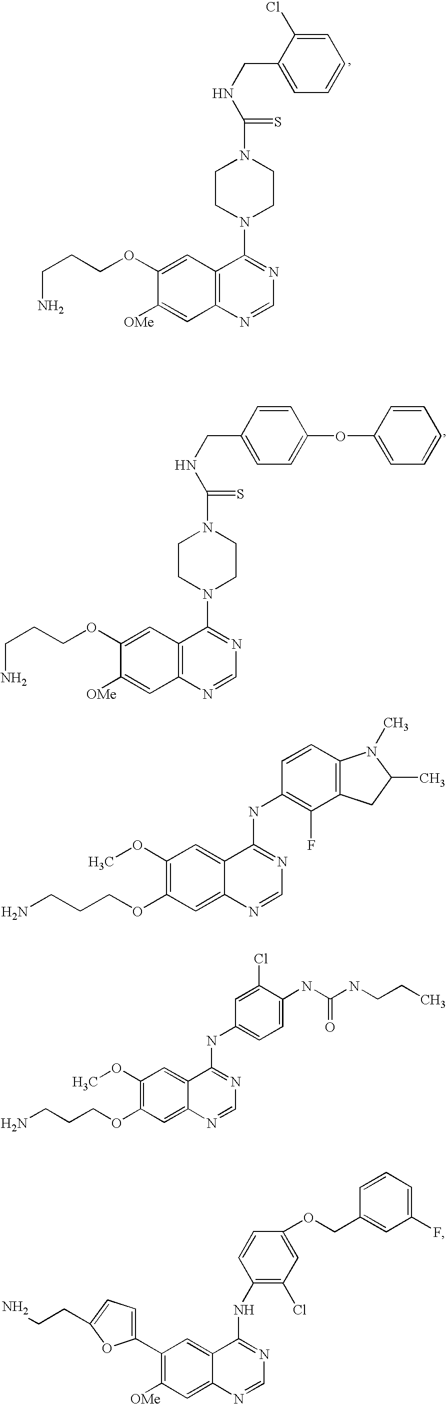

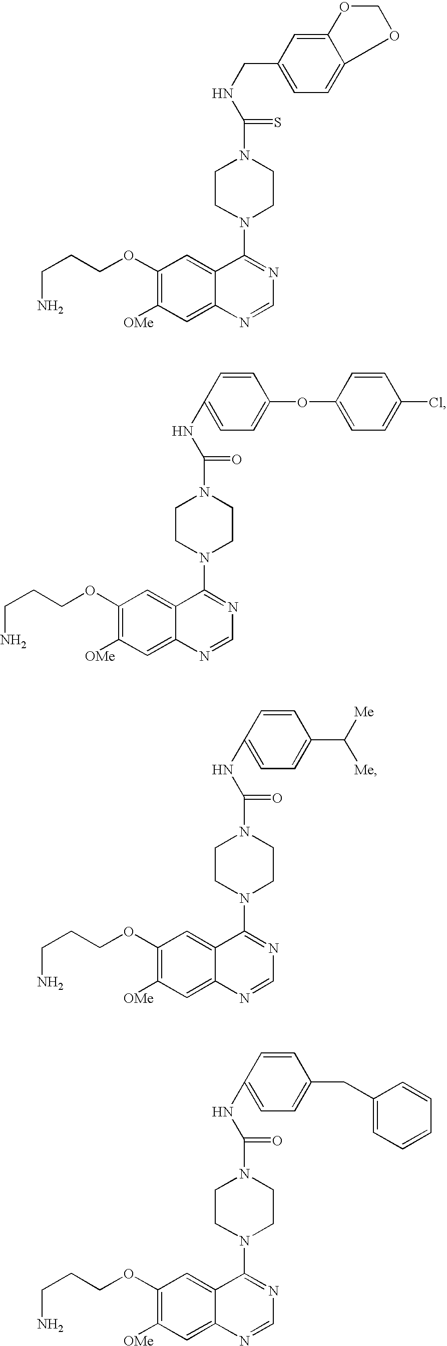

Pyrrolopyrazines:

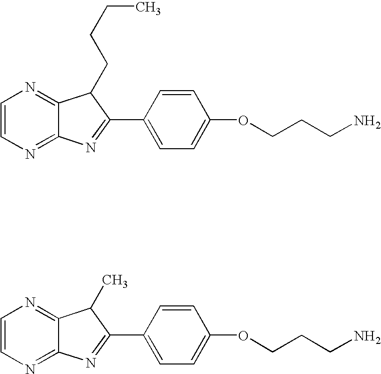

Pyridopyrimidines:

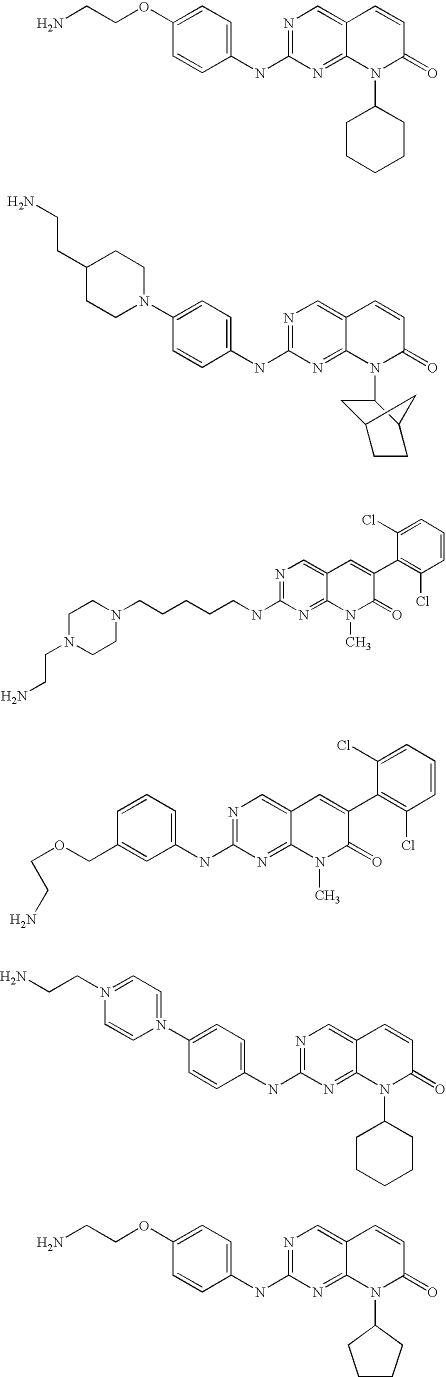

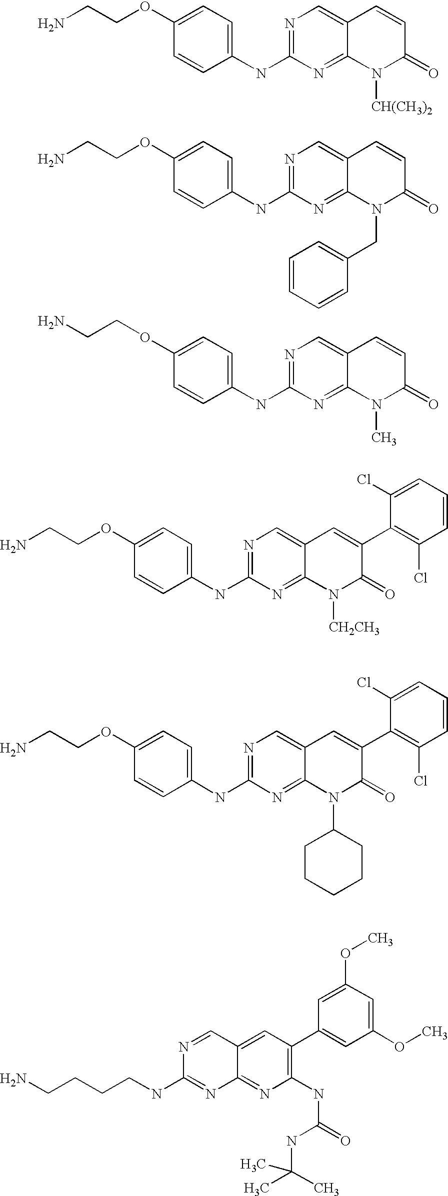

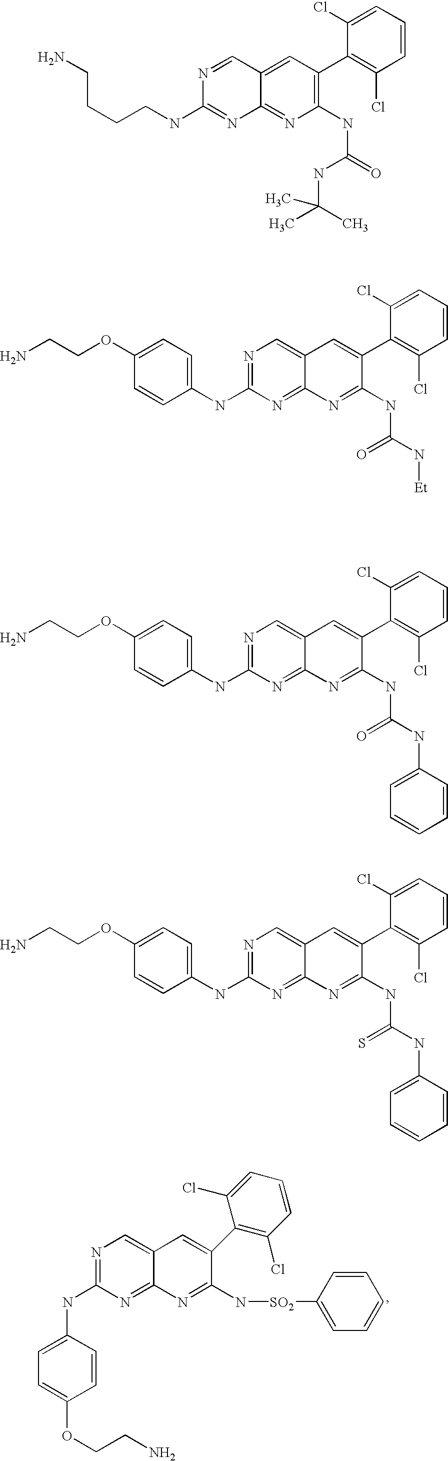

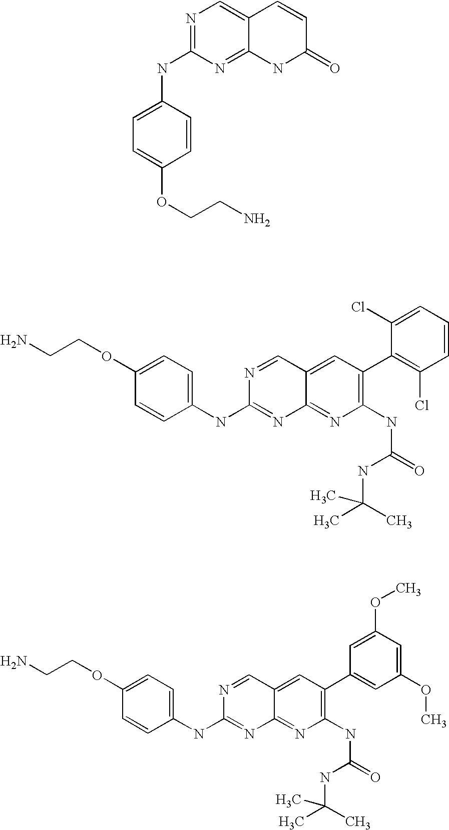

Pyridinylimidazoles:

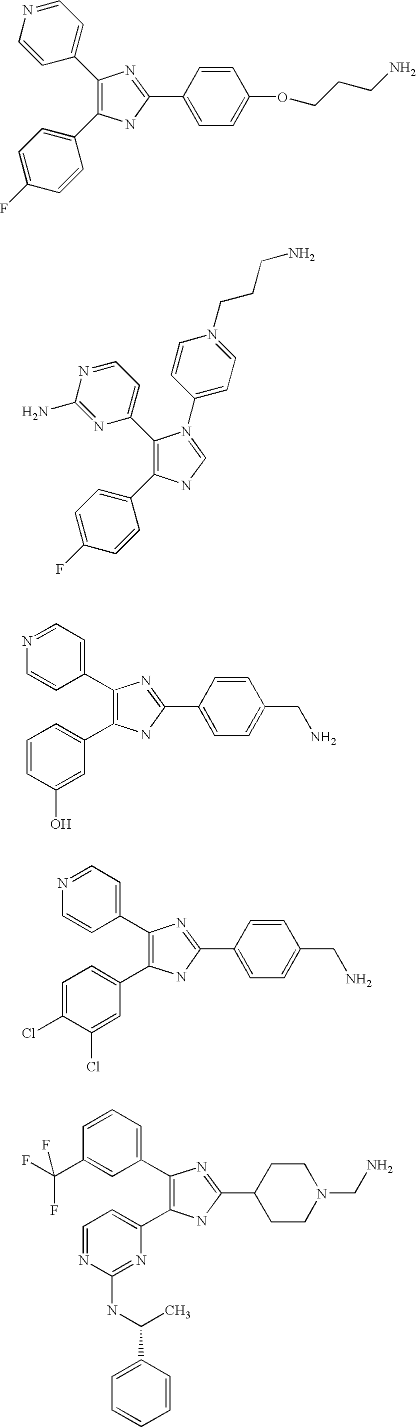

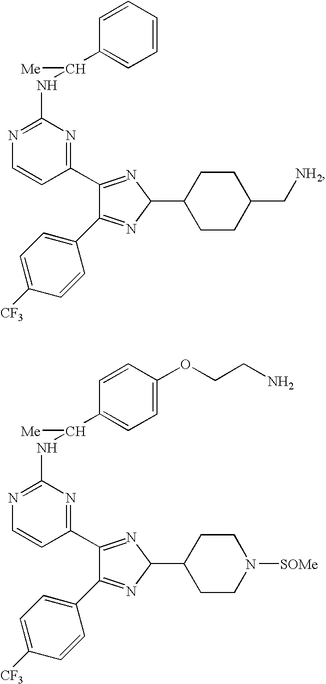

Pyrazolo pyrimidines:

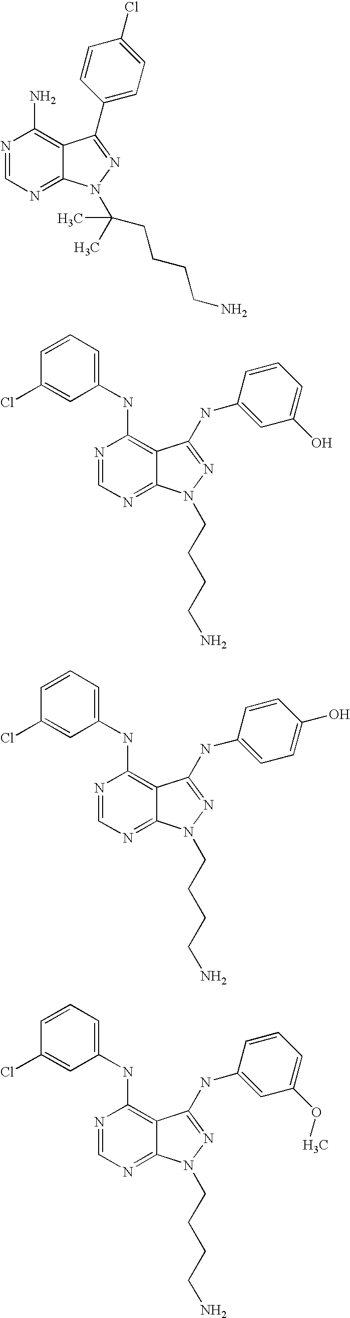

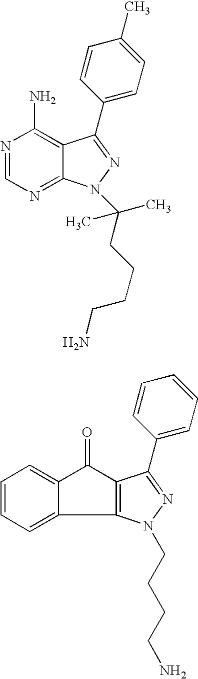

Indolinones:

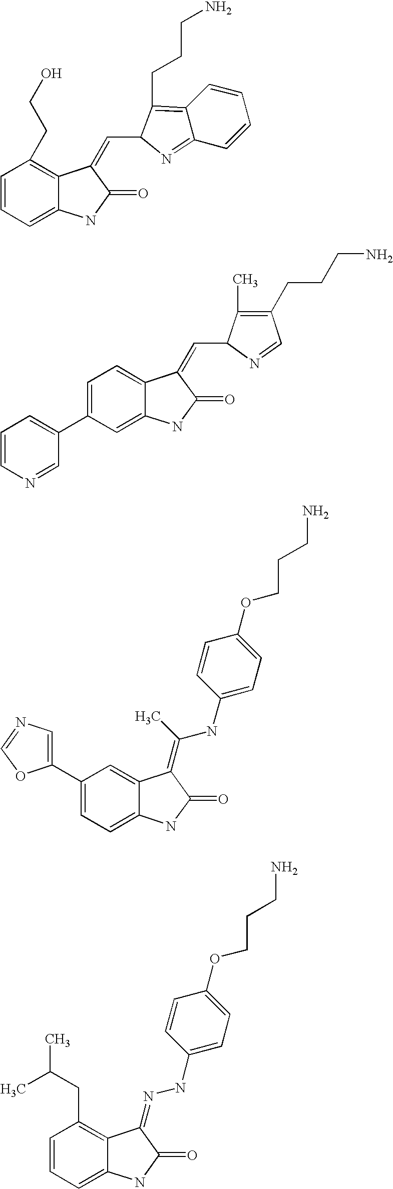

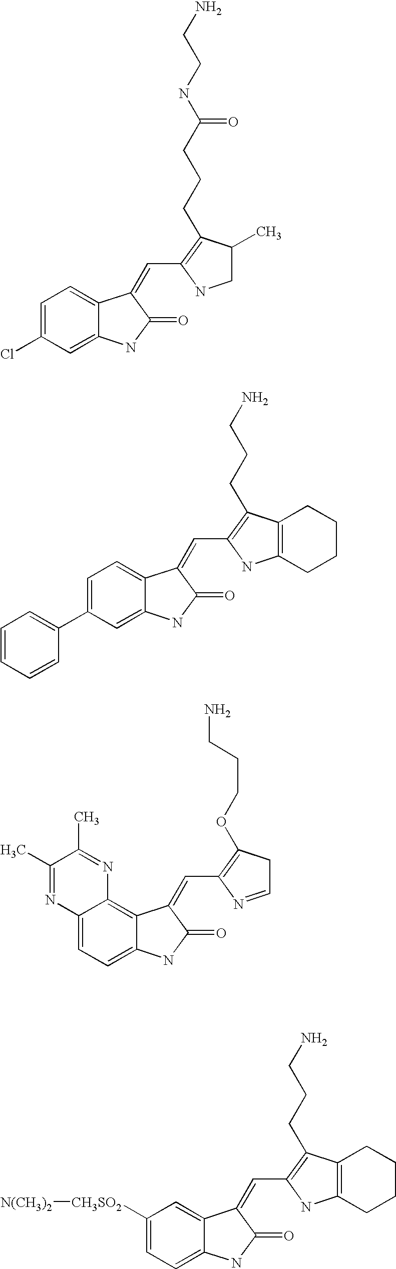

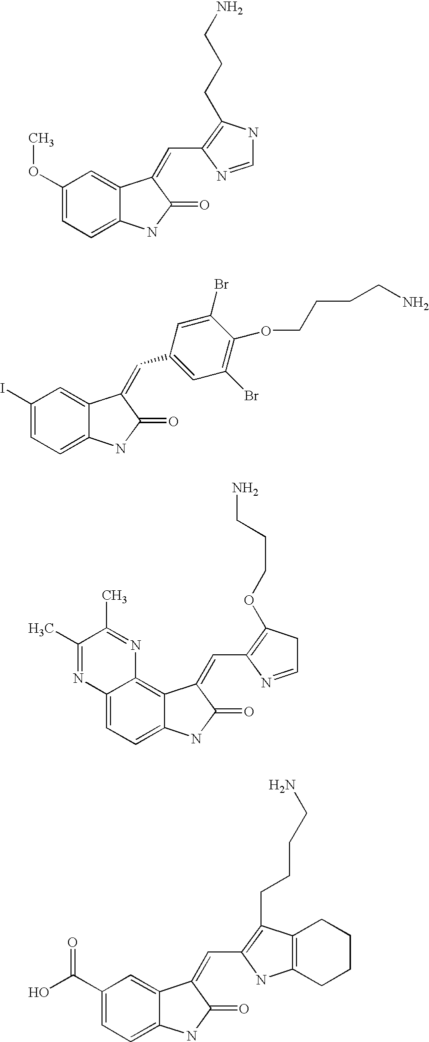

Aryl-bisaryl urea compounds:

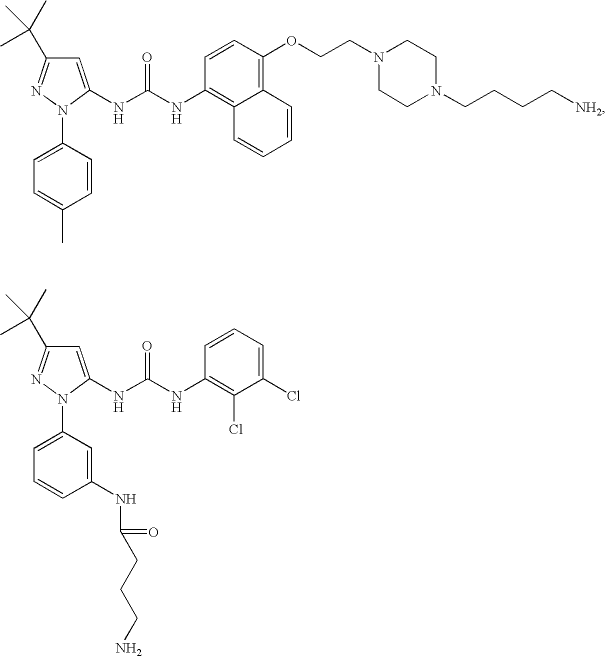

Phenylaminopyrimidines:

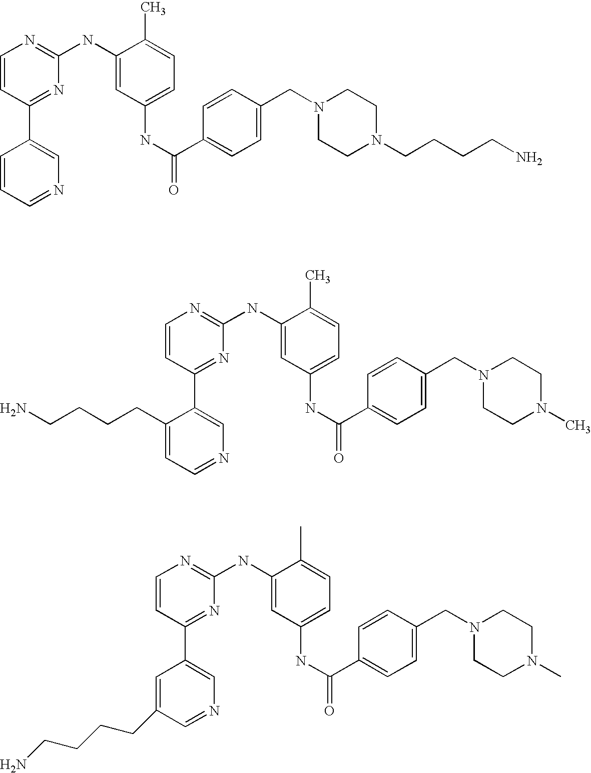

1-50. (canceled)

51. A medium for separating at least one ATP binding protein from a pool of proteins, the medium comprising at least one compound of the general formula I 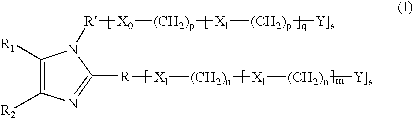
formulas IIa and IIb (compound class B) 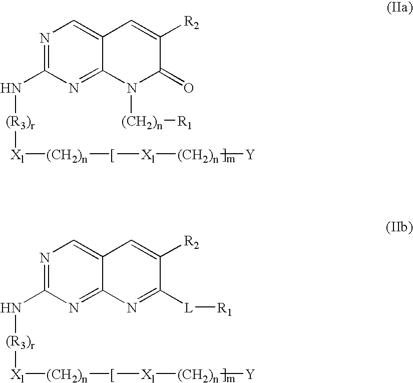
wherein
each L is independently selected from —NH—CO—NH—, —NH—SO₂—, or —NH—CS—NH—,
each X is independently selected from —CH₂—, —NH—, —O—, —S—, 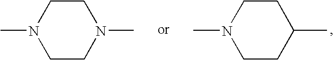
each Y is independently selected from —NH₂, —NHR₁, —OH, —SH or —SO(CH₃),
each l is independently selected to be 0 or 1,
each m is independently selected to be an integer from 0 to 10,
each n is independently selected to be an integer from 0 to 10,
each o is independently selected to be 0 or 1,
each p is independently selected to be an integer from 0 to 10,
each q is independently selected to be an integer from 0 to 10,
each r is independently selected to be an integer from 0 to 2,
R and R′ are independently of each other —H, 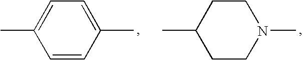
and each s is independently selected to be 0 or 1, with the proviso that s=0 if R or R′ is H, —each R₁ is independently selected from —H, C₁-C₆ alkyl (linear or branched), bicyclo[3.3.1]heptanyl, or an unsubstituted or partially or fully substituted C₃-C₈ cycloalkyl, aryl, pyridinyl or pyrimidinyl, substituted by —F, —Cl, —Br, —I, —CN, —OH, —SH, —NH₂, —NHCHR₂R₂, C₁-C₆-alkoxy, C₁-C₆-alkylthio, C₁-C₆-haloalkyloxy, partially or fully halogenated C₁-C₆ alkyl and/or —X₁—(CH₂)ₙ—Y (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
each R₂ is independently selected from —H, C₁-C₆ alkyl (linear or branched), an unsubstituted or partially or fully substituted aryl,substituted by —F, —Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), and
each R₃ is independently selected from X, an unsubstituted or partially or fully substituted aryl, pyridinyl or pyrimidinyl, substituted by —F, —Cl, —Br, —I, —CN, —OH, —SH, —NH₂, —NHCHR₁R₁, C₁-C₆-alkoxy, C₁-C₆-alkylthio, C₁-C₆-haloalkyloxy, and/or partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
formulas IIa and IIIb (compound class C) 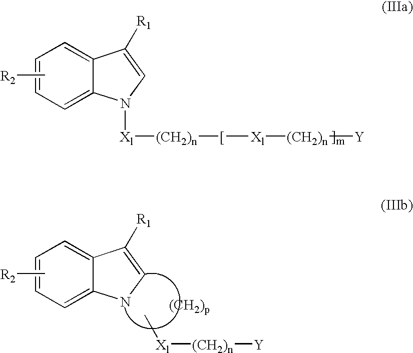
wherein
each R₁ is independently selected from 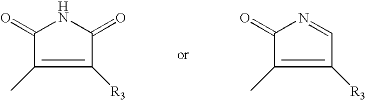
each R₃ is independently selected from -indolyl, N—(C₁-C₆ alkyl)-indolyl (alkyl is linear or branched), —NHR₁′, —S—R₁′, or —O—R₁′,
R₁′ is —H, C₁-C₆ alkyl (linear or branched) or aryl,
each R₂ is independently selected from —H, —F, —Cl, —Br, —I, —CN, —OH, —SH, —NH₂, C₁-C₆-alkyl (linear or branched), C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
each X is independently selected from —CH₂—, —NH—, —O—, —S—, 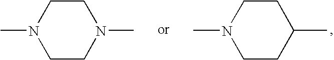
each Y is independently selected from —NH₂, —NHR₁, —OH, —SH or —SO(CH₃),
each l is independently selected to be 0 or 1,
m is an integer from 0 to 10,
each n is independently selected to be an integer from 0 to 10,
p is an integer from 2 to 6
formula IV (compound class D) 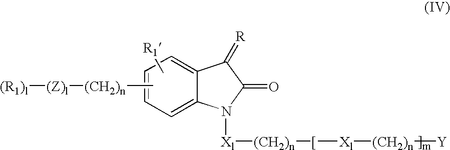
wherein
each X is independently selected from —CH₂—, —NH—, —O—, —S—, 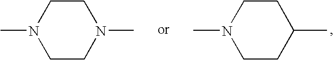
each Y is independently selected from —NH₂, —NHR₁, —OH, —SH or —SO(CH₃),
Z is —SO₂—NR₁R₁, —CO, —O—CO—, —NH—CO—, —COO—, —CO—NH—, —OCH₂—, —SCH₂—,
each l is independently selected to be 0 or 1,
m is an integer from 0 to 10,
each n is independently selected to be an integer from 0 to 10,
R is ═CR₁L, ═N—NH-L,
each R₁ is independently selected from —H, C₁-C₆ alkyl (linear or branched), unsubstituted or partially or fully substituted aryl, pyridinyl, pyrimidinyl, C₃-C₈ cycloalkyl substituted by —F, —Cl, —Br, —I, —CN, —OH, —SH, —NH₂, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group, C₁-C₆-alkylthio denotes an S-alkyl group, C₁-C₆-haloalkyloxy denotes an halogen-alkyl-O group, C₁-C₆-haloalkyl denotes an halogen-alkyl group wherein the alkyl group is linear or branched), —F, —Cl, —Br, —I, —COOH, —(CH₂)ₙ—OH, oxazolyl, thiazolyl, thienyl, pyrrolyl, furanyl, imidazolyl, pyrazolyl, pyridinyl, pyrimidinyl, R₁′ is independently selected from H or R₁ and R₁′ may form together the following substituted ringsystem 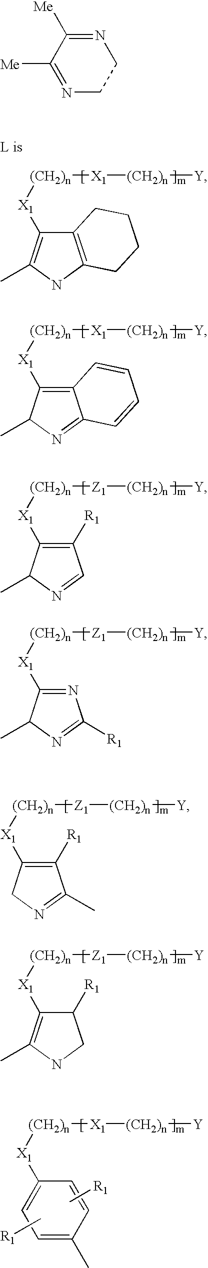
formulas V and VI (compound class E and F) 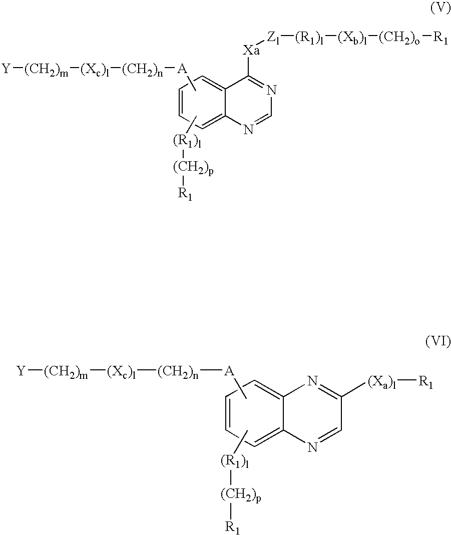
wherein
A, Xₐ, X_{b}, and X_{c}, are independently selected to be Z, —CH₂—, —NH—, —O—, —S—, 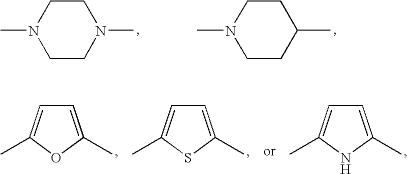
each Y is independently —NH₂, —NHR₁, —OH, —SH or —SO(CH₃),
each Z is independently selected from —SO₂—NR₁—, —CO—, —O—CO—, —NH—CO—, —COO—, —CO—NH—, —CS—NH—, —OCH₂—, —SCH₂—, or —NH—CO—NH—,
each l is independently selected to be 0 or 1,
each m is independently selected to be an integer from 0 to 10,
each n is independently selected to be an integer from 0 to 10,
each o is independently selected to be an integer from 0 to 10,
each p is independently selected to be an integer from 0 to 10,
each R₁ is independently selected from —H, —O—, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or partially or fully substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), 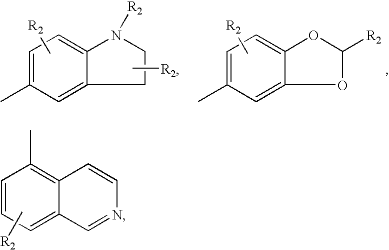
each R₂ is independently selected from —F, —Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
formula VII (compound class G) 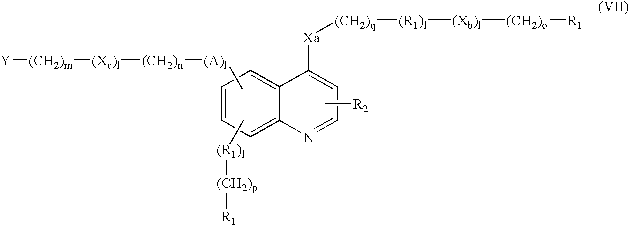
formula VIII (compound class H) 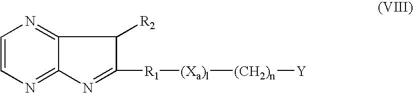
formula IX (compound class I) 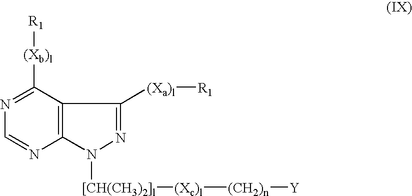
formula X (compound class J) 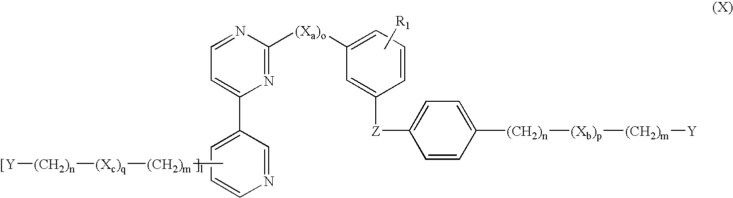
or formula XI (compound class K) 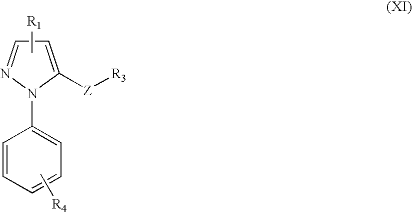
wherein
A, Xₐ, X_{b}, and X_{c} are independently selected from Z, —CH₂—, —NH—, —O—, —S—, 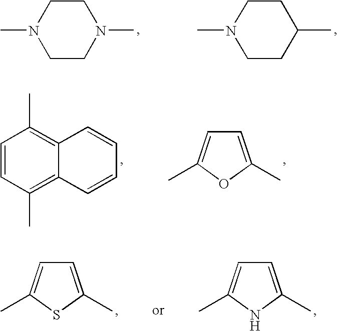
each Y is independently selected from —H, —NH₂, —NHR₁, —OH, —SH or —SO(CH₃),
each Z is independently selected from —SO₂—NR₁—, —CO—, —O—CO—, —NH—CO—, —COO—, —CO—NH—, —OCH₂—, —SCH₂—, —NH—CO—NH—,
each l is independently selected to be 0 or 1,
each m is independently selected to be an integer from 0 to 10,
each n is an integer independently selected from 0 to 10,
each of o, p, q is an integer independently selected from 0 to 10,
each R₁ is independently selected from —H, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), —F, —Cl, —Br, —I, —COOH, —NH₂, 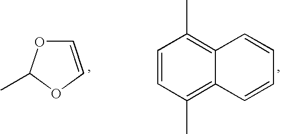
each R₂ is independently selected from —F, —Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
R₃ is —H or —(R₁)₁—(Xₐ)₁—(CH₂)ₙ—(X_{b})₁—(CH₂)ₙ—(Y)₁R₁,
R₄ is —H or —(R₁)₁—(Z)₁—(CH₂)ₙ—(X_{b})₁—(CH₂)ₙ—(Y)₁—R₁
covalently immobilized on a support material via the group Y.

52. The medium according to claim 51, wherein at least one of the compounds 4-[4-(4-fluoro-phenyl)-5-pyridine-4-yl-1H-imidazole-2-yl]-benzylamine, 2-[4-(2-Amino-ethoxy)-phenylamino]-6-(2,6-dichloro-phenyl)-8-methyl-8H-pyrido[2,3-d]pyrimidine-7-one, 2-[1-(3-aminopropyl)-1H-indole-3-yl)maleimide, 3-[1-(3-Aminopropyl)-1H-indol-3-yl]-3-(1H-indol-3-yl)-maleimide , 3-[1-(3-Aminopropyl)-1H-indol-3-yl]-4-(1-methyl-1H-indol-3-yl)maleimide, 3-(8-Aminomethyl-6,7,8,9-tetrahydropyrido-[1,2-a]-indol-10-yl)-4-(1-methyl-1H-indol-3-yl)-maleimide, [6-(3-Amino-propoxy)-7-methoxy-quinazolin-4-yl]-(3-chloro-phenyl)-amine, 6-(3-Amino-propoxy)-7-methoxy-quinazolin-4-yl]-(3-chloro-4-fluoro-phenyl)-amine, 6-(3-Amino-propoxy)-7-methoxy-quinazolin-4-yl]-(3-bromo-phenyl)-amine and 4-[4-(4-Amino-butyl)-piperazin-1-yl-methyl]-N-[4-methyl-3-(4-pyridin-3-yl-pyrimidin-2-ylamino)-phenyl]-benzamide is immobilized on the support material.

53. The medium according to claim 51, wherein the support material comprises agarose and/or modified agarose material.

54. The medium according to claim 51, wherein the support material comprises ferro- or ferrimagnetic particles.

55. The medium according to claim 54, wherein the ferro- or ferrimagnetic particles have a surface area of about 190 m²/g or greater, determined according the BET method and/or wherein the average size of the ferro- or ferrimagnetic particles is from 5 to 25 μm in diameter.

56. The medium according to claim 54, wherein the ferro- or ferrimagnetic particles comprise about 30 to 50% by weight of Fe₃O₄ and about 50 to 70% by weight of SiO₂.

57. The medium according to claim 51, wherein the pool of proteins is a proteome, a cell lysate or a tissue lysate.

58. The medium according to claim 51, wherein the ATP binding protein is a protein kinase.

59. A method for enriching, purifying or depleting at least one ATP binding protein from a pool of proteins containing at least one ATP binding protein, the method comprising the following steps:
a) immobilizing at least one compound of the general formula I 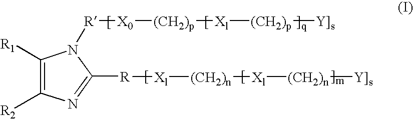
formulas IIa and IIb (compound class B) 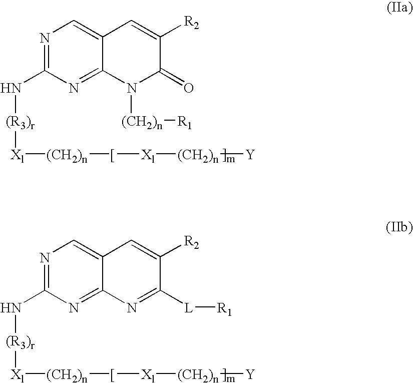
wherein
each L is independently selected from —NH—CO—NH—, —NH—SO₂—, or —NH—CS—NH—,
each X is independently selected from —CH₂—, —NH—, —O—, —S—, 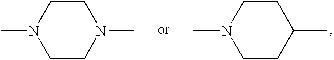
each Y is independently selected from —NH₂, —NHR₁, —OH, —SH or —SO(CH₃),
each l is independently selected to be 0 or 1,
each m is independently selected to be an integer from 0 to 10,
each n is independently selected to be an integer from 0 to 10,
each o is independently selected to be 0 or 1,
each p is independently selected to be an integer from 0 to 10,
each q is independently selected to be an integer from 0 to 10,
each r is independently selected to be an integer from 0 to 2,
R and R′ are independently of each other —H, 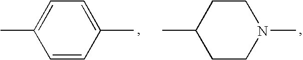
and each s is independently selected to be 0 or 1, with the provisio that s=0 if R or R′ is H,
each R₁ is independently selected from —H, C₁-C₆ alkyl (linear or branched), bicyclo[3.3.1]heptanyl,or an unsubstituted or partially or fully substituted C₃-C₈ cycloalkyl, aryl, pyridinyl or pyrimidinyl, substituted by —F, —Cl, —Br, —I, —CN, —OH, —SH, —NH₂, —NHCHR₂R₂, C₁-C₆-alkoxy, C₁-C₆-alkylthio, C₁-C₆-haloalkyloxy, partially or fully halogenated C₁-C₆ alkyl and/or —X₁—(CH₂)ₙ—Y (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
each R₂ is independently selected from —H, C₁-C₆ alkyl (linear or branched), an unsubstituted or partially or fully substituted aryl, substituted by —F, —Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), and
each R₃ is independently selected from X, an unsubstituted or partially or fully substituted aryl, pyridinyl or pyrimidinyl, substituted by —F, —Cl, —Br, —I, —CN, —OH, —SH, —NH₂, —NHCHR₁R₁, C₁-C₆-alkoxy, C₁-C₆-alkylthio, C₁-C₆-haloalkyloxy, and/or partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
formulas IIIa and IIIb (compound class C) 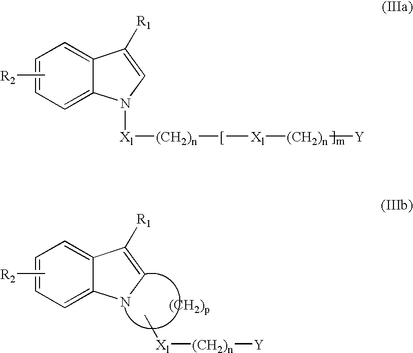
wherein
each R₁ is independently selected from 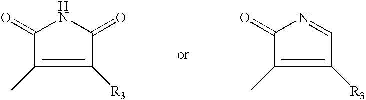
each R₃ is independently selected from -indolyl, N—(C₁-C₆ alkyl) -indolyl (alkyl is linear or branched), —NHR₁′, —S—R₁′, or —O—R₁′,
R₁′ is —H, C₁-C₆ alkyl (linear or branched) or aryl,
each R₂ is independently selected from —H, —F, —Cl, —Br, —I, —CN, —OH, —SH, —NH₂, C₁-C₆-alkyl (linear or branched), C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
each X is independently selected from —CH₂—, —NH—, —O—, —S—, 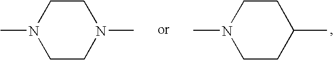
each Y is independently selected from —NH₂, —NHR₁, —OH, —SH or —SO(CH₃),
each l is independently selected to be 0 or 1,
m is an integer from 0 to 10,
each n is independently selected to be an integer from 0 to 10,
p is an integer from 2 to 6
formula IV (compound class D) 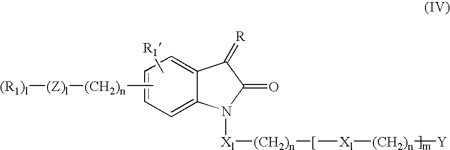
wherein
each X is independently selected from —CH₂—, —NH—, —O—, —S—, 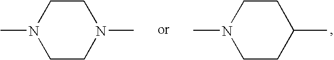
each Y is independently selected from —NH₂, —NHR₁, —OH, —SH or —SO(CH₃), Z is —SO₂—NR₁R₁, —CO—, —O—CO—, —NH—CO—, —COO—, —CO—NH—, —OCH₂—, —SCH₂—,
each l is independently selected to be 0 or 1,
m is an integer from 0 to 10,
each n is independently selected to be an integer from 0 to 10,
R is ═CR₁L, ═N—NH-L
each R₁ is independently selected from —H, C₁-C₆ alkyl (linear or branched), unsubstituted or partially or fully substituted aryl, pyridinyl, pyrimidinyl, C₃-C₈ cycloalkyl substituted by —F, Cl, —Br, —I, —CN, —OH, —SH, —NH₂, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group, C₁-C₆-alkylthio denotes an S-alkyl group, C₁-C₆-haloalkyloxy denotes an halogen-alkyl-O group, C₁-C₆-haloalkyl denotes an halogen-alkyl group wherein the alkyl group is linear or branched), —F, —Cl, —Br, —I, —COOH, —(CH₂)ₙ—OH, oxazolyl, thiazolyl, thienyl, pyrrolyl, furanyl, imidazolyl, pyrazolyl, pyridinyl, pyrimidinyl,
R₁′ is independently selected from H or R₁ and R₁′ may formn together the following substituted ringsystem 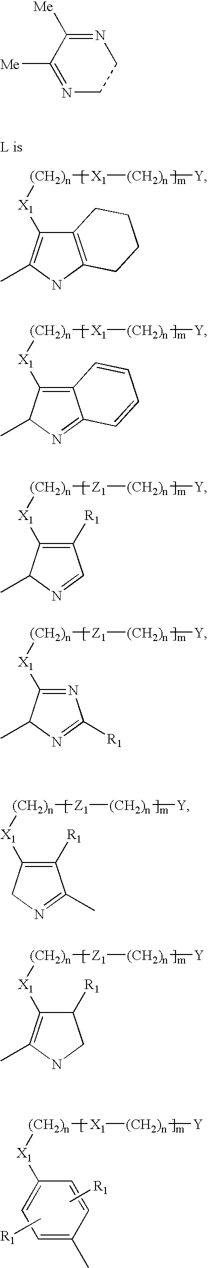
formulas V and VI (compound class E and F) 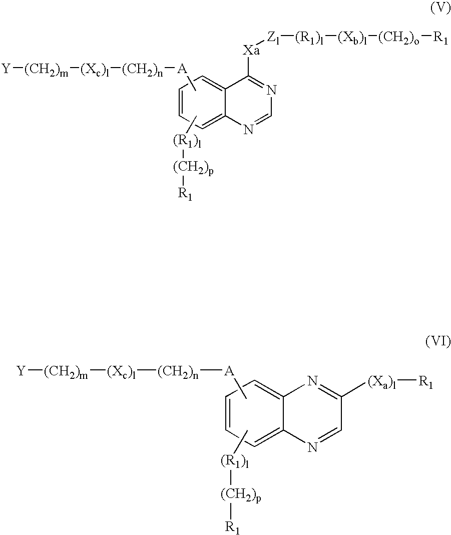
wherein
A, Xₐ, X_{b}, and X_{c}, are independently selected to be Z, —CH₂—, —NH—, —O—, —S—, 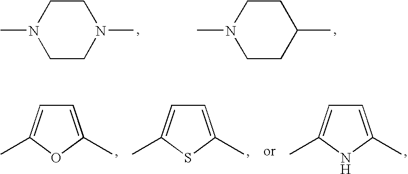
each Y is independently —NH₂, —NHR₁, —OH, —SH or —SO(CH₃),
each Z is independently selected from —SO₂—NR₁—, —CO—, —O—CO—, —NH—CO—, —COO—, —CO—NH—, —CS—NH—, —OCH₂—, —SCH₂—, or —NH—CO—NH—,
each l is independently selected to be 0 or 1,
each m is independently selected to be an integer from 0 to 10,
each n is independently selected to be an integer from 0 to 10,
each o is independently selected to be an integer from 0 to 10,
each p is independently selected to be an integer from 0 to 10,
each R₁ is independently selected from —H, —O—, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or partially or fully substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), 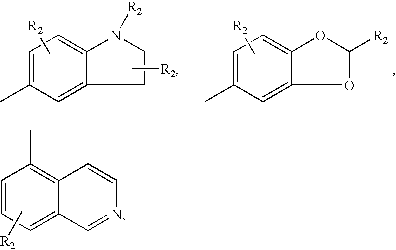
each R₂ is independently selected from —F, —Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
formula VII (compound class G) 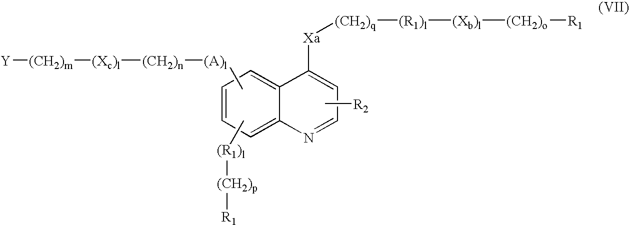
formula VIII (compound class H) 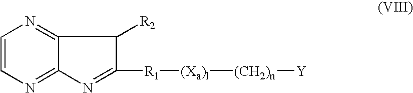
formula IX (compound class I) 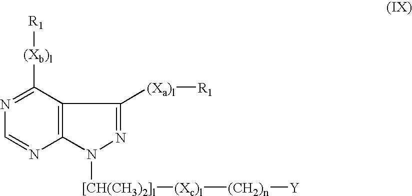
formula X (compound class J) 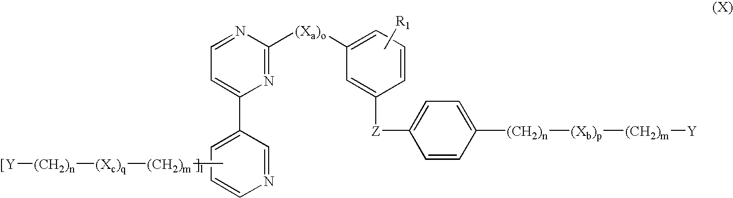
or formula XI (compound class K) 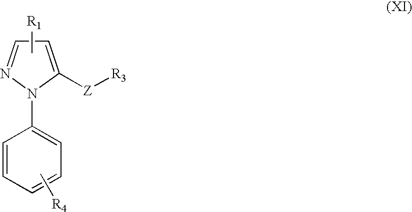
wherein
A, Xₐ, X_{b}, and X_{c} are independently selected from Z, —CH₂—, —NH—, —O—, —S—, 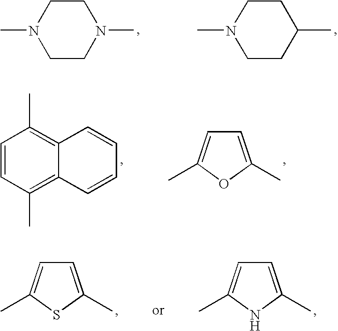
each Y is independently selected from —H, —NH₂, —NHR₁, —OH, —SH or —SO(CH₃),
each Z is independently selected from —SO₂—NR₁—, —CO—, —O—CO—, —NH—CO—, —COO—, —CO—NH—, —OCH₂—, —SCH₂—, —NH—CO—NH—,
each l is independently selected to be 0 or 1,
each m is independently selected to be an integer from 0 to 10,
each n is an integer independently selected from 0 to 10,
each of o, p, q is an integer independently selected from 0 to 10,
each R₁ is independently selected from —H, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁- C₆ alkyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), —F, —Cl, —Br, —I, —COOH, —NH₂, 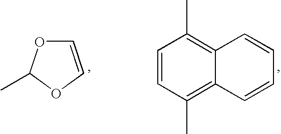
each R₂ is independently selected from —F, —Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, partially or fully halogenated C₁-C₆ alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched),
R₃ is —H or —(R₁)₁—(Xₐ)₁—(CH₂)ₙ—(X_{b})₁—(CH₂)ₙ—(Y)₁—R₁,
R₄ is —H or —(R₁)₁—(Z)₁—(CH₂)ₙ—(X_{b})₁—(CH₂)ₙ—(Y)₁—R₁
covalently on a support material via the group Y;
b) bringing the pool of proteins containing at least one protein kinase into contact with at least one of the compounds immobilized on the support material; and
c) separating the proteins not bound to the at least one compound immobilized on the support material from the at least one protein kinase bound to the at least one compound immobilized on the support material.

60. The method according to claim 59, wherein the at least one compound immobilized on the support material is 4-[4-(4-fluoro-phenyl)-5-pyridine-4-yl-1H-imidazole-2-yl]-benzylamine, 2-[4-(2-Amino-ethoxy)-phenylamino]-6-(2,6-dichloro-phenyl)-8-methyl-8H-pyrido[2,3-d]pyrimidine-7-one, 2-[1-(3-aminopropyl)-1H-indole-3-yl)maleimide, 3-[1-(3-Aminopropyl)-1H-indol-3-yl]-3-(1H-indol-3-yl)-maleimide, 3-[1-(3-Aminopropyl)-1H-indol-3-yl]-4-(1-methyl-1H-indol-3-yl)maleimide, 3-(8-Aminomethyl-6,7,8,9-tetrahydropyrido-[1,2-a]-indol-10-yl)-4-(1-methyl-1H-indol-3-yl)-maleimide, [6-(3-Amino-propoxy)-7-methoxy-quinazolin-4-yl]-(3-chloro-phenyl)-amine, 6-(3-Amino-propoxy)-7-methoxy-quinazolin-4-yl]-(3-chloro-4-fluoro-phenyl)-amine, 6-(3-Amino-propoxy)-7-methoxy-quinazolin-4-yl]-(3-bromo-phenyl)-amine and 4-[4-(4-Amino-butyl)-piperazin-1-yl-methyl]-N-[4-methyl-3-(4-pyridin-3-yl-pyrimidin-2-ylamino)-phenyl]-benzamide.

61. The method according to claim 59, further comprising a step:
d) releasing the at least one protein kinase bound to the at least one compound immobilized on the support material from the at least one of said compounds and collecting the released at least one protein kinase.

62. The method according to claim 59, wherein the support material comprises agarose and/or modified agarose material.

63. The method according to claim 59, wherein the support material comprises ferro- or ferrimagnetic particles.

64. The method according to claim 59, wherein in step c) the separation of the proteins not bound to the at least one compound immobilized on the support material from the at least one ATP binding protein bound to the at least one compound immobilized on the support material is effected by washing with a buffer containing 5 to 500 mM Hepes pH 6.5-8.5 or 5 to 500 mM Tris-HCl pH 6.8 to 9.0, 0 to 1000 mM NaCl, 0.0 to 5% Triton X-100, 0 to 500 mM EDTA, and 0 to 200 mM EGTA.

65. The method according to claim 64, wherein the buffer contains 20 mM Hepes/NaOH pH 7.5, 150 mM NaCl, 0.25% Triton X-100, 1 mM EDTA, and 1 mM EGTA.

66. The method according to claim 61, wherein in step d) the release of the at least one protein kinase bound to the at least one compound immobilized on the support material is effected by washing with a buffer containing 5 to 500 mM Hepes pH 6.5-8.5 or 5 to 500 mM Tris-HCl pH 6.8 to 9.0, 0 to 1000 mM NaCl, 0.0 to 5.0% Triton X-100, 0 to 500 mM EDTA, 0 to 200 mM EGTA, 1 to 100 mM ATP, 1 to 200 mM MgCl₂ and 0.1 to 10 mM of at least one of the compounds immobilized on the support material.

67. The method according to claim 66., wherein the buffer contains 20 mM Hepes pH 7.5, 150 mM NaCl, 0.25% Triton X-100, 1 mM EDTA, 1 mM EGTA, 10 mM ATP, 20 mM MgCl₂ and 1 mM of at least one of the compounds immobilized on the support material.

68. The method according to claim 59, wherein the pool of proteins is a proteome, cell lysate or tissue lysate.

69. The method according to claim 59, wherein the ATP binding protein is a protein kinase.

70. The method according to claim 59, wherein the at least one ATP binding protein is enriched at least 100-fold from the pool of proteins.

71. The method according to claim 70, wherein the at least one ATP binding protein is enriched at least 10⁴-fold and preferably up to 10⁶-fold.

72. Kit comprising a medium according to general formula (I) or general formula (IIa) or general formula (IIb).

73. Kit according to claim 72, further comprising at least one buffer containing 5 to 500 mM Hepes pH 6.5-8.5 or 5 to 500 mM Tris-HCl pH 6.8 to 9.0, 0 to 1000 mM NaCl, 0.0 to 5% Triton X-100, 0 to 500 mM EDTA, and 0 to 200 mM EGTA.

74. Kit according to claim 72, further comprising at least one buffer containing 20 mM Hepes/NaOH pH 7.5, 150 mM NaCl, 0.25% Triton X-100, 1 mM EDTA, and 1 mM EGTA.

75. Kit according to claim 72, further comprising at least one buffer containing 5 to 500 mM Hepes pH 6.5-8.5 or 5 to 500 mM Tris-HCl pH 6.8 to 9.0, 0 to 1000 mM NaCl, 0.0 to 5.0% Triton X-100, 0 to 500 mM EDTA, 0 to 200 mM EGTA, 1 to 100 mM ATP, 1 to 200 mM MgCl₂ and 0.1 to 10 mM.

76. Method of making a quinazoline compound of general formula (O) or a salt thereof: 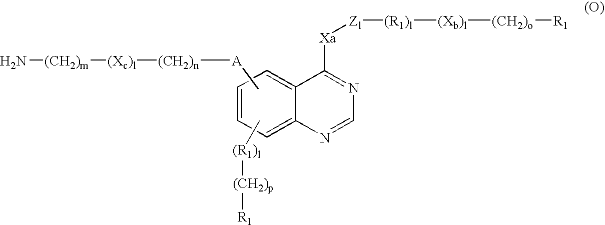
the method comprising the step (A):
reacting a compound with the general formula (Φ) 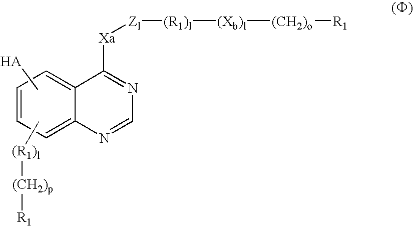
with a compound of the general formula (θ) 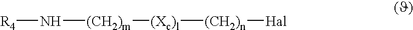
to give compound (K) 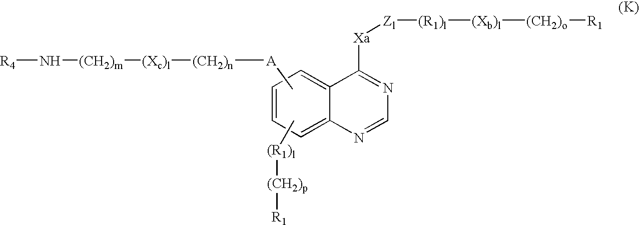
wherein the reaction is carried out in the presence of a base and an inert solvent,
and wherein
A is —O—, —S—, —NH—,
Hal is —Cl, —Br, or —I,
Xₐ, X_{b}, and X_{c} are independently selected from Z, —CH₂—, —NH—, —O—, —S—, 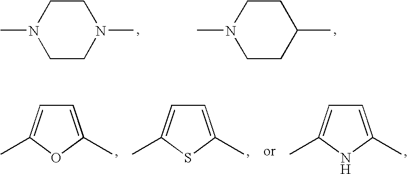
Z is —SO₂—NR₁—, —CO—, —O—CO—, —NH—CO—, —COO—, —CO—NH—, —CS—NH—, —OCH₂—, —SCH₂—, or —NH—CO—NH—,
l is independently selected for each moiety to be 0 or 1,
each of m, n, o, and p is an integer independently selected for each moiety from 0-10,
R₁ is independently selected from —H, —O—, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or partially or fully substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—(CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), 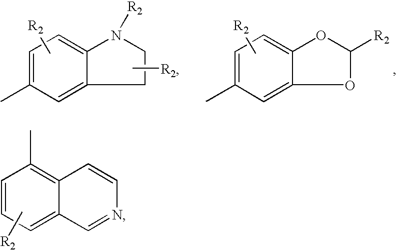
R₂ is —F, —Cl, —Br, —I, —CN, —OH, —SH, —NH₂, C₁-C₆ alkyl (linear or branched), C₁-C₆-alkoxy, C₁-C₆-alkylthio, C₁-C₆-haloalkyloxy, partially or fully halogenated, C₁-C₆ alkyl (C₁-C₆-alkoxy denotes an 0-alkyl group, C₁-C₆-alkylthio denotes an S-alkyl group, C₁-C₆-haloalkyloxy denotes an halogen-alkyl-O group, C₁-C₆-haloalkyl denotes an halogen-alkyl group wherein the alkyl group is linear or branched),
R₄ is a leaving group, selected from the group consisting of t-butyloxycarbonyl (BOC), flourene-9-ylmethoxycarbonyl (Fmoc) or benzyloxycarbonyl and further comprising as step (B):
cleaving off the leaving group R₄ to give compound (O) or a salt thereof.

77. The method according to claim 76, wherein the base used in reaction step (A) is K₂CO₃ or Na₂CO₃ and the inert solvent is selected from the group consisting of acetonitrile, acetone, toluene, THF or DMF.

78. The method according to claim 76, wherein in compounds (Φ), (θ) and (K),
l is 0, o is 0, p is 0, and m is 0,
n is an integer selected from 1 to 8, preferably from 2 to 6, and most preferably is 4,
X is —NH—,
R₃ is C₁-C₆ alkyl (linear or branched), and
R₁ is an unsubstituted or partially or fully substituted aryl, wherein the aryl is substituted by at least one of the substituents comprised in the group consisting of —F, —Cl, —Br, —I, —CN, —OH, —SH—, C₁-C₆-alkylthio, and benzyloxy-.

79. The method according to claim 76, wherein compound (Φ) is selected from the group consisting of 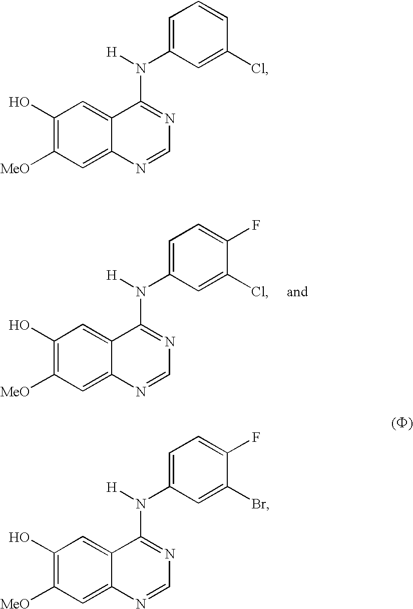
and compound (θ) is

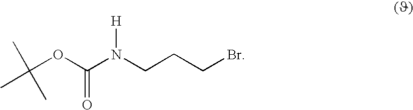

80. The method according to claim 76, wherein the leaving group R₄ is removed from compound (K) by contacting compound (K) with hydrochloric acid as the protonic acid, preferably by contacting compound (K) with a solution of hydrochloric saturated ethylacetate.

81. A method of making a compound with the general formula (Σ) 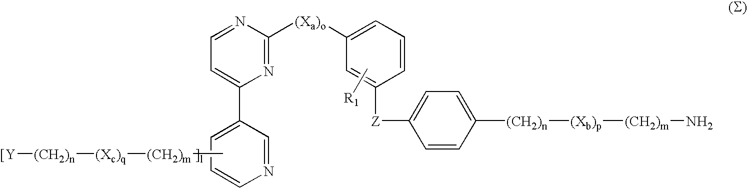
comprising the steps (A):
reacting a compound of the general formula (T) 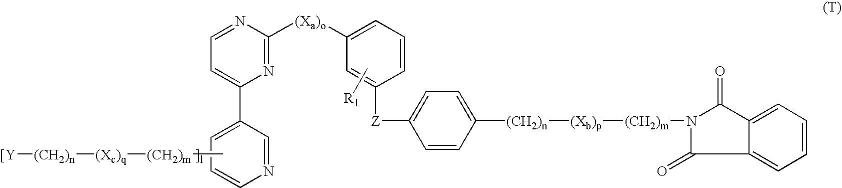
with hydrazine in a protic solvent and subsequently reacting the crude reaction product with an aqueous solution of an protonic acid, wherein in compounds (Σ) and (T) Xₐ, X_{b} and X_{c} are independently selected from the group consisting of Z, —CH₂—, —NH—, —O—, —S—, 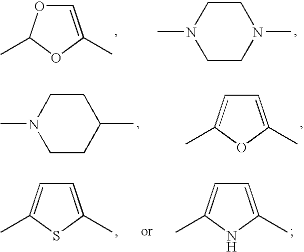
Y is —NH₂, —NHR₁, —OH, —SH or —SO(CH₃),
Z is —SO₂—NR₁, —CO—, —O—CO—, —NH—CO—, —COO—, —CO—NH—, —OCH₂—, or —SCH₂—,
l is independently selected to be 0 or 1,
m is an integer independently selected from 0 to 10,
n is an integer independently selected from 0 to 10,
o is an integer independently selected from 0 to 10,
p is an integer independently selected from 0 to 10,
q is an integer independently selected from 0 to 10, and
R₁ is independently selected from —H, C₁-C₆ alkyl (linear or branched), C₁-C₆-alkoxy, C₁-C₆-alkylthio, C₁-C₆-haloalkyloxy, C₁-C₆ partially or fully halogenated alkyl, unsubstituted or substituted C₃-C₈ cycloalkyl, an unsubstituted or partially or fully substituted aryl, wherein the cycloalkyl and the aryl are optionally substituted by —F, —Cl, —Br, —I, —CN, —OH, —SH, —NH₂, —CONH₂, C₁-C₆ alkyl (linear or branched), —C≡C—CH₂)ₙ—CH₃, C₁-C₆-alkoxy, C₁-C₆-alkylthio, C₁-C₆-haloalkyloxy, and/or C₁-C₆ partially or fully halogenated alkyl (C₁-C₆-alkoxy denotes an O-alkyl group wherein the alkyl group is linear or branched, C₁-C₆-alkylthio denotes an S-alkyl group wherein the alkyl group is linear or branched, C₁-C₆-haloalkyloxy denotes an halogen-alkyl-O group wherein the alkyl group is linear or branched, C₁-C₆-haloalkyl denotes an halogen-alkyl group wherein the alkyl group is linear or branched), —F, —Cl, —Br, —I, —COOH, —NH₂,

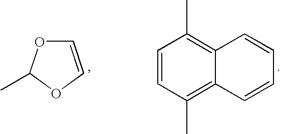

82. The method of claim 81, wherein the protic solvent is selected from the group of alkyl alcohols, preferably from the group consisting of methanol, ethanol, propanol, iso-propanol, n-butanol and and iso-butanol, and most preferably is ethanol.

83. The method according to claim 81, wherein the protonic acid is selected from hydrochloric acid or hydrobromic acid, and preferably is hydrochloric acid.

84. The method according to claim 81, wherein (Σ) is 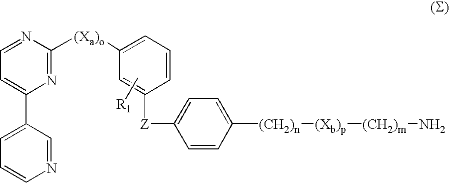
and wherein o is 1 and Xₐ is —NH—,
Z is —CO—NH—,
R₁ is an alkyl group,
and X_{b} is:

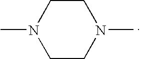

85. The method according to claim 81, wherein
l is 0, m is 0, and
n is an integer selected from 1 to 6, preferably from 2 to 4 and most preferably is 4.

86. The method according to claim 81, wherein the compound (Σ) is

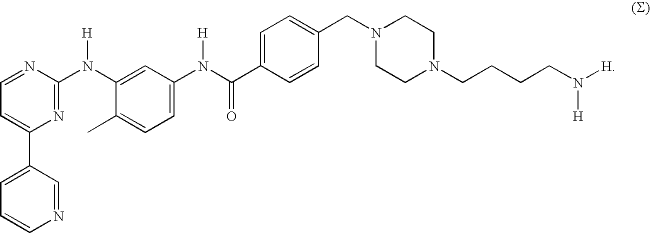

87. The method according to claim 81, further comprising the step of providing the compound (T) by reaction of compound (Λ) or a salt thereof 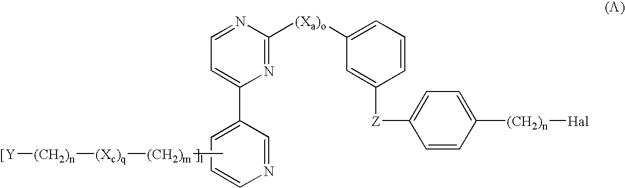
with compound (M) or salt thereof 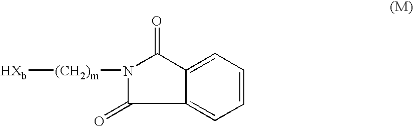
in the presence of a base,
wherein Hal is a halogen selected, preferably selected from the group consisting of —Cl, —Br, and —I, and preferably is —Br, and
wherein Xₐ, X_{b} and X_{c}, Y, Z, R₁, l, m, n, o, p, and q have the same meaning as in compounds (T) and (Σ) as defined in claim 79.

88. The method according to claim 87, wherein the base is selected from the group consisting of ammonia, primary amines, especially primary alkyl amines, secondary amines, especially secondary alkylamines or tertiary amines, especially tertiary alkylamines, and preferably is triethylamine.

89. The method according to claim 87, wherein in compound (Λ)
l is 0
Xₐ is —NH—,
R₁ is a linear or branched C₁-C₆-alkyl, preferably —CH₃,
Z is —NHCO—,
n is an integer from 1 to 8, preferably from 1 to 4, and most preferably is 1, and
X_{b} is [chemical structure]
